(19) United States
(12) Patent Application Publication
(10) Pub. No.: US 2023/0212318 A1
(43) Pub. Date: Jul. 6, 2023

(54) PROTEASE-ACTIVATABLE BISPECIFIC PROTEINS

(71) Applicant: AMGEN INC., Thousand Oaks, CA (US)

(72) Inventors: Wei Yan, Thousand Oaks, CA (US); Martin J. Pentony, Seattle, WA (US); Mark L. Michaels, Encino, CA (US); Patrick Baeuerle, Gauting (DE)

(21) Appl. No.: 17/727,316

(22) Filed: Apr. 22, 2022

Related U.S. Application Data
(63) Continuation of application No. 15/513,011, filed on Mar. 21, 2017, now abandoned.
(60) Provisional application No. 62/055,330, filed on Sep. 25, 2014.

(51) Int. Cl. C07K 16/46; C07K 14/725; C07K 16/28; C07K 16/32

(52) U.S. Cl. CPC C07K 16/468; C07K 14/7051; C07K 16/32; C07K 16/2809; C07K 2317/31; C07K 2317/52; C07K 2317/64; C07K 2317/73; C07K 2317/92; C07K 2317/565; C07K 2317/622; C07K 2319/00; C07K 2319/50

(57) ABSTRACT

Described herein are protease-activatable proteins (PABPs), which, when activated, can mediate cytolysis of target cells by effector cells. Also provided are nucleic acids encoding such PABPs and methods of making and using PABPs.

### FIELD

The invention is in the field of protein engineering.

### BACKGROUND

Bispecific antibodies have shown promise as cancer therapeutics. For example, a bispecific antibody that targets both CD3 and CD19 in a Bispecific T cell Engager (BiTE®) format has shown impressive efficacy at low doses. Bargou et al. (2008), Science 321: 974-978. The BiTE® format consists essentially of two scFv’s, one of which targets CD3 and one of which targets a tumor antigen, joined by a linker. The resulting antibody has a short half life in vivo and therefore requires dosing by continuous infusion. Bispecific formats with improved pharmacokinetic properties may be desirable to eliminate the need for continuous dosing. However, formats with longer half lives could imaginably cause prolonged and poorly localized T cell activation, leading to undesirable side effects, since engagement of CD3 can cause T cell activation. Tsoukas et al. (1985), J. Immunol. 135(3): 1719-1723. Hence, there is a need in the art for bispecific antibody formats that have reasonably long half lives, but are activated specifically in a disease microenvironment, for example, in the vicinity of a tumor.

### SUMMARY

Broadly speaking, herein are described protease-activatable bispecific proteins (PABPs), nucleic acids encoding PABPs, methods of making PABPs, and methods of using PABPs. Such PABPs comprise at least a portion that binds to a target cell, a portion that binds to an effector cell, and a protease cleavage site.

In more detail, described herein is a protein comprising: (a) one or more polypeptide chain(s) that bind to a target cell; (b) one or more polypeptide chain(s) that bind to an effector cell; (c) a third polypeptide; and (d) a linker comprising a protease cleavage site that links the third polypeptide of (c) to the remainder of the protein; wherein either the protein binds to a target cell more effectively or the protein binds to an effector cell more effectively when the protease cleavage site is essentially completely cleaved as compared to binding observed when the protease cleavage site is uncleaved and/or wherein the Ec50 of the protein in a cell cytolysis assay when the protease cleavage site is essentially completely cleaved is not more than a fifth of the Ec50 of the protein in the same assay when the protease cleavage site has not been cleaved. The polypeptide chain(s) of (a) can comprise a first pair of immunoglobulin heavy and light chain variable regions (VH1 and VL1) that bind to the target cell when part of an IgG or scFv antibody and the polypeptide chain(s) of (b) can comprise a second pair of immunoglobulin heavy and light chain variable regions (VH2 and VL2) that bind to the effector cell when part of an IgG or scFv antibody. The effector cell can be a T cell or an NK cell. The VH2 and VL2 can bind to a polypeptide that is part of a TCR-CD3 complex when part of an IgG or scFv antibody, for example, human CD3ε. VH2 can comprise a heavy chain CDR1, CDR2, and CDR3 comprising the amino acid sequences of SEQ ID NOs: 42, 43, and 44, respectively, and VL2 can comprise a light chain CDR1, CDR2, and CDR3 comprising the amino acid sequences of SEQ ID NOs: 47, 48, and 49, respectively. VH2 and VL2 can comprise the amino acid sequences of SEQ ID NOs: 40 and 45, respectively. In some embodiments, the protease cleavable site can be cleaved by MMP-2, MMP-9, or MMP-11. In some embodiments, the protease cleavable site can comprise an amino acid sequence selected from the group consisting of: GPLGIAGQ (SEQ ID NO:1), GGPLGMLSQS (SEQ ID NO:2), PLGLAG (SEQ ID NO:3), RRRRR (SEQ ID NO:4), RRRRRR (SEQ ID NO:82), GQSSRHRRAL (SEQ ID NO:5), AANLRN (SEQ ID NO:95), AQAYVK (SEQ ID NO:96), AANYMR (SEQ ID NO:97), AAALTR (SEQ ID NO:98), AQNLMR (SEQ ID NO:99), and AANYTK (SEQ ID NO:100).

In one aspect, the protein can comprise a first polypeptide chain comprising an amino acid sequence having the formula: VH1-L1-VL1-L2-VH2-L3-VL2-X1, wherein L1, L2 and L3 are linkers, L3 can be present or absent, and X1 is a half life-extending moiety, for example an Fc polypeptide chain, and a second polypeptide chain comprising an amino acid sequence having the formula: Y-L4-X2, wherein Y is the polypeptide of (c) descrbided above, L4 is the linker comprising the protease cleavage site of (d) described above, and X2 is a half life-extending moiety, for example, an Fc polypeptide chain. The first polypeptide chain can comprise the amino acid sequence of SEQ ID NO:30, and the second polypeptide chain can comprise the amino acid sequence of SEQ ID NO:36 or SEQ ID NO:38.

In another aspect, the protein can comprise a first polypeptide chain comprising an amino acid sequence having the formula VH1-L4-VL2-L5-CL-X1, wherein L4 and L5 are a linkers and can be present or absent, CL is a light chain constant region, and X1 is a half life-extending moiety and can be present or absent, and a second polypeptide chain having the formula Y-L1-VH2-L2-VL1-L3-CH1-X2, wherein Y is the polypeptide of (c) described above, L1 is the linker comprising the protease cleavage site of (d) described above, L2 and L3 are linkers and can be present nor absent, CH1 is a first heavy chain constant region, and X2 is a half life-extending moiety and can be present or absent. X1 and X2 can be an Fc polypeptide chains, and both can be present. The first polypeptide chain an comprise the amino acid sequence of SEQ ID NO:6, and the second polypeptide chain can comprise the amino acid sequence of SEQ ID NO:10, 12, 14, 16, or 18.

In a further aspect, the protein can comprise a first polypeptide chain comprising an amino acid sequence having the formula VH1-L4-VL1-L5-X1 or VL1-L4-VH1-L5-X1, wherein L4 and L5 are linkers and can be present or absent, and X1 is an Fc polypeptide chain, and a second polypeptide comprising an amino acid sequence having the formula Y-L1-VH2-L2-VL2-L3-X2 or Y-L1-VL2-L2-VH2-L3-X2 wherein Y is the polypeptide of (c) described above, L1 is the linker comprising the protease cleavage site of (d) described above, L2 and L3 are linkers and can be present or absent, and X2 is an Fc polypeptide chain. The first polypeptide chain can comprise the amino acid sequence of SEQ ID NO:20, and the second polypeptide chain can comprise the amino acid sequence of SEQ ID NO: 24, 26, or 28.

The target cell of any of the PABPs described herein can be a cancer cell. In this case, VH1 and VL1 may, when part of an scFv or IgG antibody, bind to a protein selected from the group consisting of: epidermal growth factor receptor (EGFR), EGFRvlll, melanoma-associated chondroitin sulfate proteoglycan (MCSP), mesothelin (MSLN), folate receptor 1 (FOLR1), CD33, CDH19, or epidermal growth factor 2 (HER2).

In some embodiments, a protein as described herein can comprise one of the following pairs of polypeptide chains: (a) a first polypeptide chain comprising an amino acid sequence having the following formula: VH1-CH1 -L1-VH2-CH1, wherein VH1 and VH2 are immunoglobulin heavy chain variable regions, CH1 is a first heavy chain constant region, and L1 is a linker comprising a protease cleavable site, and a second polypeptide chain comprising an amino acid sequence having the following formula: VL1-CL-L2-VL2-CL, wherein VL1 and VL2 are immunoglobulin light chain variable regions, CL is a light chain constant region, and L2 is a linker that does not contain a protease cleavage site; (b) a first polypeptide chain comprising an amino acid sequence having the following formula: VH1-CH1-L1-VL2-CL, wherein VH1 is an immunoglobulin heavy chain variable region, VL2 is an immunoglobulin light chain variable region, CH1 is a first heavy chain constant region, CL is a light chain constant region, and L1 is a linker comprising a protease cleavage site, and a second polypeptide chain comprising an amino acid sequence having the following formula: VL1-CL-L2-VH2-CH1, wherein VL1 is an immunoglobulin light chain variable regions, VH2 is an immunoglobulin heavy chain variable region, L2 is a linker that does not contain a protease cleavage site, and CH1 is a first heavy chain constant region; (c) a first polypeptide chain comprising an amino acid sequence having the following formula: VL1-CL-L1-VL2-CL, wherein VL1 and V2 are immunoglobulin light chain variable regions, CL is a light chain constant region, and L1 is a linker comprising a protease cleavage site, and a second polypeptide chain comprising an amino acid sequence having the following formula: VH1-CH1-L2-VH2-CH1, wherein VH1 and VH2 are heavy chain variable regions, L2 is a linker that does not contain a protease cleavage site, and CH1 is a first heavy chain constant region; (d) a first polypeptide chain comprising an amino acid sequence having the following formula: VL1-CL-L1-VH2-CH1, wherein VH2 is an immunoglobulin heavy chain variable region,VL1 is an immunoglobulin light chain variable region, CH1 is a first heavy chain constant region, CL is a light chain constant region, and L1 is a protease-cleavable linker, and a second polypeptide chain comprising an amino acid sequence having the following formula: VH1-CH1-L2-VL2-CL, wherein VL2 is an immunoglobulin light chain variable regions, VH1 is an immunoglobulin heavy chain variable region, L2 is a linker that does not contain a protease cleavage site, CH1 is a first heavy chain constant region, and CL is a light chain constant region; wherein VL1 and VH1 bind to a target cell when part of an IgG or scFv antibody and VL2 and VH2 bind to an effector cell when part of an IgG or scFv antibody. The effector cell can be a T cell. The VH2 and VL2 can bind to a protein that is part of a TCR-CD3 complex when part of an IgG or scFv antibody, for example, human CD3ε. The VH2 and VL2 can comprise an immunoglobulin heavy chain CDR1, CDR2, and CDR3 comprising the amino acid sequence of SEQ ID NOs: 42, 43, and 44, respectively, and an immunoglobulin light chain CDR1, CDR2, and CDR3 comprising the amino acid sequence of SEQ ID NOs: 47, 48, and 49, respectively. The VH2 and VL2 can comprise the amino acid sequences of SEQ ID NOs: 40 and 45, respectively. The protease cleavage site can comprise an amino acid sequence selected from the group consisting of GPLGIAGQ (SEQ ID NO:1), GGPLGMLSQS (SEQ ID NO:2), PLGLAG (SEQ ID NO:3), AANLRN (SEQ ID NO:95), AQAYVK (SEQ ID NO:96), AANYMR (SEQ ID NO:97), AAALTR (SEQ ID NO:98), AQNLMR (SEQ ID NO:99), and AANYTK (SEQ ID NO:100). The target cell can be a cancer cell. The VH1 and VL1 may bind to epidermal growth factor receptor (EGFR), EGFRvlll, melanoma-associated chondroitin sulfate proteoglycan (MCSP), mesothelin (MSLN), folate receptor 1 (FOLR1), CD33, CDH19, or epidermal growth factor 2 (HER2) when part of an IgG or scFv antibody.

In another aspect, described herein is a nucleic acid encoding any of the PABPs described above or below. Also provide are vectors and host cells containing such nucleic acids. Exemplary pairs of nucleic acids encoding PABPs include, without limitation, nucleic acid comprising the following sequences: SEQ ID NOs:7 and 11; SEQ ID NOs:7 and 13; SEQ ID NOs:7 and 15; SEQ ID NOs:7 and 17; SEQ ID NOs:7 and 19; SEQ ID NOs:21 and 25; SEQ ID NOs:21 and 27; SEQ ID NOs:21 and 29; SEQ ID NOs:31 and 37; and SEQ ID NOs:31 and 39. Also described herein is a method of making any of the PABPs described herein comprising culturing a host cell containing a nucleic acid encoding the PABP under conditions such that the PABP is expressed, and recovering the PABP from the culture medium or the cell mass.

In a further aspect, described herein is a method for treating a cancer patient comprising administering a therapeutically effective dose of a PABP as described herein. This method includes, in some embodiments, administration of radiation, a chemotherapeutic agent, and/or a non-chemotherapeutic anti-neoplastic agent before, after, and/or concurrently with administration of a PABP. The cancer cells of the patient can express a protease that can cleave a protease cleavage site that is part of the PABP.

In another aspect, described herein is a method for treating a patient suffering from an infection, a fibrotic disease, a neurodegenerative disease, or an autoimmune or inflammatory disease comprising administering a therapeutically effective dose of a PABP as described herein.

### BRIEF DESCRIPTION OF THE FIGURES

FIG. 1: Exemplary diagram of a protease-activatable bispecific protein (PABP). The numbered items signify as follows: oval labeled “1” represents Component 1, which binds to a target molecule, as defined herein; oval labeled “2” represents Component 2, which binds to an effector cell molecule, as defined herein; oval labeled “3” represents Component 3, an optional moiety, optionally a polypeptide, that binds to Component 1 or 2 and blocks its binding to a target cell or an effector cell, respectively; dotted line labeled “4” represents Component 4, an amino acid sequence cleavable by a protease, which may include further linker sequences; rectangle labeled “5” represents Component 5, an optional, half-life extending moiety, which can, optionally, be a polypeptide. The solid, curving line extending from the oval labeled “3” is a non-cleavable linker that, for example, can be a polypeptide.

FIG. 2: Diagram of an embodiment of a PABP. The ovals labeled VH1 and VL1 stand for immunoglobulin heavy and light chain variable (VH and VL) regions, respectively, which comprise Component 1, as indicated, and bind to a target cell when they are part of an IgG or scFv antibody. The ovals labeled VH2 and VL2 represent VH and VL regions, respectively, that bind to CD3ε when they are part of an IgG or scFv antibody and that comprise Component 2, as indicated. The smaller oval labeled “CD3ε” is all or a part of CD3ε, which represents Component 3, as indicated. The ovals labeled CH2 and CH3 represent the second and third constant domains, respectively, of an IgG antibody. Together with part of all of the hinge region, these two domains form an Fc polypeptide chain. The two Fc polypeptide chains represent Component 5, as indicated. The dotted line labeled “4” represents Component 4, as indicated, which comprises a protease cleavage site. Solid lines represent peptide linkers (curving lines) or hinge regions (straight lines).

FIG. 3: Diagram of an embodiment of a PABP. All labeled ovals and solid and dashed lines have the same meanings as in FIG. 2. The rectangles labeled “CH1” and “CL” represent immunoglobulin CH1 and CL regions.

FIG. 4: Diagram of an embodiment of a PABP. All labeled ovals and solid and dashed lines have the same meanings as in FIG. 2.

FIG. 5A: Diagram of an embodiment of a PABP. All labeled ovals and solid and dashed lines have the same meanings as in FIGS. 2 and 3.

FIG. 5B: Diagram of an embodiment of a PABP. All labeled ovals and solid and dashed lines have the same meanings as in FIGS. 2 and 3.

FIG. 6: Digestion of PABP and control molecules with MMP-2. Methods are described in Example 2, and the digestion products were run on an SOS-PAGE gel under reducing conditions. Lanes contain the following samples: 1) CD3ε(1-27)-aCD3-aHER2-Xbody without MMP-2; 2) CD3ε(1-27)-aCD3-aHER2-Xbody with MMP-2; 3) CD3ε(1-27)-MMP-2csV1-aCD3-aHER2-Xbody without MMP-2; 4) CD3ε(1-27)-MMP-2csV1-aCD3-aHER2-Xbody with MMP-2; 5) CD3ε(1-27)-FURINcsV1-aCD3-aHER2-Xbody without MMP-2; 6) CD3ε(1-27)-FURINcsV1-aCD3-aHER2-Xbody with MMP-2; 7) CD3ε(1-27)-MMP-2csV2-aCD3-aHER2-Xbody without MMP-2; 8) CD3ε(1-27)-MMP-2csV2-aCD3-aHER2-Xbody with MMP-2; 9) CD3ε(1-27)-FURiNcsV2-aCD3-aHER2-Xbody without MMP-2; 10) CD3ε(1-27)-FURINcsV2-aCD3-aHER2-Xbody with MMP-2; 11) CD3ε(1-27)-MMP-2csV3-aCD3-aHER2-Xbody without MMP-2; and 12) 11) CD3ε(1-27)-MMP-2csV3-aCD3-aHER2-Xbody with MMP-2. A “+” over a lane indicates samples treated with MMP2.

FIG. 7: Digestion of PABP and control molecules with MMP-2. Methods are described in Example 2, and the digestion products were run on an SOS-PAGE gel under reducing conditions. Lanes contain the following samples: 1) CD3ε(1-27)-aCD3-aHER2-mxb without MMP-2; 2) CD3ε(1-27)-aCD3-aHER2-mxb with MMP-2; 3) CD3ε(1-27)-MMP-2csV1-aCD3-aHER2-mxb without MMP-2; 4) CD3ε(1-27)-MMP-2csV1-aCD3-aHER2-mxb with MMP-2; 5) CD3ε(1-27)-MMP-2csV2-aCD3-aHER2-mxb without MMP-2; 6) CD3ε(1-27)-MMP-2csV2-aCD3-aHER2-Xbody with MMP-2; 7) CD3ε(1-27)-FURINcsV2-aCD3-aHER2-Xbody without MMP-2; and 8) CD3ε(1-27)-FURINcsV2-aCD3-aHER2-Xbody with MMP-2. A “+” over a lane indicates samples treated with MMP2.

FIG. 8: Digestion of PABP and control molecules with MMP-9. Methods are described in Example 2, and the digestion products were run on a SOS-PAGE gel under reducing conditions. Lanes contain the following samples: 1) CD3ε(1-27)-aCD3-aHER2-Xbody without MMP-2; 2) CD3ε(1-27)-aCD3-aHER2-Xbody with MMP-2; 3) CD3ε(1-27)-MMP-2csV1-aCD3-aHER2-Xbody without MMP-2; 4) CD3ε(1-27)-MMP-2csV1-aCD3-aHER2-Xbody with MMP-2; 5) CD3ε(1-27)-FURINcsV1-aCD3-aHER2-Xbody without MMP-2; 6) CD3ε(1-27)-FURINcsV1-aCD3-aHER2-Xbody with MMP-2; 7) CD3ε(1-27)-MMP-2csV2-aCD3-aHER2-Xbody without MMP-2; 8) CD3ε(1-27)-MMP-2csV2-aCD3-aHER2-Xbody with MMP-2; 9) CD3ε(1-27)-FURiNcsV2-aCD3-aHER2-Xbody without MMP-2; 10) CD3ε(1-27)-FURINcsV2-aCD3-aHER2-Xbody with MMP-2; 11) CD3ε(1-27)-MMP-2csV3-aCD3-aHER2-Xbody without MMP-2; 12) CD3ε(1-27)-MMP-2csV3-aCD3-aHER2-Xbody with MMP-2; 13) CD3ε(1-27)-aCD3-aHER2-mxb without MMP-2; 14) CD3(1-27)-aCD3-aHER2-mxb with MMP-2; 15) CD3ε(1-27)-MMP-2csV1-aCD3-aHER2-mxb without MMP-2; 16) CD3ε(1-27)-MMP-2csV1-aCD3-aHER2-mxb with MMP-2; 17) CD3ε(1-27)-MMP-2csV2-aCD3-aHER2-mxb without MMP-2; 18) CD3ε(1-27)-MMP-2csV2-aCD3-aHER2-mxb with MMP-2; 19) CD3ε(1-27)-FURINcsV2-aCD3-aHER2-mxb without MMP-2; and 20) CD3ε(1-27)-FURiNcsV2-aCD3-aHER2-mxb with MMP-2. A “+” over a lane indicates samples treated with MMP2.

FIG. 9A: Lysis of SKOV-3 cells in the presence of pan-T cells and control molecules. Methods are described in Example 3. The x axis represents the concentration of control molecule added to the assay, and the y axis represents the percent of cells lysed. Symbols signify data from assays done using the following proteins: filled circles with solid lines, aCD3-aHER2-Xbody; and filled squares with solid lines, aCD3-aHER2-mxb.

FIG. 9B: Percent of T cells expressing CD25. Methods are described in Example 3. The x axis represents the concentration of control molecule added to the assay, and the y axis represents the percent of cells expressing CD25. Symbols signify as in FIG. 9A.

FIG. 10A: Lysis of SKOV-3 cells in the presence of pan-T cells and PABPs or control molecules. Methods are described in Example 3. The x axis represents the concentration of PABP or control molecule added to the assay, and the y axis represents the percent of cells lysed. Symbols signify data from assays done using the following proteins: filled squares with solid lines, CD3ε(1-27)-aCD3-aHER2-Xbody, undigested; open squares with solid lines, CD3ε(1-27)-aCD3-aHER2-Xbody digested with MMP-2; filled triangles with solid lines, CD3ε(1-27)-MMP-2csV1-aCD3-aHER2-Xbody, undigested; open triangles with solid lines, CD3ε(1-27)-MMP-2csV1-aCD3-aHER2-Xbody, digested with MMP-2; filled circles with solid lines, CD3ε(1-27)-FURINcsV1-aCD3-aHER2-Xbody, undigested; and open circles with solid lines, CD3ε(1-27)-FURINcsV1-aCD3-aHER2-Xbody, digested with MMP-2.

FIG. 10B: Percent of T cells expressing CD25. Methods are described in Example 3. The x axis represents the concentration of control molecule or PABP added to the assay, and the y axis represents the percent of cells expressing CD25. Symbols signify as in FIG. 10B.

FIG. 11A: Lysis of SKOV-3 cells in the presence of pan-T cells and PABPs. Methods are described in Example 3. The x axis represents the concentration of PABP or control molecule added to the assay, and the y axis represents the percent of cells lysed. Symbols signify data from assays done using the following proteins: filled squares with solid lines, CD3ε(1-27)-MMP-2csV2-aCD3-aHER2-Xbody, undigested; open squares with solid lines, CD3ε(1-27)-MMP-2csV2-aCD3-aHER2-Xbody digested with MMP-2; filled triangles with solid lines, CD3ε(1-27)-FURINcsV2-aCD3-aHER2-Xbody, undigested; open triangles with solid lines, CD3ε(1-27)-FURINcsV2-aCD3-aHER2-Xbody, digested with MMP-2; filled circles with solid lines, CD3ε(1-27)-MMP-2csV3-aCD3-aHER2-Xbody, undigested; and open circles with solid lines, CD3ε(1-27)-MMP-2csV3-aCD3-aHER2-Xbody, digested with MMP-2.

FIG. 11B: Percent of T cells expressing CD25. Methods are described in Example 3. The x axis represents the concentration of control molecule or PABP added to the assay, and the y axis represents the percent of cells expressing CD25. Symbols signify as in FIG. 11B.

FIG. 12A: Lysis of SKOV-3 cells in the presence of pan-T cells and PABPs or control molecules. Methods are described in Example 3. The x axis represents the concentration of PABP or control molecule added to the assay, and the y axis represents the percent of cells lysed. Symbols signify data from assays done using the following proteins: filled squares with solid lines, CD3ε(1-27)-aCD3-aHER2-mxb, undigested; open squares with solid lines, CD3ε(1-27)-aCD3-aHER2-mxb, digested with MMP-2; filled, upward pointing triangles with solid lines, CD3ε(1-27)-MMP-2csV1-aCD3-aHER2-mxb, undigested; open, upward pointing triangles with solid lines, CD3ε(1-27)-MMP-2csV1-aCD3-aHER2-mxb, digested with MMP-2; filled circles with solid lines, CD3ε(1-27)-MMP-2csV2-aCD3-aHER2-mxb, undigested; open circles with solid lines, CD3ε(1-27)-MMP-2csV2-aCD3-aHER2-mxb, digested with MMP-2; filled diamonds with solid lines, CD3ε(1-27)-FURINcsV2-aCD3-aHER2-mxb; and open diamonds with solid lines, CD3ε(1-27)-FURINcsV2-aCD3-aHER2-mxb.

FIG. 12B: Percent of T cells expressing CD25. Methods are described in Example 3. The x axis represents the concentration of control molecule or PABP added to the assay, and the y axis represents the percent of cells expressing CD25. Symbols signify as in FIG. 12B.

FIG. 13: Lysis of JIMT-1 cells in the presence of pan-T cells and PABPs or control molecules. Methods are described in Example 5. The x axis indicated the concentration of the protein included in the assay (pM), and the y axis indicates the percent of the target cells (JIMT-1 cells) that were lysed..

### BRIEF DESCRIPTION OF THE SEQUENCE LISTING

| SEQ ID NO | DESCRIPTION OF SEQUENCE |
|---|---|
| 1 | Amino acid sequence of an MMP2 cleavage site |
| 2 | Amino acid sequence of an MMP2 cleavage site |
| 3 | Amino acid sequence of an MMP2 cleavage site |
| 4 | Amino acid sequence of a furin cleavage site |
| 5 | Amino acid sequence of a furin cleavage site |
| 6 | Amino acid sequence of the first polypeptide chain of CD3ε(1-27)-aCD3-aHER2-Xbody, CD3ε(1-27)-MMP-2csV1-aCD3-aHER2-Xbody, CD3ε(1-27)-MMP-2csV2-aCD3-aHER2-Xbody, CD3ε(1-27)-MMP-2csV3-aCD3-aHER2-Xbody, CD3ε(1-27)-FURINcsV1-aCD3-aHER2-Xbody, and CD3ε(1-27)-FURINcsV2-aCD3-aHER2-Xbody (including signal sequence) |
| 7 | Nucleic acid sequence encoding SEQ ID NO:6 |
| 8 | Amino acid sequence of the second polypeptide chain of CD3ε(1-27)-aCD3-aHER2-Xbody (including signal sequence) |
| 9 | Nucleic acid sequence encoding SEQ ID NO:8 |
| 10 | Amino acid sequence of the second polypeptide chain of CD3ε(1-27)-MMP-2csV1-aCD3-aHER2-Xbody (including signal sequence) |
| 11 | Nucleic acid sequence encoding SEQ ID NO:10 |
| 12 | Amino acid sequence of the second polypeptide chain of CD3ε(1-27)-MMP-2csV2-aCD3-aHER2-Xbody (including signal sequence) |
| 13 | Nucleic acid sequence encoding SEQ ID NO:12 |
| 14 | Amino acid sequence of the second polypeptide chain of CD3ε(1-27)-MMP-2csV3-aCD3-aHER2-Xbody (including signal sequence) |
| 15 | Nucleic acid sequence encoding SEQ ID NO:14 |
| 16 | Amino acid sequence of the second polypeptide chain of CD3ε(1-27)-FURINcsV1-aCD3-aHER2-Xbody (including signal sequence) |
| 17 | Nucleic acid sequence encoding SEQ ID NO:16 |
| 18 | Amino acid sequence of the second polypeptide chain of CD3ε(1-27)-FURINcsV2-aCD3-aHER2-Xbody (including signal sequence) |
| 19 | Nucleic acid sequence encoding SEQ ID NO:18 |
| 20 | Amino acid sequence of the first polypeptide chain of CD3ε(1-27)-aCD3-aHER2-mxb, CD3ε(1-27)-MMP-2csV1-aCD3-aHER2-mxb, CD3ε(1-27)-MMP-2csV2-aCD3-aHER2-mxb, and CD3ε(1-27)-FURINcsV2-aCD3-aHER2-mxb (including signal sequence) |
| 21 | Nucleic acid sequence encoding SEQ ID NO:20 |
| 22 | Amino acid sequence of the second polypeptide chain of CD3ε(1-27)-aCD3-aHER2-mxb (including signal sequence) |
| 23 | Nucleic acid sequence encoding SEQ ID NO:22 |
| 24 | Amino acid sequence of the second polypeptide chain of CD3ε(1-27)-MMP-2csV1-aCD3-aHER2-mxb (including signal sequence) |
| 25 | Nucleic acid sequence encoding SEQ ID NO:24 |
| 26 | Amino acid sequence of the second polypeptide chain of CD3ε(1-27)-MMP-2csV2-aCD3-aHER2-mxb (including signal sequence) |
| 27 | Nucleic acid sequence encoding SEQ ID NO:26 |
| 28 | Amino acid sequence of the second polypeptide chain of CD3ε(1-27)-FURINcsV2-aCD3-aHER2-mxb (including signal sequence) |
| 29 | Nucleic acid sequence encoding SEQ ID NO:28 |
| 30 | Amino acid sequence of the first polypeptide chain of aCD3-aHER2-Bi-Fc, CD3ε(1-27)-aCD3-aHER2-Bi-Fc, CD3ε(1-27)-MMP-2cs-aCD3-aHER2-Bi-Fc, and CD3ε(1-27)-FURINcs-aCD3-aHER2-Bi-Fc (including signal sequence) |
| 31 | Nucleic acid sequence encoding SEQ ID NO:30 |
| 32 | Amino acid sequence of the second polypeptide chain of aCD3-aHER2-Bi-Fc (including signal sequence) |
| 33 | Nucleic acid sequence encoding SEQ ID NO:32 |
| 34 | Amino acid sequence of the second polypeptide chain of CD3ε(1-27)-aCD3-aHER2-Bi-Fc (including signal sequence) |
| 35 | Nucleic acid sequence encoding SEQ ID NO:34 |
| 36 | Amino acid sequence of the second polypeptide chain of CD3ε(1-27)-MMP-2cs-aCD3-aHER2-Bi-Fc (including signal sequence) |
| 37 | Nucleic acid sequence encoding SEQ ID NO:36 |
| 38 | Amino acid sequence of the second polypeptide chain of CD3ε(1-27)-FURINcs-aCD3-aHER2-Bi-Fc (including signal sequence) |
| 39 | Nucleic acid sequence encoding SEQ ID NO:38 |
| 40 | Amino acid sequence of an anti-CD3ε VH region |
| 41 | Nucleic acid sequence encoding SEQ ID NO:40 |
| 42 | Amino acid sequence of a heavy chain CDR1 of SEQ ID NO:40 |
| 43 | Amino acid sequence of a heavy chain CDR2 of SEQ ID NO:40 |
| 44 | Amino acid sequence of a heavy chain CDR3 of SEQ ID NO:40 |
| 45 | Amino acid sequence of an anti-CD3ε VL region |
| 46 | Nucleic acid sequence encoding SEQ ID NO:45 |
| 47 | Amino acid sequence of a light chain CDR1 of SEQ ID NO:45 |
| 48 | Amino acid sequence of a light chain CDR2 of SEQ ID NO:45 |
| 49 | Amino acid sequence of a light chain CDR3 of SEQ ID NO:45 |
| 50 | Mature amino acid sequence of human CD3ε |
| 51 | Mature amino acid sequence of cynomolgus monkey CD3ε |
| 52 | Amino acid sequence of the extracellular domain of human CD3ε |
| 53 | Amino acids 1-27 of mature human CD3ε |
| 54 | Peptide sequence from human CD3ε |
| 55 | Amino acid sequence of a meprin α or β cleavage site |
| 56 | Amino acid sequence of a meprin α or β cleavage site |
| 57 | Amino acid sequence of a meprin α or β cleavage site |
| 58 | Amino acid sequence of a meprin α or β cleavage site |
| 59 | Amino acid sequence of a u-PA cleavage site |
| 60 | Amino acid sequence of a u-PA cleavage site |
| 61 | Amino acid sequence of a u-PA cleavage site |
| 62 | Amino acid sequence of a u-PA cleavage site |
| 63 | Amino acid sequence of a u-PA cleavage site |
| 64 | Amino acid sequence of a u-PA cleavage site |
| 65 | Amino acid sequence of a u-PA cleavage site |
| 66 | Amino acid sequence of a tPA cleavage site |
| 67 | Amino acid sequence of a cathepsin B cleavage site |
| 68 | Amino acid sequence of a cathepsin B cleavage site |
| 69 | Amino acid sequence of a cathepsin B cleavage site |
| 70 | Amino acid sequence of a cathepsin B cleavage site |
| 71 | Amino acid sequence of a cathepsin B cleavage site |
| 72 | Amino acid sequence of a cathepsin B cleavage site |
| 73 | Amino acid sequence of a cathepsin B cleavage site |
| 74 | Amino acid sequence of a cathepsin B cleavage site |
| 75 | Amino acid sequence of a cathepsin B cleavage site |
| 76 | Amino acid sequence of a cathepsin B cleavage site |
| 77 | Amino acid sequence of a cathepsin B cleavage site |
| 78 | Amino acid sequence of a cathepsin B cleavage site |
| 79 | Amino acid sequence of a cathepsin B cleavage site |
| 80 | Amino acid sequence of a cathepsin B cleavage site |
| 81 | Amino acid sequence of a cathepsin B cleavage site |
| 82 | Amino acid sequence of a furin cleavage site |
| 83 | Amino acid sequence of a fragment of human fibronectin |
| 84 | Amino acid sequence of a human IgG1 Fc polypeptide chain |
| 85 | Amino acid sequence of a human IgG2 Fc polypeptide chain |
| 86 | Amino acid sequence of a human IgG3 Fc polypeptide chain |
| 87 | Amino acid sequence of a human IgG4 Fc polypeptide chain |
| 88 | Amino acid sequence of a linker |
| 89 | Amino acid sequence of a linker |
| 90 | Amino acid sequence of a linker |
| 91 | Amino acid sequence of a linker |
| 92 | Amino acid sequence of a linker |
| 93 | Amino acid sequence of a second polypeptide chain of aCD3-aHER2-Xbody |
| 94 | Amino acid sequence of a second polypeptide chain of aCD3-aHER2-mxb |
| 95 | Amino acid sequence of a matrix metalloproteinase-11 (MMP-11) cleavage site |
| 96 | Amino acid sequence of an MMP-11 cleavage site |
| 97 | Amino acid sequence of an MMP-11 cleavage site |
| 98 | Amino acid sequence of an MMP-11 cleavage site |
| 99 | Amino acid sequence of an MMP-11 cleavage site |
| 100 | Amino acid sequence of an MMP-11 cleavage site |
| 101 | Amino acid insertion that extends half life of an Fc region |
| 102 | Amino acid insertion that extends half life of an Fc region |
| 103 | Amino acid insertion that extends half life of an Fc region |
| 104 | Amino acid insertion that extends half life of an Fc region |
| 105 | Amino acid insertion that extends half life of an Fc region |
| 106 | Amino acid insertion that extends half life of an Fc region |
| 107 | Amino acid insertion that extends half life of an Fc region |
| 108 | Amino acid insertion that extends half life of an Fc region |
| 109 | Amino acid insertion that extends half life of an Fc region |
| 110 | Amino acid insertion that extends half life of an Fc region |
| 111 | Amino acid insertion that extends half life of an Fc region |
| 112 | Amino acid insertion that extends half life of an Fc region |

### DETAILED DESCRIPTION

Described herein are a number of formats for bispecific proteins, optionally bispecific antibodies, that can be activated by proteolytic cleavage. These proteins are referred to herein as protease-activatable bispecific proteins (PABPs). PABPs can find use in disease states where one or more proteases are abundant in a localized disease microenvironment, for example, in various cancers, inflammatory diseases, fibrotic diseases, and neurodegenerative diseases such as Alzheimer’s disease. See, e.g., Broder and Becker-Pauly (2013), Biochem. J. 450: 253-264. In such a situation, the bispecific protein can be activated in the presence of disease cells, but not in their absence. Thus, a bispecific protein as described herein can be specifically activated in a disease microenvironment and be less active or inactive in other areas of the body.

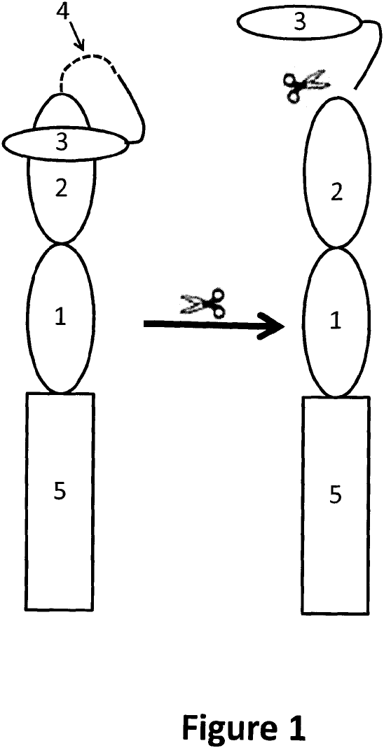

A PABP, which is diagrammed in FIG. 1, essentially contains three components and can contain two additional optional components. The various components of the molecule need not be ordered as in FIG. 1. Component 1 (oval labeled “1” in FIG. 1) can bind to a target molecule expressed on the surface of a pathogen, infected cell, or a cell that mediates a disease. Component 2 (oval labeled “2”) can bind to a effector cell molecule expressed on the surface of an effector cell that plays a role in cell killing, for example, a T cell. Component 3 (smaller oval labeled “3”), an optional component, can bind to Component 1 or 2, thereby blocking their binding to a target molecule or an effector cell molecule, respectively. Thus, for example, if Component 3 is bound to Component 2, the bispecific molecule is effectively monospecific or, at least less effective in binding a effector cell molecule. Some embodiments can lack Component 3, in which cases the binding Component 1 or Component 2 to a target or effector cell molecule, respectively, can be blocked or inhibited due to the three dimensional structure of the PABP. Component 4 (represented by a dashed line indicated by a “4”) is a linker comprising a protease cleavage site, which is located such that cleavage at this site allows binding of both Components 1 and 2 to their respective binding partners. In some embodiments, cleavage separates Component 3 from the remainder of the PABP, thereby activating the molecule, i.e., making it fully bispecific. In other embodiments, cleavage can make Component 1 or 2 more accessible and, thus, more active. In some embodiments the PABP can further comprise a Component 5 (rectangle labeled “5”) that extends half life. Component 5 can be, for example, an Fc polypeptide chain, all or part of a serum albumin protein, or other polypeptides that can extend in vivo half life.

### Definitions

An “antibody,” as meant herein, is a protein containing at least one immunoglobulin heavy chain variable region (VH) or light chain variable region (VL), in many cases a VH and a VL. Thus, the term “antibody” encompasses molecules having a variety of formats, including single chain Fv antibodies (scFv, which contain VH and VL regions joined by a linker), Fab, F(ab)₂′, Fab′, scFv:Fc antibodies (as described in Carayannopoulos and Capra, Ch. 9 in FUNDAMENTAL IMMUNOLOGY, 3^{rd} ed., Paul, ed., Raven Press, New York, 1993, pp. 284-286) or full length antibodies containing two full length heavy and two full length light chains, such as naturally-occurring IgG antibodies found in mammals. Id. Such full length antibodies, referred to herein as “IgG antibodies,” can be of the IgG1, IgG2, IgG3, or IgG4 isotype and can be human antibodies. The portions of Carayannopoulos and Capra that describe the structure of antibodies are incorporated herein by reference. Further, the term “antibody” includes dimeric antibodies containing two heavy chains and no light chains such as the naturally-occurring antibodies found in camels and other dromedary species and sharks. See, e.g., Muldermans et al., 2001, J. Biotechnol. 74:277-302; Desmyter et al., 2001, J. Biol. Chem. 276:26285-90; Streltsov et al. (2005), Protein Science 14: 2901-2909. An antibody can be “monospecific” (that is, binding to only one kind of antigen), “bispecific” (that is, binding to two different antigens), or “multispecific” (that is, binding to more than one different antigen). Further, an antibody can be monovalent, bivalent, or multivalent, meaning that it can bind to one, two, or multiple antigen molecules at once, respectively.

An “immunoglobulin heavy chain,” as meant herein, consists essentially of a VH, a first heavy chain constant region (CH1), a hinge region, a second heavy chain constant region (CH2), a third heavy chain constant region (CH3), in that order, and, optionally, a region downstream of the CH3 in some isotypes. Close variants of an immunoglobulin heavy chain containing no more than 10 amino acid substitutions, insertions, and/or deletions of a single amino acid per 100 amino acids relative to a known or naturally occurring immunoglobulin heavy chain amino acid sequence are encompassed within what is meant by an immunoglobulin heavy chain.

A “immunoglobulin light chain,” as meant herein, consists essentially of a VL and a light chain constant domain (CL). Close variants of an immunoglobulin light chain containing no more than 10 amino acid substitutions, insertions, and/or deletions of a single amino acid per 100 amino acids relative to a known or naturally occurring immunoglobulin light chain amino acid sequence are encompassed within what is meant by an immunoglobulin light chain.

An “immunoglobulin variable region,” as meant herein, is a VH, a VL, or a variant thereof. Close variants of an immunoglobulin variable region containing no more than 10 amino acid substitutions, insertions, and/or deletions of a single amino acid per 100 amino acids relative to a known or naturally occurring immunoglobulin variable region amino acid sequence are encompassed within what is meant by an immunoglobulin variable region. Many examples of VHs and VLs are known in the art, such as, for example, those disclosed by Kabat et al. in SEQUENCES OF IMMUNOLOGICAL INTEREST, Public Health Service N.I.H., Bethesda, MD, 1991. Based on the extensive sequence commonalities in the less variable portions of the VHs and VLs, the position within a sequence of more variable regions, and the predicted tertiary structure, one of skill in the art can recognize an immunoglobulin variable region by its sequence. See, e.g., Honegger and Pluckthun (2001), J. Mol. Biol. 309: 657-670.

An immunoglobulin variable region contains three hypervariable regions, known as complementarity determining region 1 (CDR1), complementarity determining region 2 (CDR2), and complementarity determining region 3 (CDR3). These regions form the antigen binding site of an antibody. The CDRs are embedded within the less variable framework regions (FR1-FR4). The order of these subregions within an immunoglobulin variable region is as follows: FR1-CDR1-FR2-CDR2-FR3-CDR3-FR4. Numerous sequences of immunoglobulin variable regions are known in the art. See, e.g., Kabat et al., SEQUENCES OF PROTEINS OF IMMUNOLOGICAL INTEREST, Public Health Service N.I.H., Bethesda, MD, 1991.

CDRs can be located in a VH region sequence in the following way. CDR1 starts at approximately residue 31 of the mature VH region and is usually about 5-7 amino acids long, and it is almost always preceded by a Cys-Xxx-Xxx-Xxx-Xxx-Xxx-Xxx-Xxx-Xxx (SEQ ID NO: ) (where “Xxx” is any amino acid). The residue following the heavy chain CDR1 is almost always a tryptophan, often a Trp-Val, a Trp-lle, or a Trp-Ala. Fourteen amino acids are almost always between the last residue in CDR1 and the first in CDR2, and CDR2 typically contains 16 to 19 amino acids. CDR2 may be immediately preceded by Leu-Glu-Trp-Ile-Gly (SEQ ID NO: ) and may be immediately followed by Lys/Arg-Leu/lle/Val/Phe/Thr/Ala-Thr/Ser/lle/Ala. Other amino acids may precede or follow CDR2. Thirty two amino acids are almost always between the last residue in CDR2 and the first in CDR3, and CDR3 can be from about 3 to 25 residues long. A Cys-Xxx-Xxx almost always immediately precedes CDR3, and a Trp-Gly-Xxx-Gly (SEQ ID NO: ) almost always follows CDR3.

Light chain CDRs can be located in a VL region in the following way. CDR1 starts at approximately residue 24 of the mature antibody and is usually about 10 to 17 residues long. It is almost always preceded by a Cys. There are almost always 15 amino acids between the last residue of CDR1 and the first residue of CDR2, and CDR2 is almost always 7 residues long. CDR2 is typically preceded by Ile-Tyr, Val-Tyr, Ile-Lys, or Ile-Phe. There are almost always 32 residues between CDR2 and CDR3, and CDR3 is usually about 7 to 10 amino acids long. CDR3 is almost always preceded by Cys and usually followed by Phe-Gly-Xxx-Gly (SEQ ID NO: ).

When a VH and/or VL, is said to “bind” to a target or immune effector cell “when it is part of an IgG and/or scFv antibody,” it is meant that an IgG or scFv antibody that contains the named VH and VL can bind to the target cell and/or the immune effector cell. The binding assay described in Example 5 can be used to assess binding.

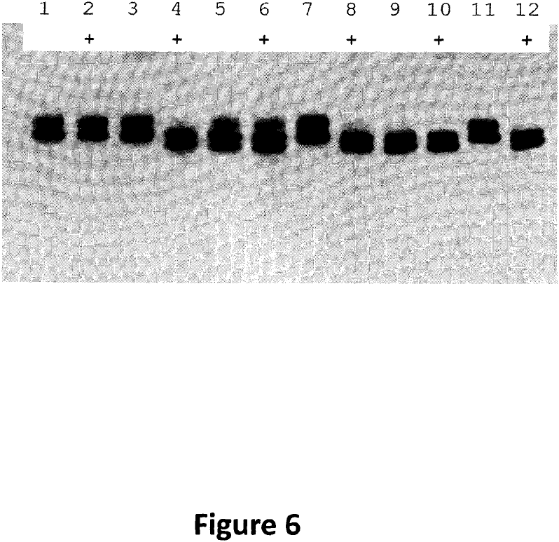

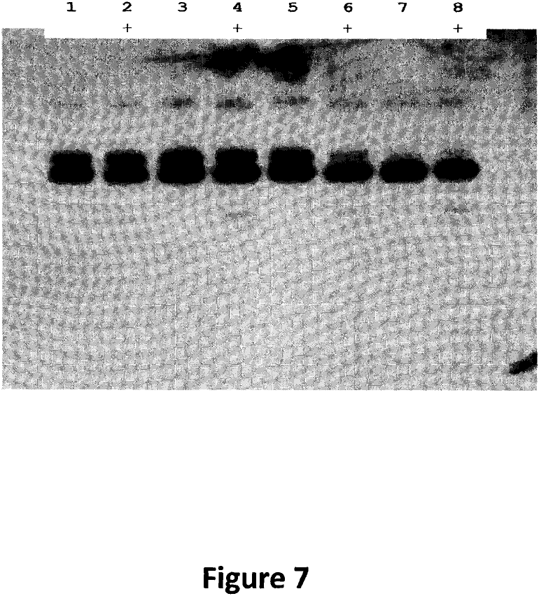

When a polypeptide is said to “inhibit the binding of polypeptide chain(s) to target or effector cells,” inhibition of binding is determined by binding assay using fluorescence-activated cell sorting (FACS) described in Example 5. Similarly, when it is said that “polypeptide chain(s) binds more effectively to a target or effector cell when a protease cleavage site is essentially completely cleaved,” the improvement in binding is assessed by the same assay. The essentially complete cleavage of a protease cleavage site is assessed by Western blot as explained in Example 2 and shown in FIGS. 6-8. For example, lanes 4, 8-10, and 12 in FIG. 6 show essentially complete cleavage since little, if any, of the upper band visible without digestion is detectable in these digested samples. Note that very minor amount of this upper band may possibly be present in lanes 4 and 8 of FIG. 6, but samples containing such small amounts of uncleaved species would be considered essentially completely cleaved as meant herein. In contrast, lanes 4 and 6 in FIG. 7 show partial cleavage. A lack of cleavage can be assessed by the same method. For example, lane 2 in FIG. 7 indicates a complete lack of cleavage since it looks essentially identical to lane 1, which was not digested with MMP2. Further, this same definition of essentially complete cleavage applies when it is said that “the Ec50 of the protein in a cell cytolysis assay when the protease cleavage site is essentially completely cleaved is less than a fifth of the Ec50 in the same assay when the protease cleavage site has not been cleaved.”

A “cancer cell antigen,” as meant herein, is a molecule, optionally a protein, expressed on the surface of a cancer cell. Some cancer cell antigens are also expressed on some normal cells, and some are specific to cancer cells. Cancer cell antigens can be highly expressed on the surface of a cancer cell. There are a wide variety of cancer cell antigens. Examples of cancer cell antigens include, without limitation, the following human proteins: epidermal growth factor receptor (EGFR), EGFRvIII (a mutant form of EGFR), melanoma-associated chondroitin sulfate proteoglycan (MCSP), mesothelin (MSLN), folate receptor 1 (FOLR1), CD33, CDH19, and epidermal growth factor 2 (HER2), among many others.

“Chemotherapy,” as used herein, means the treatment of a cancer patient with a “chemotherapeutic agent” that has cytotoxic or cytostatic effects on cancer cells. A “chemotherapeutic agent” specifically targets cells engaged in cell division and not cells that are not engaged in cell division. Chemotherapeutic agents directly interfere with processes that are intimately tied to cell division such as, for example, DNA replication, RNA synthesis, protein synthesis, the assembly, disassembly, or function of the mitotic spindle, and/or the synthesis or stability of molecules that play a role in these processes, such as nucleotides or amino acids. A chemotherapeutic agent therefore has cytotoxic or cytostatic effects on both cancer cells and other cells that are engaged in cell division. Chemotherapeutic agents are well-known in the art and include, for example: alkylating agents (e.g. busulfan, temozolomide, cyclophosphamide, lomustine (CCNU), methyllomustine, streptozotocin, cis-diamminedi-chloroplatinum, aziridinylbenzo-quinone, and thiotepa); inorganic ions (e.g. cisplatin and carboplatin); nitrogen mustards (e.g. melphalan hydrochloride, ifosfamide, chlorambucil, and mechlorethamine HCI); nitrosoureas (e.g. carmustine (BCNU)); anti-neoplastic antibiotics (e.g. adriamycin (doxorubicin), daunomycin, mitomycin C, daunorubicin, idarubicin, mithramycin, and bleomycin); plant derivatives (e.g. vincristine, vinblastine, vinorelbine, paclitaxel, docetaxel, vindesine, VP-16, and VM-26); antimetabolites (e.g. methotrexate with or without leucovorin, 5-fluorouracil with or without leucovorin, 5-fluorodeoxyuridine, 6-mercaptopurine, 6-thioguanine, cytarabine, 5-azacytidine, hydroxyurea, deoxycoformycin, gemcitabine, and fludarabine); podophyllotoxins (e.g. etoposide, irinotecan, and topotecan); as well as actinomycin D, dacarbazine (DTIC), mAMSA, procarbazine, hexamethylmelamine, pentamethylmelamine, L-asparaginase, and mitoxantrone, among many known in the art. See e.g. Cancer: Principles and Practice of Oncology, 4^{th} Edition, DeVita et al., eds., J.B. Lippincott Co., Philadelphia, PA (1993), the relevant portions of which are incorporated herein by reference. Alkylating agents and nitrogen mustard act by alkylating DNA, which restricts uncoiling and replication of strands. Methotrexate, cytarabine, 6-mercaptopurine, 5-fluorouracil, and gemcitabine interfere with nucleotide synthesis. Plant derivatives such a paclitaxel and vinblastine are mitotic spindle poisons. The podophyllotoxins inhibit topoisomerases, thus interfering with DNA replication. Antibiotics doxorubicin, bleomycin, and mitomycin interfere with DNA synthesis by intercalating between the bases of DNA (inhibiting uncoiling), causing strand breakage, and alkylating DNA, respectively. Other mechanisms of action include carbamoylation of amino acids (lomustine, carmustine) and depletion of asparagine pools (asparaginase). Merck Manual of Diagnosis and Therapy, 17^{th} Edition, Section 11, Hematology and Oncology, 144. Principles of Cancer Therapy, Table 144-2 (1999). Specifically included among chemotherapeutic agents are those listed above and those that directly affect the same cellular processes that are directly affected by the chemotherapeutic agents listed above.

A drug or treatment is “concurrently” administered with a PABP, as meant herein, if it is administered in the same general time frame as the PABP, optionally, on an ongoing basis. For example, if a patient is taking Drug A once a week on an ongoing basis and the PABP once every six months on an ongoing basis, Drug A and the PABP are concurrently administered, whether or not they are ever administered on the same day. Similarly, if the PABP is taken once per week on an ongoing basis and Drug A is administered only once or a few times on a daily basis, Drug A and the PABP are concurrently administered as meant herein. Similarly, if both Drug A and the PABP are administered for short periods of time either once or multiple times within a one month period, they are administered concurrently as meant herein as long as both drugs are administered within the same month.

A “conservative amino acid substitution,” as meant herein, is a substitution of an amino acid with another amino acid with similar properties. Properties considered include chemical properties such as charge and hydrophobicity. Table 1 below lists substitutions for each amino acid that are considered to be conservative substitutions as meant herein.

TABLE 1

Conservative Amino Acid Substitutions

| | |
|---|---|
| Original Residue | Conservative Substitutions |
| Ala | Val, Leu, lle |
| Arg | Lys, Gln, Asn |
| Asn | Gln |
| Asp | Glu |
| Cys | Ser, Ala |
| Gln | Asn |
| Glu | Asp |
| Gly | Pro, Ala |
| His | Asn, Gln, Lys, Arg |
| lle | Leu, Val, Met, Ala, Phe, Norleucine |
| Leu | Norleucine, lle, Val, Met, Ala, Phe |
| Lys | Arg, Gln, Asn |
| Met | Leu, Phe, lle |
| Phe | Leu, Val, lle, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | lle, Met, Leu, Phe, Ala, Norleucine |

An “effector cell,” as meant herein, is a cell that is involved in the mediation of a cytolytic immune response, including, for example, T cells, NK cells, monocytes, macrophages, or neutrophils. The protease-activatable bispecific antibodies described herein bind to a molecule that is expressed on the surface of an effector cell. Such proteins are referred to herein as “effector cell molecule.”

As meant herein, an “Fc region” is a dimer consisting of two polypeptide chains joined by one or more disulfide bonds, each chain comprising part or all of a hinge domain plus a CH2 and a CH3. Each of the polypeptide chains is referred to as an “Fc polypeptide chain.” To distinguish the two Fc polypeptide chains, in some instances one is referred to herein as an “A chain” and the other is referred to as a “B chain.” More specifically, the Fc regions contemplated for use with the present invention are IgG Fc regions, which can be mammalian, for example human, IgG1, IgG2, IgG3, or IgG4 Fc regions. Among human IgG1 Fc regions, at least two allelic types are known. In other embodiments, the amino acid sequences of the two Fc polypeptide chains can vary from those of a mammalian Fc polypeptide by no more than 10 substitutions, insertions, and/or deletions of a single amino acid per 100 amino acids of sequence relative to a mammalian Fc polypeptide amino acid sequence. In some embodiments, such variations can be “heterodimerizing alterations” that facilitate the formation of heterodimers over homodimers, an Fc alteration that extends half life, an alteration that inhibits Fc gamma receptor (FcyR) binding, and/or an alteration that enhances Fcy receptor binding and enhances ADCC.

An “Fc alteration that extends half life,” as meant herein is an alteration within an Fc polypeptide chain that lengthens the in vivo half life of a protein that contains the altered Fc polypeptide chain as compared to the half life of a similar protein containing the same Fc polypeptide, except that it does not contain the alteration. Such alterations can be included in an Fc polypeptide chain that is part of a PABP as described herein. The alterations M252Y, S254T, and T256E (methionine at position 252 changed to tyrosine; serine at position 254 changed to threonine; and threonine at position 256 changed to glutamic acid; numbering according to EU numbering as shown in Table 2) are Fc alterations that extend half life and can be used together, separately or in any combination. These alterations and a number of others are described in detail in U.S. Pat. 7,083,784. The portions of U.S. Pat. 7,083,784 that describe such alterations are incorporated herein by reference. Similarly, M428L and N434S are Fc alterations that extend half life and can be used together, separately or in any combination. These alterations and a number of others are described in detail in U.S. Pat. Application Publication 2010/0234575 and U.S. Pat. 7,670,600. The portions of U.S. Pat. Application Publication 2010/0234575 and U.S. Pat. 7,670,600 that describe such alterations are incorporated herein by reference. In addition, any substitution at one of the following sites can be considered an Fc alteration that extends half life as meant here: 250, 251, 252, 259, 307, 308, 332, 378, 380, 428, 430, 434, 436. Each of these alterations or combinations of these alterations can be used to extend the half life of a PABP as described herein. Other alterations that can be used to extend half life are described in detail in International Application PCT/US2012/070146 filed Dec. 17, 2012. The portions of this application that describe such alterations are incorporated herein by reference. Some specific embodiments described in this application include insertions between positions 384 and 385 (EU numbering as shown in Table 2) that extend half life, including the following amino acid sequences: GGCVFNMFNCGG (SEQ ID NO:101), GGCHLPFAVCGG (SEQ ID NO:102), GGCGHEYMWCGG (SEQ ID NO:103), GGCWPLQOYCGG(SEQ ID NO:104), GGCMQMNKWCGG (SEQ ID NO:105), GGCDGRTKYCGG (SEQ ID NO:106), GGCALYPTNCGG (SEQ ID NO:107), GGCGKHWHQCGG (SEQ ID NO:108), GGCHSFKHFCGG (SEQ ID NO:109), GGCQGMWTWCGG (SEQ ID NO:110), GGCAQQWHHEYCGG (SEQ ID NO:111), and GGCERFHHACGG (SEQ ID NO:112), among others. PABPs containing such insertions are contemplated.

A “half life-extending moiety,” as meant herein, is a molecule that extends the in vivo half life of a protein to which it is attached as compared to the in vivo half life of the protein without the half life-extending moiety. Methods for measuring half life are well known in the art. A method for ascertaining half life is disclosed, for example, in WO 2013/096221, the relevant portions of which are incorporated herein by reference. Essentially, the molecule is administered to an animal or a human at a known dosage and amounts of the molecule in blood are assayed over time post-dose. A half life-extending moiety can be a polypeptide, for example an Fc polypeptide chain or a polypeptide that can bind to albumin. The amino acid sequence of a domain of human fibronectin type III (Fn3) that has been engineered to bind to albumin is provided in SEQ ID NO:83, and various human IgG Fc polypeptide sequences are given in SEQ ID NOs:84-87. An Fc polypeptide can, for example, be modified so that it is more effective at extending half life than an unmodified Fc polypeptide chain. Such modifications include, for example, those described above as “Fc alterations that extend half life.” In alternate embodiments, a half life-extending moiety can be a non-polypeptide molecule. For example, a polyethylene glycol (PEG) molecule can be a half life-extending moiety. Other half-life extending moieties, including a variety of polypeptides, are contemplated.

A “heterodimer,” as meant herein, is a dimer comprising two polypeptide chains with different amino acid sequences.

“Heterodimerizing alterations” generally refer to alterations in the A and B chains of an Fc region that facilitate the formation of heterodimeric Fc regions, that is, Fc regions in which the A chain and the B chain of the Fc region do not have identical amino acid sequences. Such alterations can be included in an Fc polypeptide chain that is part of a PABP as described herein. Heterodimerizing alterations can be asymmetric, that is, an A chain having a certain alteration can pair with a B chain having a different alteration. These alterations facilitate heterodimerization and disfavor homodimerization. Whether hetero- or homo-dimers have formed can be assessed by size differences as determined by polyacrylamide gel electrophoresis in some situations or by other appropriate means such as differing charges or biophysical characteristics, including binding by antibodies or other molecules that recognize certain portions of the heterodimer including molecular tags. One example of such paired heterodimerizing alterations are the so-called “knobs and holes” substitutions. See, e.g., U.S. Pat. 7,695,936 and U.S. Pat. Application Publication 2003/0078385, the portions of which describe such mutations are incorporated herein by reference. As meant herein, an Fc region that contains one pair of knobs and holes substitutions, contains one substitution in the A chain and another in the B chain. For example, the following knobs and holes substitutions in the A and B chains of an IgG1 Fc region have been found to increase heterodimer formation as compared with that found with unmodified A and B chains: 1) Y407T in one chain and T366Y in the other; 2) Y407A in one chain and T366W in the other; 3) F405A in one chain and T394W in the other; 4) F405W in one chain and T394S in the other; 5) Y407T in one chain and T366Y in the other; 6) T366Y and F405A in one chain and T394W and Y407T in the other; 7) T366W and F405W in one chain and T394S and Y407A in the other; 8) F405W and Y407A in one chain and T366W and T394S in the other; and 9) T366W in one polypeptide of the Fc and T366S, L368A, and Y407V in the other. This way of notating mutations can be explained as follows. The amino acid (using the one letter code) normally present at a given position in the CH3 region using the EU numbering system (which is presented in Edelman et al. (1969), Proc. Natl. Acad. Sci. 63: 78-85; see also Table 2 below) is followed by the EU position, which is followed by the alternate amino acid that is present at that position. For example, Y407T means that the tyrosine normally present at EU position 407 is replaced by a threonine. Alternatively or in addition to such alterations, substitutions creating new disulfide bridges can facilitate heterodimer formation. See, e.g., U.S. Pat. Application Publication 2003/0078385, the portions of which describe such mutations are incorporated herein by reference. Such alterations in an IgG1 Fc region include, for example, the following substitutions: Y349C in one Fc polypeptide chain and S354C in the other; Y349C in one Fc polypeptide chain and E356C in the other; Y349C in one Fc polypeptide chain and E357C in the other; L351C in one Fc polypeptide chain and S354C in the other; T394C in one Fc polypeptide chain and E397C in the other; or D399C in one Fc polypeptide chain and K392C in the other. Similarly, substitutions changing the charge of a one or more residue, for example, in the C_{H}3-C_{H}3 interface, can enhance heterodimer formation as explained in WO 2009/089004, the portions of which describe such substitutions are incorporated herein by reference. Such substitutions are referred to herein as “charge pair substitutions,” and an Fc region containing one pair of charge pair substitutions contains one substitution in the A chain and a different substitution in the B chain. General examples of charge pair substitutions include the following: 1) K409D or K409E in one chain plus D399K or D399R in the other; 2) K392D or K392E in one chain plus D399K or D399R in the other; 3) K439D or K439E in one chain plus E356K or E356R in the other; and 4) K370D or K370E in one chain plus E357K or E357R in the other. In addition, the substitutions R355D, R355E, K360D, or K360R in both chains can stabilize heterodimers when used with other heterodimerizing alterations. Specific charge pair substitutions can be used either alone or with other charge pair substitutions. Specific examples of single pairs of charge pair substitutions and combinations thereof include the following: 1) K409E in one chain plus D399K in the other; 2) K409E in one chain plus D399R in the other; 3) K409D in one chain plus D399K in the other; 4) K409D in one chain plus D399R in the other; 5) K392E in one chain plus D399R in the other; 6) K392E in one chain plus D399K in the other; 7) K392D in one chain plus D399R in the other; 8) K392D in one chain plus D399K in the other; 9) K409D and K360D in one chain plus D399K and E356K in the other; 10) K409D and K370D in one chain plus D399K and E357K in the other; 11) K409D and K392D in one chain plus D399K, E356K, and E357K in the other; 12) K409D and K392D on one chain and D399K on the other; 13) K409D and K392D on one chain plus D399K and E356K on the other; 14) K409D and K392D on one chain plus D399K and D357K on the other; 15) K409D and K370D on one chain plus D399K and D357K on the other; 16) D399K on one chain plus K409D and K360D on the other; and 17) K409D and K439D on one chain plus D399K and E356K on the other. Any of the these heterodimerizing alterations can be used in the Fc regions of the heterodimeric bispecific antibodies described herein.

An “alteration that inhibits FcyR binding,” as meant herein, is one or more insertions, deletions, or substitutions within an Fc polypeptide chain that inhibits the binding of FcyRIIA, FcyRIIB, and/or FcyRlllA as measured, for example, by an ALPHALISA®-based competition binding assay (PerkinElmer, Waltham, MA). Such alterations can be included in an Fc polypeptide chain that is part of a PABP as described herein. More specifically, alterations that inhibit Fc gamma receptor (FcyR) binding include L234A, L235A, or any alteration that inhibits glycosylation at N297, including any substitution at N297. In addition, along with alterations that inhibit glycosylation at N297, additional alterations that stabilize a dimeric Fc region by creating additional disulfide bridges are also contemplated. Further examples of alterations that inhibit FcyR binding include a D265A alteration in one Fc polypeptide chain and an A327Q alteration in the other Fc polypeptide chain.

An “alteration that enhances ADCC,” as meant herein is one or more insertions, deletions, or substitutions within an Fc polypeptide chain that enhances antibody dependent cell-mediated cytotoxicity (ADCC). Such alterations can be included in an Fc polypeptide chain that is part of a PABP as described herein. Many such alterations are described in International Patent Application Publication WO 2012/125850. Portions of this application that describe such alterations are incorporated herein by reference. Such alterations can be included in an Fc polypeptide chain that is part of a PABP as described herein. ADCC assays can be performed as follows. Cell lines that express high and lower amounts of a cancer cell antigen on the cell surface can be used as target cells. These target cells can belabeled with carboxyfluorescein succinimidyl ester (CFSE) and then washed once with phosphate buffered saline (PBS) before being deposited into 96-well microtiter plates with V-shaped wells. Purified immune effector cells, for example T cells, NK cells, macrophages, monocytes, or peripheral blood mononuclear cells (PBMCs), can be added to each well. A monospecific antibody that binds to the cancer antigen and contains the alteration(s) being tested and an isotype-matched control antibody can be diluted in a 1:3 series and added to the wells. The cells can be incubated at 37° C. with 5% CO₂ for 3.5 hrs. The cells can be spun down and re-suspended in 1x FACS buffer (1x phosphate buffered saline (PBS) containing 0.5% fetal bovine serum (FBS)) with the dye TO-PRO®-3iodide (Molecular Probes, Inc. Corporation, Oregon, USA), which stains dead cells, before analysis by fluorescence activated cell sorting (FACS). The percentage of cell killing can be calculated using the following formula:
(percent tumor cell lysis with bispecific - percent tumor cell lysis without bispecific)/
(percent total cell lysis - percent tumor cell lysis without bispecific)

Total cell lysis is determined by lysing samples containing effector cells and labeled target cells without a bispecific molecule with cold 80% methanol. Exemplary alterations that enhance ADCC include the following alterations in the A and B chains of anFc region: (a) the A chain comprises Q311M and K334V substitutions and the B chain comprises L234Y, E294L, and Y296W substitutions or vice versa; (b) the A chain comprises E233L, Q311M, and K334V substitutions and the B chain comprises L234Y, E294L, and Y296W substitutions or vice versa; (c) the A chain comprises L2341, Q311M, and K334V substitutions and the B chain comprises L234Y, E294L, and Y296W substitutions or vice versa; (d) the A chain comprises S298T and K334V substitutions and the B chain comprises L234Y, K290Y, and Y296W substitutions or vice versa; (e) the A chain comprises A330M and K334V substitutions and the B chain comprises L234Y, K290Y, and Y296W substitutions or vice versa; (f) the A chain comprises A330F and K334V substitutions and the B chain comprises L234Y, K290Y, and Y296W substitutions or vice versa; (g) the A chain comprises Q311M, A330M, and K334V substitutions and the B chain comprises L234Y, E294L, and Y296W substitutions or vice versa; (h) the A chain comprises Q311M, A330F, and K334V substitutions and the B chain comprises L234Y, E294L, and Y296W substitutions or vice versa; (i) the A chain comprises S298T, A330M, and K334V substitutions and the B chain comprises L234Y, K290Y, and Y296W substitutions or vice versa; (j) the A chain comprises S298T, A330F, and K334V substitutions and the B chain comprises L234Y, K290Y, and Y296W substitutions or vice versa; (k) the A chain comprises S239D, A330M, and K334V substitutions and the B chain comprises L234Y, K290Y, and Y296W substitutions or vice versa; (I) the A chain comprises S239D, S298T, and K334V substitutions and the B chain comprises L234Y, K290Y, and Y296W substitutions or vice versa; (m) the A chain comprises a K334V substitution and the B chain comprises Y296W and S298C substitutions or vice versa; (n) the A chain comprises a K334V substitution and the B chain comprises L234Y, Y296W, and S298C substitutions or vice versa; (o) the A chain comprises L235S, S239D, and K334V substitutions and the B chain comprises L234Y, K290Y, and Y296W, substitutions or vice versa; (p) the A chain comprises L235S, S239D, and K334V substitutions and the B chain comprises L234Y, Y296W, and S298C substitutions or vice versa; (q) the A chain comprises Q311M and K334V substitutions and the B chain comprises L234Y, F243V, and Y296W substitutions or vice versa; (r) the A chain comprises Q311M and K334V substitutions and the B chain comprises L234Y, K296W, and S298C substitutions or vice versa; (s) the A chain comprises S239D and K334V substitutions and the B chain comprises L234Y, K290Y, and Y296W substitutions or vice versa; (t) the A chain comprises S239D and K334V substitutions and the B chain comprises L234Y, Y296W, and S298C substitutions or vice versa; (u) the A chain comprises F243V and K334V substitutions and the B chain comprises L234Y, K290Y, and Y296W, substitutions or vice versa; (v) the A chain comprises F243V and K334V substitutions and the B chain comprises L234Y, Y296W, and S298C substitutions or vice versa; (w) the A chain comprises E294L and K334V substitutions and the B chain comprises L234Y, K290Y, and Y296W substitutions or vice versa; (x) the A chain comprises E294L and K334V substitutions and the B chain comprises L234Y, Y296W, and S298C substitutions or vice versa; (y) the A chain comprises A330M and K334V substitutions and the B chain comprises L234Y and Y296W substitutions or vice versa; or (z) the A chain comprises A330M and K334V substitutions and the B chain comprises K290Y and Y296W substitutions or vice versa.

A “linker,” as meant herein, is a peptide that links two polypeptides, which can, for example, be two immunoglobulin variable regions in the context of a PABP. A linker can be from 2-30 amino acids in length. In some embodiments, a linker can be 2-40, 2-40, or 3-18 amino acids long. In some embodiments, a linker can be a peptide no more than 40, 30, 20, 14, 13, 12, 11, 10, 9, 8, 7, 6, or 5 amino acids long. In other embodiments, a linker can be 5-40, 5-15, 4-11, 10-20, or 20-40 amino acids long. In other embodiments, a linker can be about, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids long. Exemplary linkers include, for example, the amino acid sequences (GGGGS)ₙ (where n is any integer from 1 to 10; SEQ ID NO:88), TVAAP (SEQ ID NO:89), ASTKGP (SEQ ID NO:90), GGGGSAAA (SEQ ID NO:91), GGGGSGGGGSGGGGS (SEQ ID NO:92), and AAA, among many others.

A PABP that “mediates cytolysis of a target cell by an immune effector cell,” as meant herein, when addition of an amount from 0.001 pM to 20000 pM of the PABP to a cell cytolysis assay as described herein effectively elicits cytolysis of of the target cells. A cytolysis assay is described in Example 3.

“Non-chemotherapeutic anti-neoplastic agents” are chemical agents, compounds, or molecules having cytotoxic or cytostatic effects on cancer cells other than chemotherapeutic agents. Non-chemotherapeutic antineoplastic agents may, however, be targeted to interact directly with molecules that indirectly affect cell division such as cell surface receptors, including receptors for hormones or growth factors. However, non-chemotherapeutic antineoplastic agents do not interfere directly with processes that are intimately linked to cell division such as, for example, DNA replication, RNA synthesis, protein synthesis, or mitotic spindle function, assembly, or disassembly. Examples of non-chemotherapeutic anti-neoplastic agents include inhibitors of Bcl2, inhibitors of farnesyltransferase, anti-estrogenic agents such as tamoxifen, anti-androgenic compounds, interferon, arsenic, retinoic acid, retinoic acid derivatives, antibodies targeted to tumor-specific antigens, and inhibitors of the Bcr-Abl tyrosine kinase (e.g., the small molecule STI-571 marketed under the trade name GLEEVEC™ by Novartis, New York and New Jersey, USA and Basel, Switzerland), among many possible non-chemotherapeutic anti-neoplastic agents.

A “non-cleavable linker,” as meant herein, is a linker that does not contain a protease cleavage site.

A “protease cleavage site,” as meant herein, includes an amino acid sequence that is cleaved by a protease, including all cleavage sites explicitly disclosed herein (in Table 2), as well as any others.

A “protein,” as meant herein, comprises a polypeptide chain of at least 30 amino acids joined by peptide bonds and can comprise multiple polypeptide chains. A protein can further comprise additional moieties added via post-tranlational modification, such as, for example, sugars.

A “target cell” is a cell that a PABP, as described herein, binds to and that is involved in mediating a disease. In some cases, a target cell can be a cell that is ordinarily involved in mediating an immune response, but is also involved in the mediation of a disease. For example in B cell lymphoma, a B cell, which is ordinarily involved in mediating immune response, can be a target cell. In some embodiments, a target cell is a cancer cell, a cell infected with a pathogen, or a cell involved in mediating an autoimmune or inflammatory disease. The PABP can bind to the target cell via binding to a “target molecule,” which can be, e.g., a protein or a sugar, which is displayed on the surface of the target cell, possibly a highly expressed protein or a protein with a restricted pattern of expression that is enriched in the target cell versus other kinds of cells or tissues in the body. A target molecule could also be, for example, a specific kind of sugar molecule.

A “therapeutically effective amount” of a PABP as described herein is an amount that has the effect of, for example, reducing or eliminating the tumor burden of a cancer patient or reducing or eliminating the symptoms of any disease condition that the protein is used to treat. A therapeutically effective amount need not completely eliminate all symptoms of the condition, but may reduce severity of one or more symptoms or delay the onset of more serious symptoms or a more serious disease that can occur with some frequency following the treated condition.

“Treatment” of any disease mentioned herein encompasses an alleviation of at least one symptom of the disease, a reduction in the severity of the disease, or the delay or prevention of disease progression to more serious symptoms that may, in some cases, accompany the disease or lead to at least one other disease. Treatment need not mean that the disease is totally cured. A useful therapeutic agent needs only to reduce the severity of a disease, reduce the severity of one or more symptoms associated with the disease or its treatment, or delay the onset of more serious symptoms or a more serious disease that can occur with some frequency following the treated condition.

When it is said that a named VH/VL pair of immunoglobulin variable regions can bind to a target cell or an immune effector cell “when they are part of an IgG or scFv antibody,” it is meant that an IgG antibody that contains the named VH region in both heavy chains and the named VL region in both light chains or an scFv that contains the VH/VL pair can bind to the target cell or the immune effector cell. A binding assay is described in Example 5. One of skill in the art could construct an IgG or scFv antibody containing the desired sequences given the knowledge in the art.

### Component 1 and Target Molecules

As explained above, Component 1 of a PABP is part of the PABP that can bind to a target molecule expressed the surface of the pathogen or an endogenous disease-mediating cell. A pathogen can be, for example, a virus, a bacterium, or a protozoan. In some embodiments, Component 1 comprises a heavy and a light chain variable (VH and VL) region that, together, can bind to the target molecule. The VH and VL regions can be on the same or different polypeptide chains. In other embodiments, Component 1 can be a VH or a VL region, as long as the VH or VL region can, alone, bind to the disease-mediating cell or pathogen. Such single variable domain antibodies are described in, for example, US 2008/0008713, the relevant portions of which are incorporated herein by reference. Any of these VH and/or VL regions can be of mammalian origin, for example, human VH and/or VL regions. In other embodiments, Component 1 can be a polypeptide that is not part of an antibody. For example, where the target molecule is mesothelin, Component 1 can be all or part of a polypeptide that binds to mesothelin or a short peptide selected by virtue of its ability to bind mesothelin.

The cell or pathogen that mediates a disease can express a target molecule on its surface. Such cells include, for example, endogenous cells that mediate a cancer, an autoimmune or inflammatory disease, a fibrotic disease, a neurodegenerative disease, or an infectious disease. For example, many proteins are known to be specifically expressed at high levels on cancer cells, on cells that mediate an autoimmune or inflammatory condition, or on infectious agents or infected cells. Such proteins are potential target molecules for PABPs described herein.

As explained above, a PABP, as described herein, binds to an effector cell molecule and a target molecule. The target molecule can, for example, be expressed on the surface of a cancer cell (i.e., a cancer cell antigen), a cell infected with a pathogen, or a cell that mediates an inflammatory, autoimmune, or fibrotic condition. In some embodiments, the target molecule can be highly expressed on the target cell, although this is not required.

Where the target cell is a cancer cell, a PABP can bind to a cancer cell antigen, as defined herein above. A cancer cell antigen can be a human protein and/or a protein from another species. For example, a PABP may bind to a target molecule, which can be a protein, from a mouse, rat, rabbit, new world monkey, and/or old world monkey species, among many others. Such species include, without limitation, the following species: Homo sapiens, Musmusculus;Rattusrattus;Rattusnorvegicus; cynomolgus monkey, Macacafascicularis; the hamadryas baboon, Papiohamadryas; the Guinea baboon, Papiopapio; the olive baboon, Papioanubis; the yellow baboon, Papiocynocephalus; the Chacma baboon, Papioursinus,Callifhrixjacchus,Saguinusoedipus, and Saimirisciureus.

In some examples, the target molecule can be a protein selectively expressed on an infected cell. For example, in the case of a hepatitis B virus (HBV) or hepatitis C virus (HCV) infection, the target molecule can be an envelope protein of HBV or HCV that is expressed on the surface of an infected cell. In other embodiments, the target molecule can be gp120 encoded by human immunodeficiency virus (HIV) expressed on HIV-infected cells. Similarly, the target molecule can be a molecule expressed on the surface of a pathogen including, for example, viruses, bacteria (including the species Borrelia, Staphylococcus, Escherichia, among many other species), fungi (including yeast), giardia, amoeba, eukarytic protists of the genus Plasmodium, ciliates, trypanosomes, nematodes, and other eukaryotic parasites.

In a condition where it is desirable to deplete regulatory T cells, such as in a cancer or an infectious disease, regulatory T cells can be target cells. If so, CCR4 can be a target molecule.

In other aspects, a target cell can be a cell that mediates an autoimmune or inflammatory disease. For example, human eosinophils in asthma can be target cells, in which case, EGF-like module containing, mucin-like hormone receptor 1 (EMR1), for example, can be a target molecule. Alternatively, excess human B cells in a systemic lupus erythematosus patient can be target cells, in which case CD19 or CD20, for example, can be a target molecule. In other autoimmune conditions, excess human Th2 T cells can be target cells, in which case CCR4 can, for example, be a target molecule. Similarly, a target cell can be a fibrotic cell that mediates a disease such as atherosclerosis, chronic obstructive pulmonary disease (COPD), cirrhosis, scleroderma, kidney transplant fibrosis, kidney allograft nephropathy, or a pulmonary fibrosis, including idiopathic pulmonary fibrosis and/or idiotypic pulmonary hypertension. For such fibrotic conditions, fibroblast activation protein alpha (FAP alpha) can, for example, be a target molecule.

Specific examples of Component 1 include, for example, VHNL pairs that bind to cancer cell antigens, e.g., a VH/VL pair comprising the amino acid sequences of amino acids 20-140 of SEQ ID NO:6 and amino acids 197-303 of SEQ ID NO:8.

### Component 2 and Effector Cell Molecules

Component 2 can bind to an effector cell molecule. It can comprise a VH and a VL region. In some embodiments, Component 2 can comprise a VH or a VL region, which, alone, can bind to the effector cell molecule. Any of these VH and/or VL regions can be of mammalian origin, for example, human VH and/or VL regions. Alternatively, Component 2 can be a non-antibody polypeptide that can bind to an effector cell molecule. Component 2 can bind to a molecule, which can be a protein, expressed on the surface of an effector cell. The effector cell can be, for example, a T cell, an NK cell, a monocyte, a macrophage, or a neutrophil.

In some embodiments the effector cell molecule is a protein included in a T cell receptor (TCR)-CD3 complex. There are at least three kinds of TCRs. An αβTCR complex contains a heterodimer consisting of TCRa and TCRβ (αβTCR), a homodimer consisting of two CD3ζ proteins (CD3ζζ), a heterodimer consisting of CD3δ and CD3ε (CD3δε), and a heterodimer consisting of CD3y and CD3ε (CD3yε). A yδTCR complex contains a heterodimer consisting of TCRy and TCRδ (yδTCR), plus CD3δε and CD3yε heterodimers and a CD3ζζ homodimer. A pTCR consists of a heterodimer consisting of pTα and TCRβ, plus CD3δε and CD3yε heterodimers and a CD3ζζ homodimer. See, e.g., Kuhns and Badgandi (2012), Immunological Rev. 250: 120-143, the relevant portions of which are incorporated by reference herein. Component 2 may bind to any of the proteins included in a TCR-CD3 complex.

In some embodiments, a PABP can bind to a human CD3ε chain (the mature amino acid sequence of which is disclosed in SEQ ID NO:50), which may be part of a multimeric protein. Alternatively, the effector cell molecule can be a human and/or cynomolgus monkey TCRa, TCRβ, TCRδ, TCRy, CD3β, CD3y, CD3δ, or CD3ζ.

In some embodiments, the PABP can bind to a CD3ε chain from another species, such as mouse, rat, rabbit, new world monkey, and/or old world monkey species. Such species include, without limitation, the following mammalian species: Musmusculus;Rattusrattus;Rattusnorvegicus; the cynomolgus monkey, Macacafascicularis; the hamadryas baboon, Papiohamadryas; the Guinea baboon, Papiopapio; the olive baboon, Papioanubis; the yellow baboon, Papiocynocephalus; the Chacma baboon, Papioursinus;Callithrixjacchus;SaguinusOedipus; and Saimirisciureus. The mature amino acid sequence of the CD3ε chain of cynomolgus monkey is provided in SEQ ID NO:51. As is known in the art of development of protein therapeutics, having a therapeutic that can have comparable activity in humans and species commonly used for preclinical testing, such as mice and monkeys, can simplify and speed drug development. In the long and expensive process of bringing a drug to market, such advantages can be critical.

In more particular embodiments, the PABP can bind to an epitope within the first 27 amino acids of the CD3ε chain, which may be a human CD3ε chain or a CD3ε chain from a different species, particularly one of the mammalian species listed above. The epitope that the antibody binds to can be part of an amino acid sequence selected from the group consisting of SEQ ID NO:52 and SEQ ID NO:53. The epitope can contain the amino acid sequence Gln-Asp-Gly-Asn-Glu (SEQ ID NO:54). The advantages of a protein that binds to this amino acid sequence are explained in detail in U.S. Pat. Application Publication 2010/183615, the relevant portions of which are incorporated herein by reference. The portion of a protein bound by an antibody or a protein can be determined by alanine scanning, which is described in, e.g., U.S. Pat. Application Publication 2010/183615, the relevant portions of which are incorporated herein by reference.

Where an NK cell or a cytotoxic T cell is an immune effector cell, NKG2D, CD352, NKp46, or CD16a can be an effector cell molecule to which Component 2 can bind. Where a CD8+ T cell is an immune effector cell, 4-1BB, OX40, GITR, CD28, CD27, or ICOS can be an effector cell molecule to which Component 2 can bind. Alternatively, a PABP could bind to other antigens expressed on T cells, NK cells, macrophages, monocytes, or neutrophils.

VH and VL regions that can be used as a Component 2 of a PABP include those that can can bind to CD3ε or other components of a TCR-CD3 complex, e.g., those comprising the amino acid sequences of SEQ ID NOs: 40 and 45. Other VH/VL pairs that can bind to CD3ε or other effector cell molecules expressed on T cells, NK cells, macrophages, monocytes, or neutrophils can also be used as a Component 2.

### Component 3

Component 3, an optional component, is a polypeptide that can bind to Component 1 or 2 and, when bound, can block or inhibit the binding of Component 1 or 2 to an effector cell or a target cell. In some embodiments, Component 3 is part or all of the target molecule to which Component 1 can bind or the effector cell molecule to which Component 2 can bind. For example, where the effector cell is a T cell, Component 3 can be part or all of a polypeptide that is part of the TCR-CD3 complex, such as TCRa, TCRβ, TCRδ, TCRy, pTa, CD3β, CD3y, CD3δ, CD3ε, or CD3ζ. Alternatively, where the effector cell is an NK cell or a cytotoxic T cell, Component 3 can part or all of NKG2D, CD352, NKp46, or CD16a. Similarly, where the effector cell is a CD8+ T cell, part or all of 4-1BB, OX40, GITR, CD28, CD27, or ICOS can be Component 3. In some embodiments, Component 3 comprises part of CD3ε. For example, Component 3 may comprise the first 27 amino acids of CD3ε, which may be a mature human CD3ε (SEQ ID NO:50) or a CD3ε from different species, particularly one of the mammalian species listed above such as cynomolgus monkey (SEQ ID NO:51).

In some embodiments, Component 3 can comprise a peptide selected in vitro, which, when it is part of a PABP, can block or inhibit the binding of a PABP to an effector cell or a target cell as compared to binding observed with the same PABP when protease cleavage has separated Component 3 from the remainder of the PABP. Alternatively or in addition, a Component 3 comprising such an in vitro-selected peptide may, when it is part of a PAPB, inhibit cytolysis of target cells in the presence of effector cells and the PABP as compared to the cytolysis observed in the presence of the same effector cells and PABP when protease cleavage has separated the Component 3 from the remainder of the PABP.

### Component 4

Component 4 comprises a protease cleavage site. The cleavage site can be cleaved by a protease that is specifically expressed in the physical vicinity of pathogens, cells infected by pathogens, or cells that mediate a disease, for example, cancer cells. The protease can, for example, be a metalloproteinase, a matrix metalloproteinase (MMP) such as MMP2, MMP9, or MMP11, a serine protease, a cysteine protease, a furin, a plasmin, or a plasminogen activator (such as urokinase-type plasminogen activator (u-PA) or tissue plasminogen activator (tPA)), fibroblast activation protein a (FAP α ), among many others.

These protease cleavage sites can include, for example, sites cleaved by plasmin. The pro-enzyme plasminogen is activated by proteolytic cleavage by u-PA leading to its conversion to the active enzyme, plasmin. Plasmin, a serine protease, may play a role in metastasis due to its degradation of extracellular matrix and its activation of other enzymes, for example, type-IV collagenase. See, e.g., Kaneko et al. (2003), Cancer Sci. 94(1): 43-39, the relevant portions of which are incorporated herein by reference.

Such protease cleavage sites also include, for example, cleavage sites for the metalloproteases meprin a and meprin β, which may be involved in diseases such as certain cancers, inflammatory bowel diseases, cystic fibrosis, kidney diseases, diabetic nephropathy, and dermal fibrotic tumors. The cleavage sites of meprins a and β are not limited to a single, defined sequence for each of these proteases. However, at certain amino acid positions relative to the cleavage site, there is a strong preference for one or a handful of specific amino acids. See, e.g., Becker-Pauly et al. (2011), Molecular and Cellular Proteomics 10(9):M111.009233. D01:10.1074/mcp.M111.009233, the portions of which describe particular cleavage site, including the supplementary material, are incorporated herein by reference. A small selection of known cleavage sites for various proteases, including meprin a and meprin β, are provided in Table 2 below. Component 4 of the invention described herein can contain a cleavage site for any metalloprotease, including meprin a and meprin β, and including, without limitation, any of the cleavage sites listed in Table 2.

Similarly, the matrix metalloproteinases (MMPs) MMP-2 and MMP-9 are overexpressed in a variety of human tumors, including ovarian, breast, and prostate tumors, as well as in melanoma. Moreover, an association between aggressive tumor growth and high levels of MMP-2 and/or MMP-9 has been observed in both clinical and experimental studies. See, e.g., Roomi et al. (2009), Onc. Rep. 21: 1323-1333. An MMP-2 or MMP-9 cleavage site can be represented as P4-P3-P2-P1|P1′-P2′-P3′-P4′, where P1-P4 and P1′-P4′ are amino acids and the vertical line represents the cleavage site. Some generalizations can be made about an MMP-2 cleavage site. P1 is most likely to be glycine or proline. P2 is most likely to be proline, with alanine, valine, or isoleucine being somewhat less likely. P3 is mostly likely to be alanine, serine, or arginine. P4 is most likely to be alanine, glycine, asparagine, or serine. P1′ is most likely to be leucine, with isoleucine, phenylalanine, or tyrosine being somewhat less likely. P2′ is most likely to be lysine, with alanine, valine, isoleucine, or tyrosine being somewhat less likely. P3′ is most likely to be alanine, serine, or glycine. P4′ is most likely to be alanine, lysine, or aspartic acid. There are somewhat clearer preferences for MMP-9 cleavage sites. P4 is most likely to be glycine. P3 is most likely proline. P2 is most likely to be lysine. P1 is most likely to be glycine or proline. P1 is most likely to be leucine, with isoleucine being somewhat less likely. P2′ is most likely to be lysine . P3′ is most likely to be glycine or alanine. P4′ is most likely to alanine, proline, or tyrosine. Any MMP-2 or MMP-9 cleavage site can be contained in Component 4 of the invention described herein, including those disclosed in Table 2 or in, e.g., Prudova et al. (2010), Mol. Cell. Proteomics 9(5): 894-911, the relevant portions of which are incorporated herein by reference.

Higher-than-normal levels of u-PA are known to be associated with various cancers, including, for example colorectal cancer, breast cancer, monocytic and myelogenous leukemias, bladder cancer, thyroid cancer, liver cancer, gastric cancer, and cancers of the pleura, lung, pancreas, ovaries, and the head and neck. See, e.g., Skelly et al. (1997), Clin. Can. Res. 3: 1837-1840; Han et al. (2005), Oncol. Rep. 14(1): 105-112; Kaneko et al. (2003), Cancer Sci. 94(1): 43-49; Liu et al. (2001), J. Biol. Chem. 276(21): 17976-17984. In Table 2 below a small sample of sites that can be cleaved by u-PA are reported. Component 4 of the invention described herein can contain a cleavage site for any serine protease, including u-PA and tissue plasminogen activator (tPA), and including any of the cleavage sites listed in Table 2.

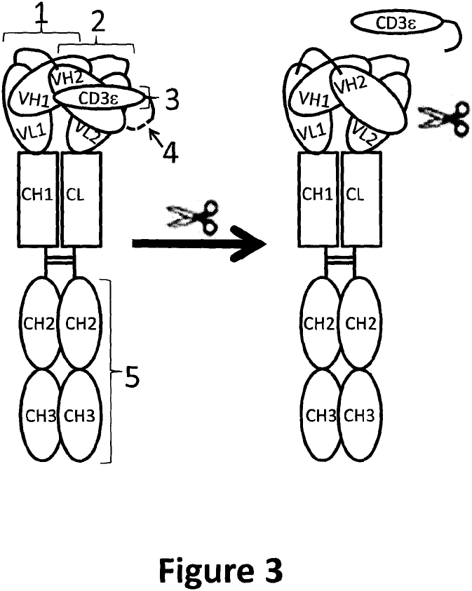

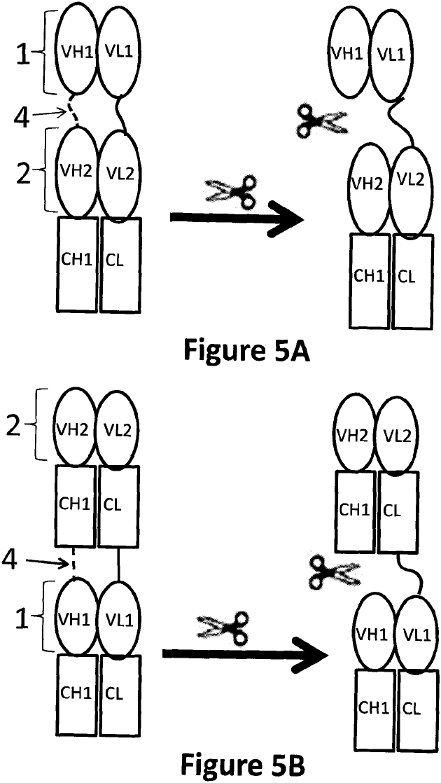

Some cysteine proteases, such as cathepsin B, have been found to be overexpressed in tumor tissue and likely play a causative role in some cancers. See, e.g., Emmert-Buck et al. (1994), Am. J. Pathol. 145(6): 1285-1290; Biniosseek et al. (2011), J. Proteome Res. 10: 5363-5373. The portions of these references that describe protease cleavage sites are incorporate herein by reference. As with cleavage sites for meprin a and meprin β, there is a lot of heterogeneity in cathepsin B cleavage sites. A cleavage site for cathepsin B (as well as other proteases) can be represented as P3-P2-P1IP1′-P2′-P3′, where P1-P3 and P1′-P3′ are all amino acids and vertical line represents the cleavage site. Some generalizations apply to cathepsin B cleavage sites. P3 is most often G, F, L, or P (using one letter code for amino acids). P2 is most often A, V, Y, F, or I. P1 is most often G, A, M, Q, or T. P1′ is most often F, G, I, V, or L. P2′ is most often V, I, G, T, or A. P3′ is most often G. Further there is some subsite cooperatively. For example, if P2 is F, then P3 is most likely to be G and least likely to be L, and P1′ is most likely to be F and least likely to be L. This and other examples of subsite cooperativity are described in detail in Biniossek et al. (2011), J. Proteome Res. 10: 5363-5373. FIGS. 3 and 5 of Biniossek, and accompanying text, plus Supplementary Table 1 are incorporated herein by reference. All cathepsin B cleavage sites, including without limitation those in Table 2, can be contained in Component 4 of the invention described herein.

TABLE 2

Examples of Protease Cleavage Sites

| | |
|---|---|
| Protease | Sequence of cleavage site* |
| meprin a meprin β | APMAIEGGG (SEQ ID NO:55) |
| | EAQG\|DK\|\| (SEQ ID NO:56) |
| | LAFSIDAGP (SEQ ID NO:57) |
| | YVA\|DAPK (SEQ ID NO:58) |
| u-PA | SGRISA (SEQ ID NO:59) |
| | GSGRISA (SEQ ID NO:60) |
| | SGK\|SA (SEQ ID NO:61) |
| u-PA | SGRISS (SEQ ID NO:62) |
| | SGRIRA (SEQ ID NO:63) |
| | SGRINA (SEQ ID NO:64) |
| | SGRIKA (SEQ ID NO:65) |
| tPA | QRGRISA (SEQ ID NO:66) |
| cathepsin B | TQGIAAA (SEQ ID NO:67) |
| | GAAIAAA (SEQ ID NO:68) |
| | GAGIAAG (SEQ ID NO:69) |
| | AAAIAAG (SEQ ID NO:70) |
| | LCGIAAI (SEQ ID NO:71) |
| | FAQIALG (SEQ ID NO:72) |
| | LAAIANP (SEQ ID NO:73) |
| | LLQIANP (SEQ ID NO:74) |
| | LAAIANP (SEQ ID NO:75) |
| | LYG\|AQF (SEQ ID NO:76) |
| | LSQIAQG (SEQ ID NO:77) |
| | ASAIASG (SEQ ID NO:78) |
| | FLGIASL (SEQ ID NO:79) |
| | AYG\|ATG (SEQ ID NO:80) |
| | LAQIATG (SEQ ID NO:81) |
| MMP-2 | GPLGIIAGQ (SEQ ID NO:1) |
| | GGPLGIMLSQS (SEQ ID NO:2) |
| | PLGILAG (SEQ ID NO:3) |
| MMP-11 | AANILRN (SEQ ID NO:95) |
| | AQA\|YVK (SEQ ID NO:96) |
| | AAN\|YMR (SEQ ID NO:97) |
| | AAAILTR (SEQ ID NO:98) |
| | AQNILMR (SEQ ID NO:99) |
| | AAN\|YTK (SEQ ID NO:100) |
| Furin | RRRRR (SEQ ID NO:4) |
| | RRRRRR (SEQ ID NO:82) |
| | GQSSRHRRAL (SEQ ID NO:5) |

*vertical lines represent the predicted cleavage site

Component 4 and other portions of a PABP can contain “linker” sequences that are not protease cleavable. For example, Component 4 can contain a protease cleavage site and other linker sequences that are not cleavable. Alternatively, Component 4 may contain only a protease cleavage site. These non-cleavable linkers can include amino acid sequences such as, for example (G₄S)ₙ, where n can be, for example, 1, 2, 3, 4, 5, 6, 7, or 8. G₄S is listed as SEQ ID NO:88. Other exemplary linkers include, for example, the amino acid sequences TVAAP (SEQ ID NO:89), ASTKGP (SEQ ID NO:90), GGGGSAAA (SEQ ID NO:91), GGGGSGGGGSGGGGS (SEQ ID NO:92), and AAA, among many others.

### Component 5

A half life-extending moiety can be, for example, an Fc polypeptide, albumin, an albumin fragment, a moiety that binds to albumin or to the neonatal Fc receptor (FcRn), a derivative of fibronectin that has been engineered to bind albumin or a fragment thereof, a peptide, a single domain protein fragment, or other polypeptide that can increase serum half life. In alternate embodiments, a half life-extending moiety can be a non-polypeptide molecule such as, for example, polyethylene glycol (PEG). Sequences of human IgG1, IgG2, IgG3, and IgG4 Fc polypeptides that could be used are provided in SEQ ID NOs:84-87. Variants of these sequences containing one or more heterodimerizing alterations, one or more Fc alteration that extends half life, one or more alteration that enhances ADCC, and/or one or more alteration that inhibits Fc gamma receptor (FcyR) binding are also contemplated, as are other close variants containing not more than 10 deletions, insertions, or substitutions of a single amino acid per 100 amino acids of sequence.

The sequence of a derivative of human fibronectin type III (Fn3) engineered to bind albumin is provided in SEQ ID NO:83. As is known in the art, the loops of a human fibronectin type III (Fn3) domain can be engineered to bind to other targets. Koide (1998), J Mol Biol.: 284(4): 1141-51.

The half life extending moiety can be an Fc region of an antibody. If so, the first polypeptide chain can contain an Fc polypeptide chain after the CH1 region, and the second polypeptide chain can contain an Fc polypeptide chain after the CL region. Alternatively, only one polypeptide chain can contain an Fc polypeptide chain. There can be, but need not be, a linker between the CH1 region and the Fc region and/or between the CL region and the Fc region. As explained above, an Fc polypeptide chain comprises all or part of a hinge region followed by a CH2 and a CH3 region. The Fc polypeptide chain can be of mammalian (for example, human, mouse, rat, rabbit, dromedary, or new or old world monkey), avian, or shark origin. In addition, as explained above, an Fc polypeptide chain can include a limited number alterations. For example, an Fc polypeptide chain can comprise one or more heterodimerizing alterations, one or more alteration that inhibits or enhances binding to FcyR, or one or more alterations that increase binding to FcRn.

In some embodiments the amino acid sequences of the Fc polypeptides can be mammalian, for example a human, amino acid sequences. The isotype of the Fc polypeptide can be IgG, such as IgG1, IgG2, IgG3, or IgG4, IgA, IgD, IgE, or IgM. Table 2 below shows an alignment of the amino acid sequences of human IgG1, IgG2, IgG3, and IgG4 Fc polypeptide chains.

TABLE 2

Amino acid sequences of human IgG Fc polypeptide chains
IgG1 -----------------------------------------------
IgG2 -----------------------------------------------
IgG3 ELKTPLGDTTHTCPRCPEPKSCDTPPPCPRCPEPKSCDTPPPCPRCP
IgG4 -----------------------------------------------
225 235 245 255 265 275
* * * * * *
IgG1 EPKSCDKTHTCPPCPAPELLGGPSVFLFPPKPKDTLMISRTPEVTCVVVDVSHEDPEVKF
IgG2 ERKCCVE---CPPCPAPPVA-GPSVFLFPPKPKDTLMISRTPEVTCVVVDVSHEDPEVQF
IgG3 EPKSCDTPPPCPRCPAPELLGGPSVFLFPPKPKDTLMISRTPEVTCVVVDVSHEDPEVQF
IgG4 ESKYG---PPCPSCPAPEFLGGPSVFLFPPKPKDTLMISRTPEVTCVVVDVSQEDPEVQF
285 295 305 315 325 335
* * * * * *
IgG1 NWYVDGVEVHNAKTKPREEQYNSTYRVVSVLTVLHQDWLNGKEYKCKVSNKALPAPIEKT
IgG2 NWYVDGMEVHNAKTKPREEQFNSTFRVVSVLTVVHQDWLNGKEYKCKVSNKGLPAPIEKT
IgG3 KWYVDGVEVHNAKTKPREEQYNSTFRVVSVLTVLHQDWLNGKEYKCKVSNKALPAPIEKT
IgG4 NWYVDGVEVHNAKTKPREEQFNSTYRVVSVLTVLHQDWLNGKEYKCKVSNKGLPSSIEKT
345 355 365 375 385 395
* * * * * *
IgG1 ISKAKGQPREPQVYTLPPSREEMTKNQVSLTCLVKGFYPSDIAVEWESNGQPENNYKTTP
IgG2 ISKTKGQPREPQVYTLPPSREEMTKNQVSLTCLVKGFYPSDIAVEWESNGQPENNYKTTP
IgG3 ISKTKGQPREPQVYTLPPSREEMTKNQVSLTCLVKGFYPSDIAVEWESSGQPENNYNTTP
IgG4 ISKAKGQPREPQVYTLPPSQEEMTKNQVSLTCLVKGFYPSDIAVEWESNGQPENNYKTTP
405 415 425 435 445
* * * * *
IgG1 PVLDSDGSFFLYSKLTVDKSRWQQGNVFSCSVMHEALHNHYTQKSLSLSPGK (SEQ ID NO:84)
IgG2 PMLDSDGSFFLYSKLTVDKSRWQQGNVFSCSVMHEALHNHYTQKSLSLSPGK (SEQ ID NO:85)
IgG3 PMLDSDGSFFLYSKLTVDKSRWQQGNIFSCSVMHEALHNRFTQKSLSLSPGK (SEQ ID NO:86)
IgG4 PVLDSDGSFFLYSRLTVDKSRWQEGNVFSCSVMHEALHNHYTQKSLSLSLGK (SEQ ID NO:87)

The numbering shown in Table 2 is according the EU system of numbering, which is based on the sequential numbering of the constant region of an IgG1 antibody. Edelman et al. (1969), Proc. Natl. Acad. Sci. 63: 78-85. Thus, it does not accommodate the additional length of the IgG3 hinge well. It is nonetheless used here to designate positions in an Fc region because it is still commonly used in the art to refer to positions in Fc regions. The hinge regions of the IgG1, IgG2, and IgG4 Fc polypeptides extend from about position 216 to about 230. It is clear from the alignment that the IgG2 and IgG4 hinge regions are each three amino acids shorter than the IgG1 hinge. The IgG3 hinge is much longer, extending for an additional 47 amino acids upstream. The CH2 region extends from about position 231 to 340, and the CH3 region extends from about position 341 to 447.

Naturally occurring amino acid sequences of Fc polypeptides can be varied slightly. Such variations can include no more that 10 insertions, deletions, or substitutions of a single amino acid per 100 amino acids of sequence of a naturally occurring Fc polypeptide chain. If there are substitutions, they can be conservative amino acid substitutions, as defined above. The Fc polypeptides on the first and second polypeptide chains can differ in amino acid sequence. In some embodiments, they can include “heterodimerizing alterations,” for example, charge pair substitutions, as defined above, that facilitate heterodimer formation. Further, the Fc polypeptide portions of the PABP can also contain alterations that inhibit or enhance FcyR binding. Such mutations are described above and in Xu et al. (2000), Cell Immunol. 200(1): 16-26, the relevant portions of which are incorporated herein by reference. The Fc polypeptide portions can also include an “Fc alteration that extends half life,” as described above, including those described in, e.g., U.S. Pat. 7,037,784, 7,670,600, and 7,371,827, U.S. Pat. Application Publication 2010/0234575, and International Application PCT/US2012/070146, the relevant portions of all of which are incorporated herein by reference. Further, an Fc polypeptide can comprise “alterations that enhance ADCC,” as defined above.

### Various Embodiments of a Protease-Activatable Bispecific Molecule

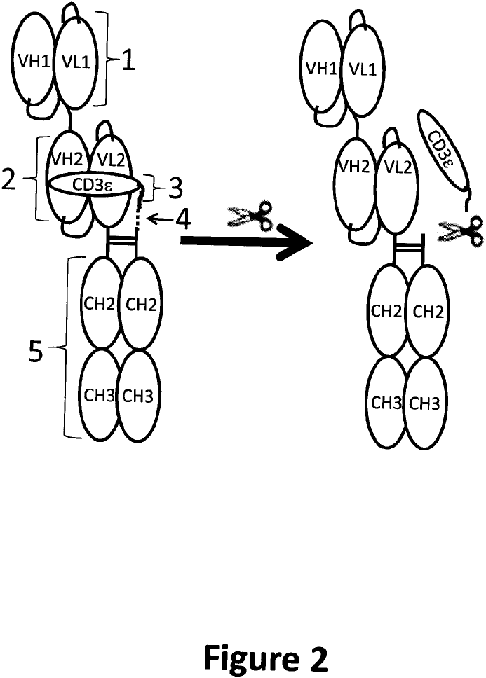

FIG. 2 is a diagram of an example of a PABP, as described herein. The ovals labeled “VH1” and “VL1” represent heavy and light chain variable (VH and VL) regions that, together, can bind to a target molecule expressed on a disease-mediating cell, for example, a cancer cell antigen, or on an infected cell or a pathogen. As indicated, VH1 and VL1, together, comprise Component 1 as discussed above in connection with FIG. 1. As indicated, the ovals labeled “VH2” and “VL2” represent VH and VL regions that, together, can bind to CD3ε and comprise Component 2. The smaller oval labeled “CD3ε” represents a portion of CD3ε to which VH2 and VL2 bind and, hence, comprises Component 3 as discussed above. As discussed in connection with Components 2 and 3, Component 3 could be a protein other than CD3ε that is expressed on a T cell, an NK cell, a monocyte, a macrophage, or a neutrophil. The dashed line indicated by a “4” and an arrow represents a protease cleavage site (corresponding to Component 4 discussed above). Other curving lines represent non-cleavable linkers. The straight lines extending upwards from the CH2 regions, which are joined by horizontal lines, are disulfide-bonded hinge regions. The ovals labeled “CH2” and “CH3,” along with part or all of a hinge region, represent an Fc polypeptide chain, which can prolong half life. As indicated, the Fc region is considered to be Component 5.

Another embodiment is diagrammed in FIG. 3. As indicated, one polypeptide chain comprises a fragment of CD3ε (Component 3), followed by VH2, a linker, VL1, CH1 and an Fc polypeptide chain. The other polypeptide chain comprises VH1, followed by a linker, VL2, CL, and an Fc polypeptide chain. VH2 and VL2 can bind to CD3ε. As indicated, the dashed curving line represents a protease cleavage site (Component 4), and straight and curving lines represent hinges regions and linkers, as indicated above.

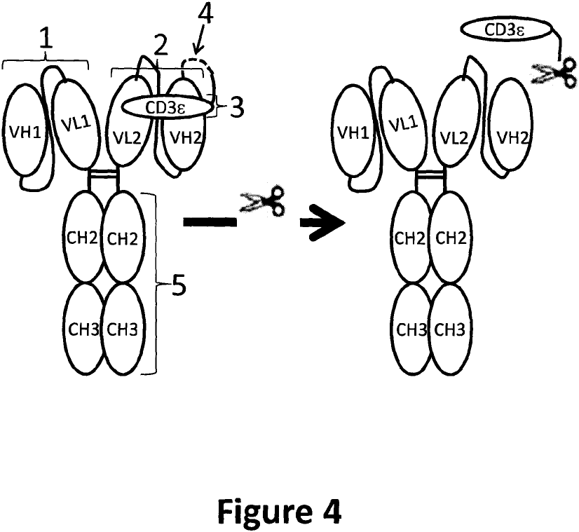

A further embodiment is diagrammed in FIG. 4. One polypeptide chain comprises an scFv comprising VH1 and VL1 (ovals labeled “VH1” and “VL1”), which are from an antibody that binds to a target cell molecule, an optional linker, and an Fc polypeptide chain (hinge and ovals labeled “CH2” and “CH3”). The other polypeptide comprises a portion of CD3ε, which, as indicated, is Component 3 of the PABP. This is followed by an scFv comprising VH2 and VL2, which are from an antibody that binds CD3ε, followed by and optional linker and an Fc polypeptide chain. The dashed line represents a protease cleavage site, i.e., Component 4, as indicated. Curving lines indicate linker sequences. The straight vertical lines extending upward from the CH2 regions joined by horizontal lines represent hinge regions joined by disulfide bridges. As explained in connection with FIG. 2, Component 3 could be a protein other than CD3ε, and VL2 and VH2 could bind to it.

Still other embodiments are shown in FIGS. 5A and 5B. FIG. 5A represents a protein where one polypeptide comprises a VH1 followed by a protease cleavage site (Component 4), followed by VH2 and a CH1. The other polypeptide comprises a VL1 followed by a linker, a VL2, and a CL. As indicated, VH1 and VL1 represent Component 1, and VH2 andVL2 represent Component 2.

FIG. 5B represents a protein comprising a polypeptide including a VH2 followed by a CH1, a protease cleavage site (Component 4), VH1, and CH1. The other polypeptide comprises a VL2, CL, a linker, a VL1, and CL. As indicated, VH1 and VL1 represent Component 1, and VH2 and VL2 represent Component 2.

### Nucleic Acids Encoding PABPs

Provided are nucleic acids encoding the PABPs described herein. Numerous nucleic acid sequences encoding immunoglobulin regions including VH, VL, hinge, CH1, CH2, CH3, and CH4 regions are known in the art. See, e.g., Kabat et al. in SEQUENCES OF IMMUNOLOGICAL INTEREST, Public Health Service N.I.H., Bethesda, MD, 1991. Using the guidance provided herein, one of skill in the art could combine such nucleic acid sequences and/or other nucleic acid sequence known in the art to create nucleic acid sequences encoding the PABPs described herein.

In addition, nucleic acid sequences encoding PABPs described herein can be determined by one of skill in the art based on the amino acid sequences provided herein and knowledge in the art. Besides more traditional methods of producing cloned DNA segments encoding a particular amino acid sequence, companies such as DNA 2.0 (Menlo Park, CA, USA) and BlueHeron (Bothell, WA, USA), among others, now routinely produce chemically synthesized, gene-sized DNAs of any desired sequence to order, thus streamlining the process of producing such DNAs.

### Methods of Making the PABPs

The PABPs described herein can be made using methods well known in the art. For example, nucleic acids encoding the two polypeptide chains of a PABP can be introduced into a cultured host cell by a variety of known methods, such as, for example, transformation, transfection, electroporation, bombardment with nucleic acid-coated microprojectiles, etc. In some embodiments the nucleic acids encoding the PABPs can be inserted into a vector appropriate for expression in the host cells before being introduced into the host cells. Typically such vectors can contain sequence elements enabling expression of the inserted nucleic acids at the RNA and protein levels. Such vectors are well known in the art, and many are commercially available. The host cells containing the nucleic acids can be cultured under conditions so as to enable the cells to express the nucleic acids, and the resulting PABPs can be collected from the cell mass or the culture medium. Alternatively, the PABPs can be produced in vivo, for example in plant leaves (see, e.g., Scheller et al. (2001), Nature Biotechnol. 19: 573-577 and references cited therein), bird eggs (see, e.g., Zhu et al. (2005), Nature Biotechnol. 23: 1159-1169 and references cited therein), or mammalian milk (see, e.g., Laible et al. (2012), Reprod. Fertil. Dev. 25(1): 315).

A variety of cultured host cells can be used including, for example, bacterial cells such as Escherichia coli or Bacilis steorothermophilus, fungal cells such as Saccharomyces cerevisiae or Pichia pastoris, insect cells such as lepidopteran insect cells including Spodoptera frugiperda cells, or mammalian cells such as Chinese hamster ovary (CHO) cells, baby hamster kidney (BHK) cells, monkey kidney cells, HeLa cells, human hepatocellular carcinoma cells, or 293 cells, among many others.

### Therapeutic Methods and Compositions

The PABPs described herein can be used to treat a wide variety of conditions including, for example, various forms of cancer, infections, fibrotic diseases, and/or autoimmune or inflammatory conditions.

Provided herein are pharmaceutical compositions comprising the PABPs described herein. Such pharmaceutical compositions comprise a therapeutically effective amount of a PABP, as described herein, plus one or more additional components such as a physiologically acceptable carrier, excipient, or diluent. Such additional components can include buffers, carbohydrates, polyols, amino acids, chelating agents, stabilizers, and/or preservatives, among many possibilities.

In some embodiments, the PABPs described herein can be used to treat cell proliferative diseases, including cancer, which involve the unregulated and/or inappropriate proliferation of cells, sometimes accompanied by destruction of adjacent tissue and growth of new blood vessels, which can allow invasion of cancer cells into new areas, i.e., metastasis. These conditions include hematologic malignancies and solid tumor malignancies. Included within conditions treatable with the PABPs described herein are non-malignant conditions that involve inappropriate cell growth, including colorectal polyps, cerebral ischemia, gross cystic disease, polycystic kidney disease, benign prostatic hyperplasia, and endometriosis. Other cell proliferative diseases that can be treated using the PABPs of the present invention are, for example, cancers including mesotheliomas, squamous cell carcinomas, myelomas, osteosarcomas, glioblastomas, gliomas, carcinomas, adenocarcinomas, melanomas, sarcomas, acute and chronic leukemias, lymphomas, and meningiomas, Hodgkin’s disease, Sézary syndrome, multiple myeloma, and lung, non-small cell lung, small cell lung, laryngeal, breast, head and neck, bladder, ovarian, skin, prostate, cervical, vaginal, gastric, renal cell, kidney, pancreatic, colorectal, endometrial, and esophageal, hepatobiliary, bone, skin, and hematologic cancers, as well as cancers of the nasal cavity and paranasal sinuses, the nasopharynx, the oral cavity, the oropharynx, the larynx, the hypolarynx, the salivary glands, the mediastinum, the stomach, the small intestine, the colon, the rectum and anal region, the ureter, the urethra, the penis, the testis, the vulva, the endocrine system, the central nervous system, and plasma cells.

Among the texts providing guidance for cancer therapy is Cancer, Principles and Practice of Oncology, 4th Edition, DeVita et al., Eds. J. B. Lippincott Co., Philadelphia, PA (1993). An appropriate therapeutic approach is chosen according to the particular type of cancer, and other factors such as the general condition of the patient, as is recognized in the pertinent field. The PABPs described herein may be added to a therapy regimen using other anti-neoplastic agents and/or treatments in treating a cancer patient.

In some embodiments, the PABPs can be administered concurrently with, before, or after a variety of drugs and treatments widely employed in cancer treatment such as, for example, chemotherapeutic agents, non-chemotherapeutic, anti-neoplastic agents, and/or radiation. For example, chemotherapy and/or radiation can occur before, during, and/or after any of the treatments described herein. Examples of chemotherapeutic agents are discussed above and include, but are not limited to, cisplatin, taxol, etoposide, mitoxantrone (Novantrone®), actinomycin D, cycloheximide, camptothecin (or water soluble derivatives thereof), methotrexate, mitomycin (e.g., mitomycin C), dacarbazine (DTIC), anti-neoplastic antibiotics such as adriamycin (doxorubicin) and daunomycin, and all the chemotherapeutic agents mentioned above.

The PABPs described herein can also be used to treat infectious disease, for example a chronic hepatis B virus (HBV) infection, a hepatis C virus (HPC) infection, a human immunodeficiency virus (HIV) infection, an Epstein-Barr virus (EBV) infection, or a cytomegalovirus (CMV) infection, among many others.

The PABPs described herein can find further use in other kinds of conditions where it is beneficial to deplete certain cell types. For example, depletion of human eosinophils in asthma, excess human B cells in systemic lupus erythematosus, excess human Th2 T cells in autoimmune conditions, or pathogen-infected cells in infectious diseases can be beneficial. Depletion of myofibroblasts or other pathological cells in fibrotic conditions such as lung fibrosis, such as idiopathic pulmonary fibrosis (IPF), or kidney or liver fibrosis is a further use of a PABP.

Therapeutically effective doses of the PABPs described herein can be administered. The amount of antibody that constitutes a therapeutically dose may vary with the indication treated, the weight of the patient, the calculated skin surface area of the patient. Dosing of the PABPs described herein can be adjusted to achieve the desired effects. In many cases, repeated dosing may be required. For example, a PABP as described herein can be dosed three times per week, twice per week, once per week, once every two, three, four, five, six, seven, eight, nine, or ten weeks, or once every two, three, four, five, or six months. The amount of the PABP administered on each day can be from about 0.0036 mg to about 450 mg. Alternatively, the dose can calibrated according to the estimated skin surface of a patient, and each dose can be from about 0.002 mg/m² to about 250 mg/m². In another alternative, the dose can be calibrated according to a patient’s weight, and each dose can be from about 0.000051 mg/kg to about 6.4 mg/kg.

The PABPs, or pharmaceutical compositions containing these molecules, can be administered by any feasible method. Protein therapeutics will ordinarily be administered by a parenteral route, for example by injection, since oral administration, in the absence of some special formulation or circumstance, would lead to hydrolysis of the protein in the acid environment of the stomach. Subcutaneous, intramuscular, intravenous, intraarterial, intralesional, or peritoneal injection are possible routes of administration. A PABP can also be administered via infusion, for example intravenous or subcutaneous infusion. Topical administration is also possible, especially for diseases involving the skin. Alternatively, a PABP can be administered through contact with a mucus membrane, for example by intra-nasal, sublingual, vaginal, or rectal administration or administration as an inhalant. Alternatively, certain appropriate pharmaceutical compositions comprising a PABP can be administered orally.

Having described the invention in general terms above, the following examples are offered by way of illustration and not limitation.

### EXAMPLES

### Example 1: Construction and Production of PABPs and Control Proteins

PABPs were made by introducing DNA encoding amino acids 1-27 of mature human CD3ε plus a linker, i.e., (G₄S)₃, and/or a protease cleavage site into pre-existing DNA constructs. For example, in the cases of CD3ε(1-27)-aCD3-aHER2-Xbody, CD3ε(1-27)-MMP-2csV1-aCD3-aHER2-Xbody, CD3ε(1-27)-FURINcsV1-aCD3-aHER2-Xbody, CD3ε(1-27)-MMP-2csV2-aCD3-aHER2-Xbody, CD3ε(1-27)-FURINcsV2-aCD3-aHER2-Xbody, CD3ε(1-27)-MMP-2csV3-aCD3-aHER2-Xbody, the pre-existing DNA construct encoded a bispecific protein (called aCD3-aHER2-Xbody) comprising the amino acid sequences of SEQ ID NOs:6 and 93, which is described in International Application PCT/US/2014/026658, the relevant portions of which are incorporated herein by reference. The inserts comprising the CD3ε fragment and the linkers and/or protease cleavage sites were introduced by PCR using appropriate primers and the constructs were finished by Gibson assembly as explained in Gibson et al. (2009), Nature Methods 6(5): 343-343. The portions of this reference explaining how this method is performed are incorporated herein by reference. Briefly, double-stranded DNA fragments having overlapping sequences on the ends were incubated with T5 exonuclease (which recess double-stranded DNA from 5′ ends), PHUSION® DNA polymerase (New England Biolabs), and Taq ligase at 50° C. and subsequently used to transform Escherichacoli to obtain colonies containing DNA constructs having the desired sequences.

DNA constructs encoding the PABPs CD3ε(1-27)-aCD3-aHER2-mxb, CD3ε(1-27)-MMP-2csV1-aCD3-aHER2-mxb, CD3ε(1-27)-MMP-2csV2-aCD3-aHER2-mxb, and CD3ε(1-27)-FURINcsV2-aCD3-aHER2-mxb were constructed in a similar way starting with a DNA construct encoding aCD3-aHER2-mxb, which comprises the amino acid sequences of SEQ ID NOs:20 and 94.

Similarly, DNA constructs encoding CD3ε(1-27)-aCD3-aHER2-BiFc, CD3ε(1-27)-MMP-cs-aCD3-aHER2-BiFc, and CD3ε(1-27)-FURINcs-aCD3-aHER2-BiFc were made starting with a DNA construct encoding aCD3-aHER2-Bi-Fc, which comprises the amino acid sequences of SEQ ID NOs:30 and 32.

The proteins were produced by transient transfection into HEK 293-6e cells, and protein was purified from the conditioned media.

### Example 2: MMP Cleavage Sites Can Be Digested in Vitro

To assess cleavage of various PABPs by MMP-2, the proteins to be assayed were diluted to 100 ng/µl in phosphate buffered saline (PBS) with 30 µM ZnCl₂. MMP-2 protease (Calbiochem (Cat#PF023)) was added (0.5 µl at 0.1 mg/ml) to 20 µl (containing 2000 ng) of the solution containing the PABP and incubated overnight at 37° C. Thereafter, digested protein from the protease reaction (0.5 ul (50 ng)), plus undigested protein, was loaded onto a NUPAGE® NOVEX® 4-12% Bis-Tris Gel (Life Technologies, Grand Island, New York) and run with MES buffer under reducing conditions. The gel was transferred by western blot, and the bispecific proteins were detected using a horse radish peroxidase (HRP)-conjugated anti-human-Fc antibody.

FIG. 6 shows some of these results for constructs having the general format shown in FIG. 3. The two polypeptide chains of each heterodimeric PABP appear as two bands that are close in size. Antibodies lacking an MMP2 cleavage site, some of which contain furin cleavage sites, do not change in size when digested with MMP2. See lanes 1 and 2, 5 and 6, and 9 and 10. In PABPs containing an MMP2 cleavage site, one of the two polypeptide chains decreases in size upon digestion with MMP2. See lanes 3 and 4, 7 and 8, and 11 and 12. In addition, PABPs containing a furin cleavage site are recovered from conditioned media as fully (CD3ε-FURINcsV2-aCD3-aHER2-Xbody; lanes 9 and 10) or partially (CD3ε-FURINcsV1-aCD3-aHER2-Xbody; lanes 5 and 6) cleaved proteins. As is known in the art, HEK-293 cells express furin protease intracellularly, which has been observed to cleave recombinant proteins produced in HEK-293 cells. See, e.g., Wu et al. (2003), J. Biol. Chem. 278: 25847-25852. Presumably, these intracellular furins are responsible for the cleavage of PABPs containing furin cleavage sites.

Similar experiments were performed to determine whether an MMP2 cleavage sites in bispecific scFv-Fc PABPs having the general format shown in FIG. 4 could be cleaved in vitro. Digestions with MMP2 and gel electrophoresis were performed as described above. Most of the antibodies, with the exception of the one containing a furin site (CD3ε-FURINcsV2-aCD3-aHER2-mxb; FIG. 7, lanes 7 and 8), appear as two distinct bands close in size. CD3ε-FURINcsV2-aCD3-aHER2-mxb appears as a single band, indicating that the furin cleavage site has been cleaved. PABPs that did not contain an MMP2 cleavage site did not change in size upon digestion with MMP2. See FIG. 7, lanes 1 and 2 and lanes 7 and 8. In antibodies that did contain an MMP2 site, the upper band became weaker with MMP2 digestion, and the lower band became more intense relative to the upper band, suggesting that the MMP2 cleavage site was partially cleaved. See FIG. 7, lanes 3 and 4 and lanes 5 and 6.

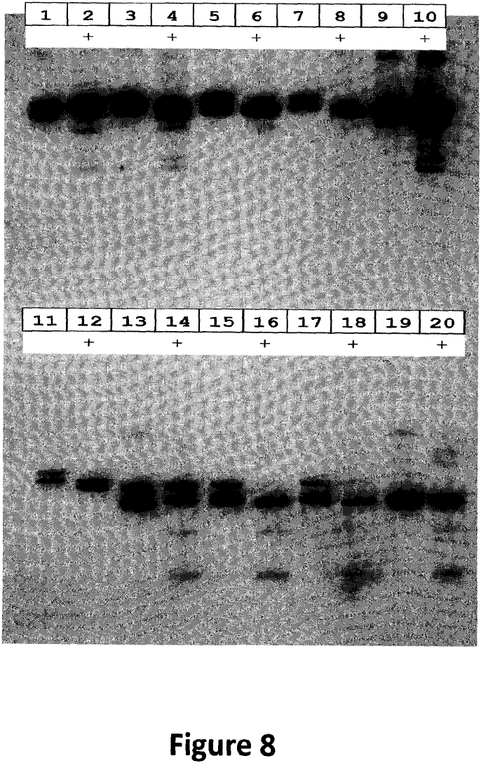

Using the PABPs described above, an additional experiment was done to determine whether the MMP2 cleavage sites in these PABPs could be cleaved by MMP9 in vitro. PABPs containing an MMP2 cleavage site were clipped by digestion with MMP9. See FIG. 8, lanes 3 and 4, 7 and 8, 11 and 12, 15 and 16, and 17 and 18. In addition, a PABP containing a furin cleavage site appeared to be at least partially cleaved by MMP9 (FIG. 8, lanes 5 and 6), and a number of MMP9 digestions produced smaller bands (FIG. 8, lanes 2, 4, 10, 14, 16, 18, and 20). These data suggest that MMP9 may be less selective than MMP2.

### Example 3: Cytolytic Activity of and T Cell Activation by Heterodimeric Bispecific PABPs

The following experiments tested the in vitro cytolytic activity (T cell-dependent cell cytolysis (TDCC)) of protease digested and undigested PABPs having the general format diagrammed in FIG. 3 and their ability to activate T cells (measured as expression of CD25).

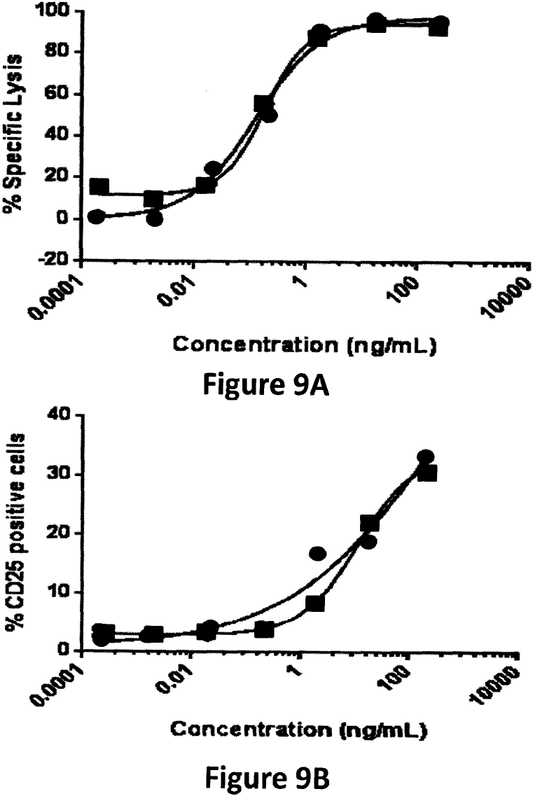

TDCC assays used tumor cells expressing HER2 as target cells, specifically SKOV-3 cells (FIGS. 9A, 10A, 11A, and 12A). SKOV-3 cell express about 530,000 molecules of HER2 protein per cell. Briefly, pan T cells were isolated from healthy human donors using the Pan T Cell Isolation Kit II, human (Miltenyi Biotec, Auburn, CA). The T cells were incubated with carboxyfluorescein succinimidyl ester (CFSE)-labeled tumor target cells at a ratio of 10:1 in the presence or absence of the PABPs at the varying concentrations as indicated in FIGS. 9A, 10A, 11A, and 12A. As a negative control, some samples contained T cells and tumor target cells, but no bispecific protein.

After 40 hours of incubation, cells were harvested, and the percent of tumor cell lysis was monitored by uptake of 7-amino-actinomycin D (7-AAD), which stains double-stranded nucleic acids. Intact cells exclude 7-AAD, whereas 7-AAD can penetrate the membranes of dead or dying cells and stain the double-stranded nucleic acids inside these cells. Percent specific lysis was calculated according to the following formula:

$$\% s p e c i f i c l y s i s = 1 - \frac{l i v e c e l l c o u n t \left(w i t h b i s p e c i f i c\right)}{l i v e c e l l c o u n t s \left(w i t h o u t b i s p e c i f i c\right)} \times 100$$

T cell activation was assessed on the basis of expression of CD25 by the T cells. Pan T cells were isolated from healthy human donors using the Pan T Cell Isolation Kit II, human (Miltenyi Biotec, Auburn, CA). These T cells were incubated with the PABPs described above in the presence of HT-29 cells (which are tumor-derived cells that express HER2) at a T cell:tumor cell ratio of 10:1. After 40 hours of incubation, non-adherent cells were removed from the wells. All samples were stained with allophycocyanin (APC)-conjugated anti-CD25 antibody, a marker of T cell activation and analyzed by FACS.

FIGS. 9A and 9B show the results of positive control experiments, which are TDCC and T cell activation assays of aCD3-aHER2-Xbody and aCD3-aHER2-mxb. These molecules have the general structures diagramed in FIGS. 3 and 4, respectively, except that they lack the CD3ε(1-27) peptide (Component 3) and the linker containing a protease cleavage site that links it to the rest of the molecule. They are expected to be active without protease cleavage. Both molecules have potent cytolytic activity against SKOV-3 cells, having Ec50’s in this assay of less than 1 ng/mL. FIG. 9A; see Table 3, below. Further, addition of either molecule increased the proportion of activated T cells in a concentration dependent manner. FIG. 9B.

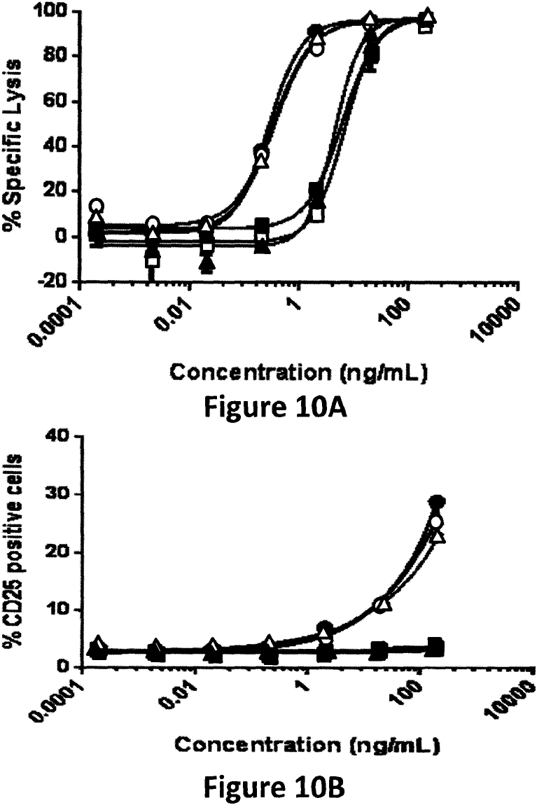

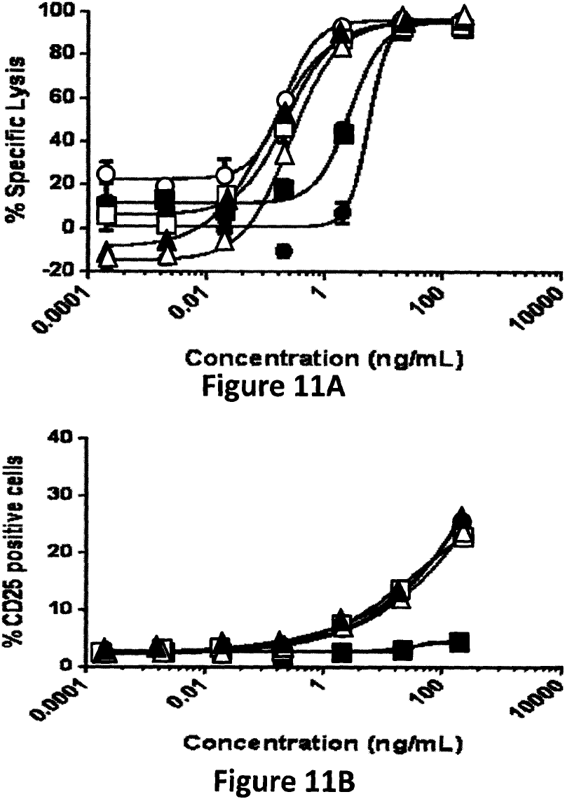

In further samples, anti-CD3ε/HER2 PABPs comprising the CD3ε(1-27) fragment were tested for cytolytic activity and T cell activation with and without digestion by MMP2. In FIGS. 10A, 10B, 11A and 11B, all data is from assays using PABPs having the general structure shown in FIG. 3 and identical amino acid sequences except for the linker connecting the CD3ε fragment to the rest of the molecule. The PABPs are CD3ε(1-27)-MMP-2csV1-aCD3-aHER2-Xbody (linker containing an MMP2 cleavage site), CD3ε(1-27)-FURINcsV1-aCD3-aHER2-Xbody (linker containing an furin cleavage site), CD3ε(1-27)-aCD3-aHER2-Xbody (non-cleavable linker), CD3ε(1-27)-MMP-2csV2-aCD3-aHER2-Xbody (linker containing an MMP2 cleavage site), CD3ε(1-27)-FURINcsV2-aCD3-aHER2-Xbody (linker containing an furin cleavage site), and CD3ε(1-27)-MMP-2csV3-aCD3-aHER2-Xbody (linker containing an MMP2 cleavage site). Since these proteins were made in HEK-293, which produce furin intracellularly, CD3ε(1-27)-FURINcsV1-aCD3-aHER2-Xbody and CD3ε(1-27)-FURINcsV2-aCD3-aHER2-Xbody were likely to be cleaved during production by the HEK-293 cells.

CD3ε27)-FURINcsV1-aCD3-aHER2-Xbody and CD3ε27)-FURINcsV2-aCD3-aHER2-Xbody had an Ec50s of less than 1 ng/mL in the TDCC assay, whether or not they were digested with MMP2. FIGS. 10A and 11A; Table 3. In contrast, CD3ε(1-27)-MMP-2csV1-aCD3-aHER2-Xbody, CD3ε(1-27)-MMP-2csV2-aCD3-aHER2-Xbody, and CD3ε(1-27)-MMP-2csV3-aCD3-aHER2-Xbody had higher Ec50s when not digested with MMP2 and Ec50s of less than 1 ng/mL when digested with MMP2. FIGS. 10A and 11A; Table 3. Consistent with data shown in FIGS. 7 and 8, these data suggest PABPs containing furin cleavage sites were cleaved by the HEK-293 cells, as expected, and that the PABPs containing the MMP2 cleavage sites were cleaved by MMP2 digestion. The non-cleavable CD3ε(1-27)-aCD3-aHER2-Xbody had an Ec50 comparable to that of undigested CD3ε(1-27)-MMP-2csV1-aCD3-aHER2-Xbody, regardless of whether it was digested with MMP2. Taken together, these data strongly suggest that presence of the CD3ε(1-27) fragment decreases the activity of PABPs in TDCC and T cell activation assays and that release of the CD3ε fragment by protease digestion increased the ability of PABPs to induce TDCC and T cell activation.

TABLE 3

Ec50’s

| Protein name | Ec50 Cytolysis (ng/mL) | |
|---|---|---|
| | -MMP2 | +MMP2 |
| aCD3-aHER2-Xbody | 0.1277 | ND |
| CD3ε(1-27)-aCD3-aHER2-Xbody | 6.115 | 6.731 |
| CD3ε(1-27)-FURINcsV1-aCD3-aHER2-Xbody | 0.2760 | 0.3316 |
| CD3ε(1-27)-FURINcsV2-aCD3-aHER2-Xbody | 0.1120 | 0.2563 |
| CD3ε(1-27)-MMP-2csV1-aCD3-aHER2-Xbody | 4.408 | 0.3469 |
| CD3ε(1-27)-MMP-2csV2-aCD3-aHER2-Xbody | 2.625 | 0.2200 |
| CD3ε(1-27)-MMP-2csV3-aCD3-aHER2-Xbody | 5.486 | 0.205 |
| aCD3-aHER2-mxb | 0.1845 | ND |

### Example 4: Cytolytic Activity of and T Cell Activation by scFv-Fc PABPs

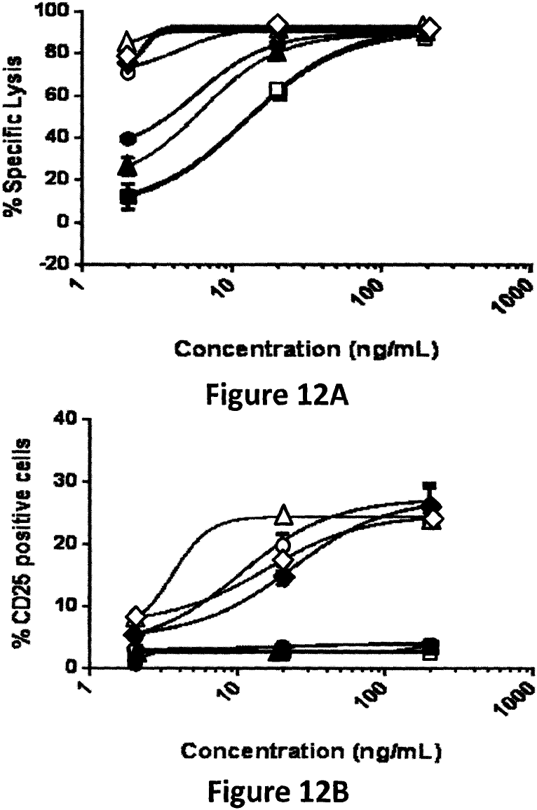

TDCC and T cell activation assays of anti-HER2/CD3 PABPs having the general structure shown in FIG. 4 were also performed. These PABPs have identical amino acid sequences except for the linker between the CD3ε fragment and the remainder of the molecule and included the following: CD3ε(1-27)-MMP-2csV1-aCD3-aHER2-mxb (comprising a linker with an MMP2 cleavage site), CD3ε(1-27)-MMP-2csV2-aCD3-aHER2-mxb (comprising a linker with a different MMP2 cleavage site), CD3ε(1-27)-FURINcsV2-aCD3-aHER2-mxb (comprising a linker with a furin cleavage site), and CD3ε(1-27)-aCD3-aHER2-mxb (comprising a non-cleavable linker). Results are shown in FIGS. 12A and 12B.

Samples of PABPs that would be expected to remain uncleaved, and thus retain the CD3ε fragment, showed low activity in the cytolysis assay and were not detectably active in the T cell activation assay. These samples included digested and undigested CD3ε(1-27)-aCD3-aHER2-mxb and undigested CD3ε(1-27)-MMP-2csV1-aCD3-aHER2-mxb and CD3ε(1-27)-MMP-2csV2-aCD3-aHER2-mxb. FIGS. 12A and 12B. In contrast, samples of PABPs that would be expected to be cleaved, and thus lack the CD3ε fragment, were much more active both assays. These samples included digested CD3ε(1-27)-MMP-2csV1-aCD3-aHER2-mxb and CD3ε(1-27)-MMP-2csV2-aCD3-aHER2-mxb and digested and undigested CD3ε(1-27)-FURINcsV2-aCD3-aHER2-mxb. FIGS. 12A and 12B. These data indicate that the presence of the CD3ε(1-27) fragment on these PABPs reduces their activity in TDCC and T cell activation assays and that these activities can be recovered upon proteolytic cleavage removing the CD3ε(1-27) fragment.

### Example 5: Binding of Bi-Fc PABPs to T Cells and Cytolytic Activity

PABPs having the format diagrammed in FIG. 2 were tested for binding to T cells and activity in a TDCC assay. Cytolytic activity was determined as described in Example 3 except that the target cells were JIMT-1 cells, which express about 181,000 molecules of HER2 protein per cell. Binding to T cells was assessed by fluorescence-activated cell sorting (FACS) analysis.

One PABP (CD3ε(1-27)-aCD3-aHER2-BiFc) contained a non-cleavable linker, one (CD3ε(1-27)-MMP-2cs-aCD3-aHER2-BiFc) contained an MMP2 cleavage site, and one (CD3ε(1-27)-FURINcs-aCD3-aHER2-BiFc) contained a furin cleavage site, which was expected to be cleaved intracellularly in the HEK-293 cells used to produce the proteins. A control protein (aCD3-aHER2-BiFc) had the format show in FIG. 2 except that did not contain the fragment of CD3ε. This molecule was expected to bind to T cells and to have cytolytic activity. An anti-CD3 IgG antibody was used as a positive control in the binding assay, and a sample containing no added protein was used as a negative control.

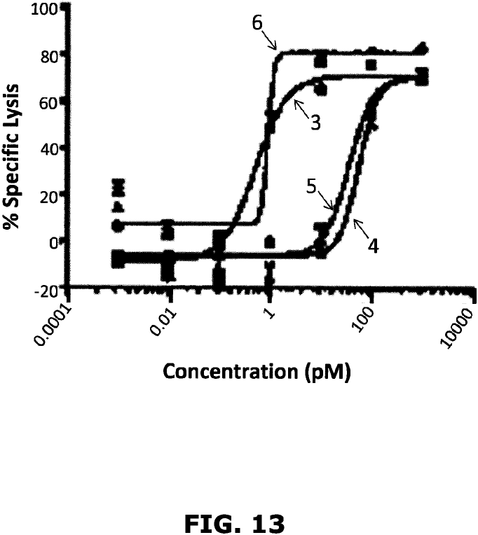

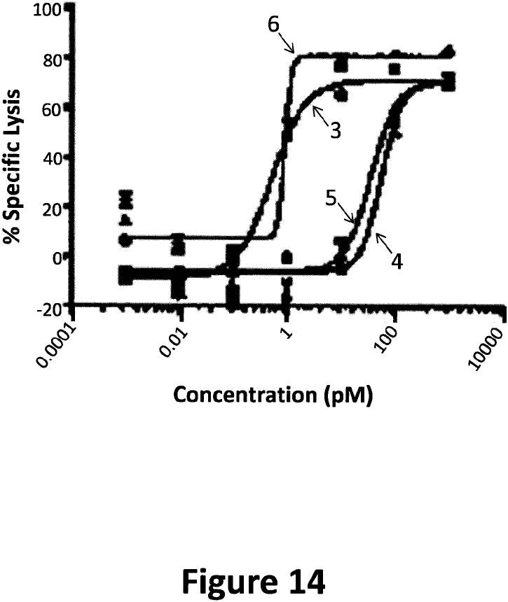

Consistent with these results, data in FIG. 13 indicate that CD3ε(1-27)-FURINcs-aCD3-aHER2-BiFc and aCD3-aHER2-BiFc (FIG. 13, lines labeled 6 and 3, respectively) have potent cytolytic activity, whereas CD3ε(1-27)-MMP-2cs-aCD3-aHER2-BiFc and CD3ε(1-27)-aCD3-aHER2-BiFc (FIG. 13, lines labeled 5 and 4, respectively) are considerably less active. These data suggest that presence of a fragment of CD3ε can prevent binding of these PABPs to T cells and substantially inhibit cytolytic activity.

### Sequence Listing

SEQ ID NO:1 Amino acid sequence of an MMP-2 cleavage site

GPLGIAGQ

SEQ ID NO:2 Amino acid sequence of an MMP-2 cleavage site

GGPLGMLSQS

SEQ ID NO:3 Amino acid sequence of an MMP-2 cleavage site

PLGLAG

SEQ ID NO:4 Amino acid sequence of a furin cleavage site

RRRRR

SEQ ID NO:5 Amino acid sequence of a furin cleavage site

GQSSRHRRAL

SEQ ID NO:6 Amino acid sequence of the first polypeptide chain of aCD3-aHER2-Xbody, CD3E(1-27)-aCD3-aHER2-Xbody, CD3ε(1-27)-MMP-2csV1-aCD3-aHER2-Xbody, CD3E(1-27)-MMP-2csV2-aCD3-aHER2-Xbody, CD3E(1-27)-MMP-2csV3-aCD3-aHER2-Xbody, CD3E(1-27)-FURINcsV1-aCD3-aHER2-Xbody, or CD3E(1-27)-FURINcsV2-aCD3-aHER2-Xbody (including signal sequence)

MGSTAILGLLLAVLQGGRAEVQLLEQSGAELVRPGALVKLSCKASGFKIK
DYFVNWVKQRPEQGLEWIGWIDPENDNSLYGPNFQDKASITADTSSNTGY
LQLSGLTSEDTAVYYCALYYGSRGDAMDYWGQGTTVTVSSGGGGSGGGGS
QTVVTQEPSLTVSPGGTVTLTCGSSTGAVTSGNYPNWVQQKPGQAPRGLI
GGTKFLAPGTPARFSGSLLGGKAALTLSGVQPEDEAEYYCVLWYSNRWVF
GGGTKLTVLGQPKAAPSVTLFPPSSEELQANKATLVCLISDFYPGAVTVA
WKADSSPVKAGVETTTPSKQSNNKYAASSYLSLTPEQWKSHRSYSCQVTH
EGSTVEKTVAPTECSAAAEPKSSDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRKEMTKNQVSLTCLVKGFYPSDIAVEWESNGQPENNYKTTPPV
LKSDGSFFLYSKLTVDKSRWQQGNVFSCSVMHEALHNHYTQKSLSLSPGK
AAAHHHHHH

SEQ ID NO:7 Nucleic acid sequence encoding SEQ ID NO:6

atggggtcaaccgccatccttggcctcctcctggctgtcctgcagggagg
gcgcgccgaggtgcagctgctcgagcagtctggagctgagcttgtgaggc
caggggccttagtcaagttgtcctgcaaagcttctggcttcaaaattaaa
gactactttgtgaactgggtgaagcagaggcctgaacagggcctggagtg
gattggatggattgatcctgagaatgataatagtttatatggcccgaact
tccaggacaaggccagtatcacagcagacacatcctccaacacaggctac
ctgcagctcagcggcctgacatctgaggacactgccgtctattactgtgc
tctttattacggaagtaggggggatgctatggactactggggccaaggga
ccacggtcaccgtctcctcaggaggcggcggttcaggcggaggtggctct
cagactgttgtgactcaggaaccttcactcaccgtatcacctggtggaac
agtcacactcacttgtggctcctcgactggggctgttacatctggcaact
acccaaactgggtccaacaaaaaccaggtcaggcaccccgtggtctaata
ggtgggactaagttcctcgcccccggtactcctgccagattctcaggctc
cctgcttggaggcaaggctgccctcaccctctcaggggtacagccagagg
atgaggcagaatattactgtgttctatggtacagcaaccgctgggtgttc
ggtggaggaaccaaactgactgtcctaggtcagcccaaggctgccccctc
ggtcactctgttcccgccctcctctgaggagcttcaagccaacaaggcca
cactggtgtgtctcataagtgacttctacccgggagccgtgacagtggcc
tggaaggcagatagcagccccgtcaaggcgggagtggagaccaccacacc
ctccaaacaaagcaacaacaagtacgcggccagcagctatctgagcctga
cgcctgagcagtggaagtcccacagaagctacagctgccaggtcacgcat
gaagggagcaccgtggagaagacagtggcccctacagaatgttcagcggc
cgcagagcccaaatcttctgacaaaactcacacatgccccccgtgcccag
cacctgaagcagctgggggaccgtcagtcttcctcttccccccaaaaccc
aaggacaccctcatgatctcccggacccctgaggtcacatgcgtggtggt
ggacgtgagccacgaagaccctgaggtcaagttcaactggtacgtggacg
gcgtggaggtgcataatgccaagacaaagccgcgagaggagcagtacaac
agcacgtaccgtgtggtcagcgtcctcaccgtcctgcaccaggactggct
gaatggcaaggagtacaagtgcaaggtctccaacaaagccctcccagccc
ccatcgagaaaaccatctccaaagccaaagggcagccccgagaaccacag
gtgtacaccctgcccccatcccggaaggagatgaccaagaaccaggtcag
cctgacctgcctggtcaaaggcttctatcccagcgacatcgccgtggagt
gggagagcaatgggcagccggagaacaactacaagaccacgcctcccgtg
ctgaagtccgacggctccttcttcctctatagcaagctcaccgtggacaa
gagcaggtggcagcaggggaacgtcttctcatgctccgtgatgcatgagg
ctctgcacaaccactacacgcagaagagcctctccctgtctccgggtaaa
gctgcagcgcatcaccaccaccatcac

SEQ ID NO:8 Amino acid sequence of the second polypeptide chain of CD3E(1-27)-aCD3-aHER2-Xbody (including signal sequence)

MGSTAILGLLLAVLQGGRAQDGNEEMGGITQTPYKVSISGTTVILTGGGG
SGGGGSGGGGSEVQLVESGGGLVQPGGSLKLSCAASGFTFNSYAMNWVRQ
APGKGLEWVARIRSKYNNYATYYADSVKGRFTISRDDSKNTAYLQMNNLK
TEDTAVYYCVRHGNFGNSYVSWWAYWGQGTLVTVSSGGGGSGGGGSELVM
TQTPSSLSASLGDRVTISCRASQDISNYLNWYQQKPDGTVKLLIYYTSRL
HSGVPSRFSGSGSGTDYSLTISNLEQEDIATYFCQQGNTLPLTFGAGTKL
EIKASTKGPSVFPLAPCSRSTSESTAALGCLVKDYFPEPVTVSWNSGALT
SGVHTFPAVLQSSGLYSLSSVVTVPSSNFGTQTYTCNVDHKPSNTKVDKT
VGGGGSAAAEPKSSDKTHTCPPCPAPEAAGGPSVFLFPPKPKDTLMISRT
PEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRE
EMTKNQVSLTCLVKGFYPSDIAVEWESNGQPENNYDTTPPVLDSDGSFFL
YSDLTVDKSRWQQGNVFSCSVMHEALHNHYTQKSLSLSPGK

SEQ ID NO:9 Nucleic acid sequence encoding SEQ ID NO:8

atggggtcaaccgccatccttggcctcctcctggctgtcctgcagggagg
gcgcgcccaggatggcaacgaagaaatgggcggcattacccagaccccgt
ataaagtgagcattagcggcaccaccgtgattctgaccggaggcggcggt
tcaggcggaggtggctctggcggtggcggaagtgaggtgcagctggtcga
gtctggaggcggattggtgcagcctggagggtcattgaaactctcatgtg
cagcctctggattcaccttcaatagctacgccatgaactgggtccgccag
gctccaggaaagggtttggaatgggttgctcgcataagaagtaaatataa
taattatgcaacatattatgccgattcagtgaaaggcaggttcaccatct
ccagagatgattcaaaaaacactgcctatctacaaatgaacaacttgaaa
actgaggacactgccgtgtactactgtgtgagacatgggaacttcggtaa
tagctacgtttcctggtgggcttactggggccaagggactctggtcaccg
tctcctcaggaggcggcggttcaggcggaggtggctctgagctcgtgatg
acccagactccatcctccctgtctgcctctctgggagacagagtcaccat
cagttgcagggcaagtcaggacattagcaattatttaaactggtatcagc
agaaaccagatggaactgttaaactcctgatctactacacatcaagatta
cactcaggagtcccatcaaggttcagtggcagtgggtctggaacagatta
ttctctcaccattagcaacctggagcaagaagatattgccacttactttt
gccaacagggtaatacgcttccgctcacgttcggtgctgggaccaagctt
gagatcaaagctagcaccaagggcccatcggtcttccccctggcgccctg
ctccaggagcacctccgagagcacagcggccctgggctgcctggtcaagg
actacttccccgaaccggtgacggtgtcgtggaactcaggcgctctgacc
agcggcgtgcacaccttcccagctgtcctacagtcctcaggactctactc
cctcagcagcgtggtgaccgtgccctccagcaacttcggcacccagacct
acacctgcaacgtagatcacaagcccagcaacaccaaggtggacaagaca
gttggcggaggtggctctgcggccgcagagcccaaatcttctgacaaaac
tcacacatgcccaccgtgcccagcacctgaagcagctgggggaccgtcag
tcttcctcttccccccaaaacccaaggacaccctcatgatctcccggacc
cctgaggtcacatgcgtggtggtggacgtgagccacgaagaccctgaggt
caagttcaactggtacgtggacggcgtggaggtgcataatgccaagacaa
agccgcgggaggagcagtacaacagcacgtaccgtgtggtcagcgtcctc
accgtcctgcaccaggactggctgaatggcaaggagtacaagtgcaaggt
ctccaacaaagccctcccagcccccatcgagaaaaccatctccaaagcca
aagggcagccccgagaaccacaggtgtacaccctgcccccatcccgggag
gagatgaccaagaaccaggtcagcctgacctgcctggtcaaaggcttcta
tcccagcgacatcgccgtggagtgggagagcaatgggcagccggagaaca
actacgacaccacgcctcccgtgctggactccgacggctccttcttcctc

SEQ ID NO:10 Amino acid sequence of the second polypeptide chain of CD3E(1-27)-MMP-2csV1-aCD3-aHER2-Xbody

MGSTAILGLLLAVLQGGRAQDGNEEMGGITQTPYKVSISGTTVILTGGGG
SGGGGSGGGGSGPLGIAGQEVQLVESGGGLVQPGGSLKLSCAASGFTFNS
YAMNWVRQAPGKGLEWVARIRSKYNNYATYYADSVKGRFTISRDDSKNTA
YLQMNNLKTEDTAVYYCVRHGNFGNSYVSWWAYWGQGTLVTVSSGGGGSG
GGGSELVMTQTPSSLSASLGDRVTISCRASQDISNYLNWYQQKPDGTVKL
LIYYTSRLHSGVPSRFSGSGSGTDYSLTISNLEQEDIATYFCQQGNTLPL
TFGAGTKLEIKASTKGPSVFPLAPCSRSTSESTAALGCLVKDYFPEPVTV
SWNSGALTSGVHTFPAVLQSSGLYSLSSVVTVPSSNFGTQTYTCNVDHKP
SNTKVDKTVGGGGSAAAEPKSSDKTHTCPPCPAPEAAGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYDTTPPVL
DSDGSFFLYSDLTVDKSRWQQGNVFSCSVMHEALHNHYTQKSLSLSPGK

SEQ ID NO:11 Nucleic acid sequence encoding SEQ ID NO:10

atggggtcaaccgccatccttggcctcctcctggctgtcctgcagggagg
gcgcgcccaggatggcaacgaagaaatgggcggcattacccagaccccgt
ataaagtgagcattagcggcaccaccgtgattctgaccggaggcggcggt
tcaggcggaggtggctctggcggtggcggaagtggaccgttgggtatcgc
tggccaggaggtgcagctggtcgagtctggaggaggattggtgcagcctg
gagggtcattgaaactctcatgtgcagcctctggattcaccttcaatagc
tacgccatgaactgggtccgccaggctccaggaaagggtttggaatgggt
tgctcgcataagaagtaaatataataattatgcaacatattatgccgatt
cagtgaaaggcaggttcaccatctccagagatgattcaaaaaacactgcc
tatctacaaatgaacaacttgaaaactgaggacactgccgtgtactactg
tgtgagacatgggaacttcggtaatagctacgtttcctggtgggcttact
ggggccaagggactctggtcaccgtctcctcaggaggcggcggttcaggc
ggaggtggctctgagctcgtgatgacccagactccatcctccctgtctgc
ctctctgggagacagagtcaccatcagttgcagggcaagtcaggacatta
gcaattatttaaactggtatcagcagaaaccagatggaactgttaaactc
ctgatctactacacatcaagattacactcaggagtcccatcaaggttcag
tggcagtgggtctggaacagattattctctcaccattagcaacctggagc
aagaagatattgccacttacttttgccaacagggtaatacgcttccgctc
acgttcggtgctgggaccaagcttgagatcaaagctagcaccaagggccc
atcggtcttccccctggcgccctgctccaggagcacctccgagagcacag
cggccctgggctgcctggtcaaggactacttccccgaaccggtgacggtg
tcgtggaactcaggcgctctgaccagcggcgtgcacaccttcccagctgt
cctacagtcctcaggactctactccctcagcagcgtggtgaccgtgccct
ccagcaacttcggcacccagacctacacctgcaacgtagatcacaagccc
agcaacaccaaggtggacaagacagttggcggaggtggctctgcggccgc
agagcccaaatcttctgacaaaactcacacatgcccaccgtgcccagcac
ctgaagcagctgggggaccgtcagtcttcctcttccccccaaaacccaag
gacaccctcatgatctcccggacccctgaggtcacatgcgtggtggtgga
cgtgagccacgaagaccctgaggtcaagttcaactggtacgtggacggcg
tggaggtgcataatgccaagacaaagccgcgggaggagcagtacaacagc
acgtaccgtgtggtcagcgtcctcaccgtcctgcaccaggactggctgaa
tggcaaggagtacaagtgcaaggtctccaacaaagccctcccagccccca
tcgagaaaaccatctccaaagccaaagggcagccccgagaaccacaggtg
tacaccctgcccccatcccgggaggagatgaccaagaaccaggtcagcct
gacctgcctggtcaaaggcttctatcccagcgacatcgccgtggagtggg
agagcaatgggcagccggagaacaactacgacaccacgcctcccgtgctg
gactccgacggctccttcttcctctatagcgacctcaccgtggacaagag
caggtggcagcaggggaacgtcttctcatgctccgtgatgcatgaggctc
tgcacaaccactacacgcagaagagcctctccctgtctccgggtaaa

SEQ ID NO:12 Amino acid sequence of the second polypeptide chain of CD3E(1-27)-MMP-2csV2-aCD3-aHER2-Xbody

MGSTAILGLLLAVLQGGRAQDGNEEMGGITQTPYKVSISGTTVILTGGGG
SGGGGSGGGGSGGPLGMLSQSEVQLVESGGGLVQPGGSLKLSCAASGFTF
NSYAMNWVRQAPGKGLEWVARIRSKYNNYATYYADSVKGRFTISRDDSKN
TAYLQMNNLKTEDTAVYYCVRHGNFGNSYVSWWAYWGQGTLVTVSSGGGG
SGGGGSELVMTQTPSSLSASLGDRVTISCRASQDISNYLNWYQQKPDGTV
KLLIYYTSRLHSGVPSRFSGSGSGTDYSLTISNLEQEDIATYFCQQGNTL
PLTFGAGTKLEIKASTKGPSVFPLAPCSRSTSESTAALGCLVKDYFPEPV
TVSWNSGALTSGVHTFPAVLQSSGLYSLSSWTVPSSNFGTQTYTCNVDHK
PSNTKVDKTVGGGGSAAAEPKSSDKTHTCPPCPAPEAAGGPSVFLFPPKP
KDTLMISRTPEVTCVWDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYDTTPPVL
DSDGSFFLYSDLTVDKSRWQQGNVFSCSVMHEALHNHYTQKSLSLSPGK

SEQ ID NO:13 Nucleic acid sequence encoding SEQ ID NO:12

atggggtcaaccgccatccttggcctcctcctggctgtcctgcagggagg
gcgcgcccaggatggcaacgaagaaatgggcggcattacccagaccccgt
ataaagtgagcattagcggcaccaccgtgattctgaccggaggcggcggt
tcaggcggaggtggctctggcggtggcggaagtggtggacctttgggtat
gcttagtcagagcgaggtgcagctggtcgagtctggaggaggattggtgc
agcctggagggtcattgaaactctcatgtgcagcctctggattcaccttc
aatagctacgccatgaactgggtccgccaggctccaggaaagggtttgga
atgggttgctcgcataagaagtaaatataataattatgcaacatattatg
ccgattcagtgaaaggcaggttcaccatctccagagatgattcaaaaaac
actgcctatctacaaatgaacaacttgaaaactgaggacactgccgtgta
ctactgtgtgagacatgggaacttcggtaatagctacgtttcctggtggg
cttactggggccaagggactctggtcaccgtctcctcaggaggcggcggt
tcaggcggaggtggctctgagctcgtgatgacccagactccatcctccct
gtctgcctctctgggagacagagtcaccatcagttgcagggcaagtcagg
acattagcaattatttaaactggtatcagcagaaaccagatggaactgtt
aaactcctgatctactacacatcaagattacactcaggagtcccatcaag
gttcagtggcagtgggtctggaacagattattctctcaccattagcaacc
tggagcaagaagatattgccacttacttttgccaacagggtaatacgctt
ccgctcacgttcggtgctgggaccaagcttgagatcaaagctagcaccaa
gggcccatcggtcttccccctggcgccctgctccaggagcacctccgaga
gcacagcggccctgggctgcctggtcaaggactacttccccgaaccggtg
acggtgtcgtggaactcaggcgctctgaccagcggcgtgcacaccttccc
agctgtcctacagtcctcaggactctactccctcagcagcgtggtgaccg
tgccctccagcaacttcggcacccagacctacacctgcaacgtagatcac
aagcccagcaacaccaaggtggacaagacagttggcggaggtggctctgc
ggccgcagagcccaaatcttctgacaaaactcacacatgcccaccgtgcc
cagcacctgaagcagctgggggaccgtcagtcttcctcttccccccaaaa
cccaaggacaccctcatgatctcccggacccctgaggtcacatgcgtggt
ggtggacgtgagccacgaagaccctgaggtcaagttcaactggtacgtgg
acggcgtggaggtgcataatgccaagacaaagccgcgggaggagcagtac
aacagcacgtaccgtgtggtcagcgtcctcaccgtcctgcaccaggactg
gctgaatggcaaggagtacaagtgcaaggtctccaacaaagccctcccag
cccccatcgagaaaaccatctccaaagccaaagggcagccccgagaacca
caggtgtacaccctgcccccatcccgggaggagatgaccaagaaccaggt
cagcctgacctgcctggtcaaaggcttctatcccagcgacatcgccgtgg
agtgggagagcaatgggcagccggagaacaactacgacaccacgcctccc
gtgctggactccgacggctccttcttcctctatagcgacctcaccgtgga
caagagcaggtggcagcaggggaacgtcttctcatgctccgtgatgcatg
aggctctgcacaaccactacacgcagaagagcctctccctgtctccgggt
aaa

SEQ ID NO:14 Amino acid sequence of the second polypeptide chain of CD3E(1-27)-MMP-2csV3-aCD3-aHER2-Xbody

MGSTAILGLLLAVLQGGRAQDGNEEMGGITQTPYKVSISGTTVILTGGGG
SGGGGSGGGGSPLGLAGEVQLVESGGGLVQPGGSLKLSCAASGFTFNSYA
MNWVRQAPGKGLEWVARIRSKYNNYATYYADSVKGRFTISRDDSKNTAYL
QMNNLKTEDTAVYYCVRHGNFGNSYVSWWAYWGQGTLVTVSSGGGGSGGG
GSELVMTQTPSSLSASLGDRVTISCRASQDISNYLNWYQQKPDGTVKLLI
YYTSRLHSGVPSRFSGSGSGTDYSLTISNLEQEDIATYFCQQGNTLPLTF
GAGTKLEIKASTKGPSVFPLAPCSRSTSESTAALGCLVKDYFPEPVTVSW
NSGALTSGVHTFPAVLQSSGLYSLSSVVTVPSSNFGTQTYTCNVDHKPSN
TKVDKTVGGGGSAAAEPKSSDKTHTCPPCPAPEAAGGPSVFLFPPKPKDT
LMISRTPEVTCVWDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYDTTPPVLDSD
GSFFLYSDLTVDKSRWQQGNVFSCSVMHEALHNHYTQKSLSLSPGK

SEQ ID NO:15 Nucleic acid sequence encoding SEQ ID NO:14

atggggtcaaccgccatccttggcctcctcctggctgtcctgcagggagg
gcgcgcccaggatggcaacgaagaaatgggcggcattacccagaccccgt
ataaagtgagcattagcggcaccaccgtgattctgaccggaggcggcggt
tcaggcggaggtggctctggcggtggcggaagtcctttgggtcttgccgg
agaggtgcagctggtcgagtctggaggcggattggtgcagcctggagggt
cattgaaactctcatgtgcagcctctggattcaccttcaatagctacgcc
atgaactgggtccgccaggctccaggaaagggtttggaatgggttgctcg
cataagaagtaaatataataattatgcaacatattatgccgattcagtga
aaggcaggttcaccatctccagagatgattcaaaaaacactgcctatcta
caaatgaacaacttgaaaactgaggacactgccgtgtactactgtgtgag
acatgggaacttcggtaatagctacgtttcctggtgggcttactggggcc
aagggactctggtcaccgtctcctcaggaggcggcggttcaggcggaggt
ggctctgagctcgtgatgacccagactccatcctccctgtctgcctctct
gggagacagagtcaccatcagttgcagggcaagtcaggacattagcaatt
atttaaactggtatcagcagaaaccagatggaactgttaaactcctgatc
tactacacatcaagattacactcaggagtcccatcaaggttcagtggcag
tgggtctggaacagattattctctcaccattagcaacctggagcaagaag
atattgccacttacttttgccaacagggtaatacgcttccgctcacgttc
ggtgctgggaccaagcttgagatcaaagctagcaccaagggcccatcggt
cttccccctggcgccctgctccaggagcacctccgagagcacagcggccc
tgggctgcctggtcaaggactacttccccgaaccggtgacggtgtcgtgg
aactcaggcgctctgaccagcggcgtgcacaccttcccagctgtcctaca
gtcctcaggactctactccctcagcagcgtggtgaccgtgccctccagca
acttcggcacccagacctacacctgcaacgtagatcacaagcccagcaac
accaaggtggacaagacagttggcggaggtggctctgcggccgcagagcc
caaatcttctgacaaaactcacacatgcccaccgtgcccagcacctgaag
cagctgggggaccgtcagtcttcctcttccccccaaaacccaaggacacc
ctcatgatctcccggacccctgaggtcacatgcgtggtggtggacgtgag
ccacgaagaccctgaggtcaagttcaactggtacgtggacggcgtggagg
tgcataatgccaagacaaagccgcgggaggagcagtacaacagcacgtac
cgtgtggtcagcgtcctcaccgtcctgcaccaggactggctgaatggcaa
ggagtacaagtgcaaggtctccaacaaagccctcccagcccccatcgaga
aaaccatctccaaagccaaagggcagccccgagaaccacaggtgtacacc
ctgcccccatcccgggaggagatgaccaagaaccaggtcagcctgacctg
cctggtcaaaggcttctatcccagcgacatcgccgtggagtgggagagca
atgggcagccggagaacaactacgacaccacgcctcccgtgctggactcc
gacggctccttcttcctctatagcgacctcaccgtggacaagagcaggtg
gcagcaggggaacgtcttctcatgctccgtgatgcatgaggctctgcaca
accactacacgcagaagagcctctccctgtctccgggtaaa

SEQ ID NO:16 Amino acid sequence of the second polypeptide chain of CD3E(1-27)-FURINcsV1-aCD3-aHER2-Xbody

MGSTAILGLLLAVLQGGRAQDGNEEMGGITQTPYKVSISGTTVILTGGGG
SGGGGSGGGGSRRRRREVQLVESGGGLVQPGGSLKLSCAASGFTFNSYAM
NWVRQAPGKGLEWVARIRSKYNNYATYYADSVKGRFTISRDDSKNTAYLQ
MNNLKTEDTAVYYCVRHGNFGNSYVSWWAYWGQGTLVTVSSGGGGSGGGG
SELVMTQTPSSLSASLGDRVTISCRASQDISNYLNWYQQKPDGTVKLLIY
YTSRLHSGVPSRFSGSGSGTDYSLTISNLEQEDIATYFCQQGNTLPLTFG
AGTKLEIKASTKGPSVFPLAPCSRSTSESTAALGCLVKDYFPEPVTVSWN
SGALTSGVHTFPAVLQSSGLYSLSSVVTVPSSNFGTQTYTCNVDHKPSNT
KVDKTVGGGGSAAAEPKSSDKTHTCPPCPAPEAAGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYDTTPPVLDSD
GSFFLYSDLTVDKSRWQQGNVFSCSVMHEALHNHYTQKSLSLSPGK

SEQ ID NO:17 Nucleic acid sequence encoding SEQ ID NO:16

atggggtcaaccgccatccttggcctcctcctggctgtcctgcagggagg
gcgcgcccaggatggcaacgaagaaatgggcggcattacccagaccccgt
ataaagtgagcattagcggcaccaccgtgattctgaccggaggcggcggt
tcaggcggaggtggctctggcggtggcggaagtcggcgaagacgtcgcga
ggtgcagctggtcgagtctggaggaggattggtgcagcctggagggtcat
tgaaactctcatgtgcagcctctggattcaccttcaatagctacgccatg
aactgggtccgccaggctccaggaaagggtttggaatgggttgctcgcat
aagaagtaaatataataattatgcaacatattatgccgattcagtgaaag
gcaggttcaccatctccagagatgattcaaaaaacactgcctatctacaa
atgaacaacttgaaaactgaggacactgccgtgtactactgtgtgagaca
tgggaacttcggtaatagctacgtttcctggtgggcttactggggccaag
ggactctggtcaccgtctcctcaggaggcggcggttcaggcggaggtggc
tctgagctcgtgatgacccagactccatcctccctgtctgcctctctggg
agacagagtcaccatcagttgcagggcaagtcaggacattagcaattatt
taaactggtatcagcagaaaccagatggaactgttaaactcctgatctac
tacacatcaagattacactcaggagtcccatcaaggttcagtggcagtgg
gtctggaacagattattctctcaccattagcaacctggagcaagaagata
ttgccacttacttttgccaacagggtaatacgcttccgctcacgttcggt
gctgggaccaagcttgagatcaaagctagcaccaagggcccatcggtctt
ccccctggcgccctgctccaggagcacctccgagagcacagcggccctgg
gctgcctggtcaaggactacttccccgaaccggtgacggtgtcgtggaac
tcaggcgctctgaccagcggcgtgcacaccttcccagctgtcctacagtc
ctcaggactctactccctcagcagcgtggtgaccgtgccctccagcaact
tcggcacccagacctacacctgcaacgtagatcacaagcccagcaacacc
aaggtggacaagacagttggcggaggtggctctgcggccgcagagcccaa
atcttctgacaaaactcacacatgcccaccgtgcccagcacctgaagcag
ctgggggaccgtcagtcttcctcttccccccaaaacccaaggacaccctc
atgatctcccggacccctgaggtcacatgcgtggtggtggacgtgagcca
cgaagaccctgaggtcaagttcaactggtacgtggacggcgtggaggtgc
ataatgccaagacaaagccgcgggaggagcagtacaacagcacgtaccgt
gtggtcagcgtcctcaccgtcctgcaccaggactggctgaatggcaagga
gtacaagtgcaaggtctccaacaaagccctcccagcccccatcgagaaaa
ccatctccaaagccaaagggcagccccgagaaccacaggtgtacaccctg
cccccatcccgggaggagatgaccaagaaccaggtcagcctgacctgcct
ggtcaaaggcttctatcccagcgacatcgccgtggagtgggagagcaatg
ggcagccggagaacaactacgacaccacgcctcccgtgctggactccgac
ggctccttcttcctctatagcgacctcaccgtggacaagagcaggtggca
gcaggggaacgtcttctcatgctccgtgatgcatgaggctctgcacaacc
actacacgcagaagagcctctccctgtctccgggtaaa

SEQ ID NO:18 Amino acid sequence of the second polypeptide chain of CD3E(1-27)-FURINcsV2-aCD3-aHER2-Xbody

MGSTAIFGLLLAVLQGGRAQDGNEEMGGITQTPYKVSISGTTVILTGGGG
SGGGGSGGGGSGQSSRHRRALEVQLVESGGGLVQPGGSLKLSCAASGFTF
NSYAMNWVRQAPGKGLEWVARIRSKYNNYATYYADSVKGRFTISRDDSKN
TAYLQMNNLKTEDTAVYYCVRHGNFGNSYVSWWAYWGQGTLVTVSSGGGG
SGGGGSELVMTQTPSSLSASLGDRVTISCRASQDISNYLNWYQQKPDGTV
KLLIYYTSRLHSGVPSRFSGSGSGTDYSLTISNLEQEDIATYFCQQGNTL
PLTFGAGTKLEIKASTKGPSVFPLAPCSRSTSESTAALGCLVKDYFPEPV
TVSWNSGALTSGVHTFPAVLQSSGLYSLSSWTVPSSNFGTQTYTCNVDHK
PSNTKVDKTVGGGGSAAAEPKSSDKTHTCPPCPAPEAAGGPSVFLFPPKP
KDTLMISRTPEVTCVWDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYDTTPPVL
DSDGSFFLYSDLTVDKSRWQQGNVFSCSVMHEALHNHYTQKSLSLSPGK

SEQ ID NO:19 Nucleic acid sequence encoding SEQ ID NO:18

atggggtcaaccgccatctttggcctcctcctggctgtcctgcagggagg
gcgcgcccaggatggcaacgaagaaatgggcggcattacccagaccccgt
ataaagtgagcattagcggcaccaccgtgattctgaccggaggcggcggt
tcaggcggaggtggctctggcggtggcggaagtggtcagagtagccgaca
cagacgtgcactagaggtgcagctggtcgagtctggaggaggattggtgc
agcctggagggtcattgaaactctcatgtgcagcctctggattcaccttc
aatagctacgccatgaactgggtccgccaggctccaggaaagggtttgga
atgggttgctcgcataagaagtaaatataataattatgcaacatattatg
ccgattcagtgaaaggcaggttcaccatctccagagatgattcaaaaaac
actgcctatctacaaatgaacaacttgaaaactgaggacactgccgtgta
ctactgtgtgagacatgggaacttcggtaatagctacgtttcctggtggg
cttactggggccaagggactctggtcaccgtctcctcaggaggcggcggt
tcaggcggaggtggctctgagctcgtgatgacccagactccatcctccct
gtctgcctctctgggagacagagtcaccatcagttgcagggcaagtcagg
acattagcaattatttaaactggtatcagcagaaaccagatggaactgtt
aaactcctgatctactacacatcaagattacactcaggagtcccatcaag
gttcagtggcagtgggtctggaacagattattctctcaccattagcaacc
tggagcaagaagatattgccacttacttttgccaacagggtaatacgctt
ccgctcacgttcggtgctgggaccaagcttgagatcaaagctagcaccaa
gggcccatcggtcttccccctggcgccctgctccaggagcacctccgaga
gcacagcggccctgggctgcctggtcaaggactacttccccgaaccggtg
acggtgtcgtggaactcaggcgctctgaccagcggcgtgcacaccttccc
agctgtcctacagtcctcaggactctactccctcagcagcgtggtgaccg
tgccctccagcaacttcggcacccagacctacacctgcaacgtagatcac
aagcccagcaacaccaaggtggacaagacagttggcggaggtggctctgc
ggccgcagagcccaaatcttctgacaaaactcacacatgcccaccgtgcc
cagcacctgaagcagctgggggaccgtcagtcttcctcttccccccaaaa
cccaaggacaccctcatgatctcccggacccctgaggtcacatgcgtggt
ggtggacgtgagccacgaagaccctgaggtcaagttcaactggtacgtgg
acggcgtggaggtgcataatgccaagacaaagccgcgggaggagcagtac
aacagcacgtaccgtgtggtcagcgtcctcaccgtcctgcaccaggactg
gctgaatggcaaggagtacaagtgcaaggtctccaacaaagccctcccag
cccccatcgagaaaaccatctccaaagccaaagggcagccccgagaacca
caggtgtacaccctgcccccatcccgggaggagatgaccaagaaccaggt
cagcctgacctgcctggtcaaaggcttctatcccagcgacatcgccgtgg
agtgggagagcaatgggcagccggagaacaactacgacaccacgcctccc
gtgctggactccgacggctccttcttcctctatagcgacctcaccgtgga
caagagcaggtggcagcaggggaacgtcttctcatgctccgtgatgcatg
aggctctgcacaaccactacacgcagaagagcctctccctgtctccgggt
aaa

SEQ ID NO:20 Amino acid sequence of the first polypeptide chain of aCD3-aHER2-mxb, CD3E(1-27)-aCD3-aHER2-mxb, CD3E(1-27)-MMP-2csV1-aCD3-aHER2-mxb, CD3E(1-27)-MMP-2csV2-aCD3-aHER2-mxb

MGSTAILGLLLAVLQGGRAEVQLLEQSGAELVRPGALVKLSCKASGFKIK
DYFVNWVKQRPEQGLEWIGWIDPENDNSLYGPNFQDKASITADTSSNTGY
LQLSGLTSEDTAVYYCALYYGSRGDAMDYWGQGTTVTVSSGGGGSGGGGS
GGGGSELVMTQTPSSLSASLGDRVTISCRASQDISNYLNWYQQKPDGTVK
LLIYYTSRLHSGVPSRFSGSGSGTDYSLTISNLEQEDIATYFCQQGNTLP
LTFGAGTKLEIKAAAEPKSSDKTHTCPPCPAPEAAGGPSVFLFPPKPKDT
LMISRTPEVTCVWDVSHEDPEVKFNWYVDGVEVHNAKTKPREEQYNSTYR
VVSVLTVLHQDWLNGKEYKCKVSNKALPAPIEKTISKAKGQPREPQVYTL
PPSRKEMTKNQVSLTCLVKGFYPSDIAVEWESNGQPENNYKTTPPVLKSD
GSFFLYSKLTVDKSRWQQGNVFSCSVMHEALHNHYTQKSLSLSPGKAAAH
HHHHH

SEQ ID NO:21 Nucleic acid sequence encoding SEQ ID NO:20

atggggtcaaccgccatccttggcctcctcctggctgtcctgcagggagg
gcgcgccgaggtgcagctgctcgagcagtctggagctgagcttgtgaggc
caggggccttagtcaagttgtcctgcaaagcttctggcttcaaaattaaa
gactactttgtgaactgggtgaagcagaggcctgaacagggcctggagtg
gattggatggattgatcctgagaatgataatagtttatatggcccgaact
tccaggacaaggccagtatcacagcagacacatcctccaacacaggctac
ctgcagctcagcggcctgacatctgaggacactgccgtctattactgtgc
tctttattacggaagtaggggggatgctatggactactggggccaaggga
ccacggtcaccgtctcctcaggtggtggtggttctggcggcggcggctcc
ggtggtggtggttctgagctcgtgatgacccagactccatcctccctgtc
tgcctctctgggagacagagtcaccatcagttgcagggcaagtcaggaca
ttagcaattatttaaactggtatcagcagaaaccagatggaactgttaaa
ctcctgatctactacacatcaagattacactcaggagtcccatcaaggtt
cagtggcagtgggtctggaacagattattctctcaccattagcaacctgg
agcaagaagatattgccacttacttttgccaacagggtaatacgcttccg
ctcacgttcggtgctgggaccaagcttgagatcaaagcggccgcagagcc
caaatcttctgacaaaactcacacatgccccccgtgcccagcacctgaag
cagctgggggaccgtcagtcttcctcttccccccaaaacccaaggacacc
ctcatgatctcccggacccctgaggtcacatgcgtggtggtggacgtgag
ccacgaagaccctgaggtcaagttcaactggtacgtggacggcgtggagg
tgcataatgccaagacaaagccgcgagaggagcagtacaacagcacgtac
cgtgtggtcagcgtcctcaccgtcctgcaccaggactggctgaatggcaa
ggagtacaagtgcaaggtctccaacaaagccctcccagcccccatcgaga
aaaccatctccaaagccaaagggcagccccgagaaccacaggtgtacacc
ctgcccccatcccggaaggagatgaccaagaaccaggtcagcctgacctg
cctggtcaaaggcttctatcccagcgacatcgccgtggagtgggagagca
atgggcagccggagaacaactacaagaccacgcctcccgtgctgaagtcc
gacggctccttcttcctctatagcaagctcaccgtggacaagagcaggtg
gcagcaggggaacgtcttctcatgctccgtgatgcatgaggctctgcaca
accactacacgcagaagagcctctccctgtctccgggtaaagctgcagcg
catcaccaccaccatcac

SEQ ID NO:22 Amino acid sequence of the second polypeptide chain of CD3E(1-27)-aCD3-aHER2-mxb

MGSTAILGLLLAVLQGGRAQDGNEEMGGITQTPYKVSISGTTVILTGGGG
SGGGGSGGGGSEVQLVESGGGLVQPGGSLKLSCAASGFTFNSYAMNWVRQ
APGKGLEWVARIRSKYNNYATYYADSVKGRFTISRDDSKNTAYLQMNNLK
TEDTAVYYCVRHGNFGNSYVSWWAYWGQGTLVTVSSGGGGSGGGGSGGGG
SQTVVTQEPSLTVSPGGTVTLTCGSSTGAVTSGNYPNWVQQKPGQAPRGL
IGGTKFLAPGTPARFSGSLLGGKAALTLSGVQPEDEAEYYCVLWYSNRWV
FGGGTKLTVLAAAEPKSSDKTHTCPPCPAPEAAGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSREEMTKNQVSLTCLVKGFYPSDIAVEWESNGQPENNYDTTPPVLDSDG
SFFLYSDLTVDKSRWQQGNVFSCSVMHEALHNHYTQKSLSLSPGK

SEQE ID NO:23 Nucleic acid sequence encoding SEQ ID NO:22

atggggtcaaccgccatccttggcctcctcctggctgtcctgcagggagg
gcgcgcccaggatggcaacgaagaaatgggcggcattacccagaccccgt
ataaagtgagcattagcggcaccaccgtgattctgaccggaggcggcggt
tcaggcggaggtggctctggcggtggcggaagtgaggtgcagctggtcga
gtctggaggaggattggtgcagcctggagggtcattgaaactctcatgtg
cagcctctggattcaccttcaatagctacgccatgaactgggtccgccag
gctccaggaaagggtttggaatgggttgctcgcataagaagtaaatataa
taattatgcaacatattatgccgattcagtgaaaggcaggttcaccatct
ccagagatgattcaaaaaacactgcctatctacaaatgaacaacttgaaa
actgaggacactgccgtgtactactgtgtgagacatgggaacttcggtaa
tagctacgtttcctggtgggcttactggggccaagggactctggtcaccg
tctcctcaggtggtggtggttctggcggcggcggctccggtggtggtggt
tctcagactgttgtgactcaggaaccttcactcaccgtatcacctggtgg
aacagtcacactcacttgtggctcctcgactggggctgttacatctggca
actacccaaactgggtccaacaaaaaccaggtcaggcaccccgtggtcta
ataggtgggactaagttcctcgcccccggtactcctgccagattctcagg
ctccctgcttggaggcaaggctgccctcaccctctcaggggtacagccag
aggatgaggcagaatattactgtgttctatggtacagcaaccgctgggtg
ttcggtggaggaaccaaactgactgtcctagcggccgcagagcccaaatc
ttctgacaaaactcacacatgcccaccgtgcccagcacctgaagcagctg
ggggaccgtcagtcttcctcttccccccaaaacccaaggacaccctcatg
atctcccggacccctgaggtcacatgcgtggtggtggacgtgagccacga
agaccctgaggtcaagttcaactggtacgtggacggcgtggaggtgcata
atgccaagacaaagccgcgggaggagcagtacaacagcacgtaccgtgtg
gtcagcgtcctcaccgtcctgcaccaggactggctgaatggcaaggagta
caagtgcaaggtctccaacaaagccctcccagcccccatcgagaaaacca
tctccaaagccaaagggcagccccgagaaccacaggtgtacaccctgccc
ccatcccgggaggagatgaccaagaaccaggtcagcctgacctgcctggt
caaaggcttctatcccagcgacatcgccgtggagtgggagagcaatgggc
agccggagaacaactacgacaccacgcctcccgtgctggactccgacggc
tccttcttcctctatagcgacctcaccgtggacaagagcaggtggcagca
ggggaacgtcttctcatgctccgtgatgcatgaggctctgcacaaccact
acacgcagaagagcctctccctgtctccgggtaaa

SEQ ID NO:24 Amino acid sequence of the second polypeptide chain of CD3E(1-27)-MMP-2csV1-aCD3-aHER2-mxb

MGSTAILGLLLAVLQGGRAQDGNEEMGGITQTPYKVSISGTTVILTGGGG
SGGGGSGGGGSGPLGIAGQEVQLVESGGGLVQPGGSLKLSCAASGFTFNS
YAMNWVRQAPGKGLEWVARIRSKYNNYATYYADSVKGRFTISRDDSKNTA
YLQMNNLKTEDTAVYYCVRHGNFGNSYVSWWAYWGQGTLVTVSSGGGGSG
GGGSGGGGSQTVVTQEPSLTVSPGGTVTLTCGSSTGAVTSGNYPNWVQQK
PGQAPRGLIGGTKFLAPGTPARFSGSLLGGKAALTLSGVQPEDEAEYYCV
LWYSNRWVFGGGTKLTVLAAAEPKSSDKTHTCPPCPAPEAAGGPSVFLFP
PKPKDTLMISRTPEVTCVWDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYDTTP
PVLDSDGSFFLYSDLTVDKSRWQQGNVFSCSVMHEALHNHYTQKSLSLSP
GK

SEQ ID NO:25 Nucleic acid encoding SEQ ID NO:24

atggggtcaaccgccatccttggcctcctcctggctgtcctgcagggagg
gcgcgcccaggatggcaacgaagaaatgggcggcattacccagaccccgt
ataaagtgagcattagcggcaccaccgtgattctgaccggaggcggcggt
tcaggcggaggtggctctggcggtggcggaagtggaccgttgggtatcgc
tggccaggaggtgcagctggtcgagtctggaggaggattggtgcagcctg
gagggtcattgaaactctcatgtgcagcctctggattcaccttcaatagc
tacgccatgaactgggtccgccaggctccaggaaagggtttggaatgggt
tgctcgcataagaagtaaatataataattatgcaacatattatgccgatt
cagtgaaaggcaggttcaccatctccagagatgattcaaaaaacactgcc
tatctacaaatgaacaacttgaaaactgaggacactgccgtgtactactg
tgtgagacatgggaacttcggtaatagctacgtttcctggtgggcttact
ggggccaagggactctggtcaccgtctcctcaggtggtggtggttctggc
ggcggcggctccggtggtggtggttctcagactgttgtgactcaggaacc
ttcactcaccgtatcacctggtggaacagtcacactcacttgtggctcct
cgactggggctgttacatctggcaactacccaaactgggtccaacaaaaa
ccaggtcaggcaccccgtggtctaataggtgggactaagttcctcgcccc
cggtactcctgccagattctcaggctccctgcttggaggcaaggctgccc
tcaccctctcaggggtacagccagaggatgaggcagaatattactgtgtt
ctatggtacagcaaccgctgggtgttcggtggaggaaccaaactgactgt
cctagcggccgcagagcccaaatcttctgacaaaactcacacatgcccac
cgtgcccagcacctgaagcagctgggggaccgtcagtcttcctcttcccc
ccaaaacccaaggacaccctcatgatctcccggacccctgaggtcacatg
cgtggtggtggacgtgagccacgaagaccctgaggtcaagttcaactggt
acgtggacggcgtggaggtgcataatgccaagacaaagccgcgggaggag
cagtacaacagcacgtaccgtgtggtcagcgtcctcaccgtcctgcacca
ggactggctgaatggcaaggagtacaagtgcaaggtctccaacaaagccc
tcccagcccccatcgagaaaaccatctccaaagccaaagggcagccccga
gaaccacaggtgtacaccctgcccccatcccgggaggagatgaccaagaa
ccaggtcagcctgacctgcctggtcaaaggcttctatcccagcgacatcg
ccgtggagtgggagagcaatgggcagccggagaacaactacgacaccacg
cctcccgtgctggactccgacggctccttcttcctctatagcgacctcac
cgtggacaagagcaggtggcagcaggggaacgtcttctcatgctccgtga
tgcatgaggctctgcacaaccactacacgcagaagagcctctccctgtct
ccgggtaaa

SEQ ID NO:26 Amino acid sequence of the second polypeptide chain of CD3E(1-27)-MMP-2csV2-aCD3-aHER2-mxb

MGSTAILGLLLAVLQGGRAQDGNEEMGGITQTPYKVSISGTTVILTGGGG
SGGGGSGGGGSGGPLGMLSQSEVQLVESGGGLVQPGGSLKLSCAASGFTF
NSYAMNWVRQAPGKGLEWVARIRSKYNNYATYYADSVKGRFTISRDDSKN
TAYLQMNNLKTEDTAVYYCVRHGNFGNSYVSWWAYWGQGTLVTVSSGGGG
SGGGGSGGGGSQTVVTQEPSLTVSPGGTVTLTCGSSTGAVTSGNYPNWVQ
QKPGQAPRGLIGGTKFLAPGTPARFSGSLLGGKAALTLSGVQPEDEAEYY
CVLWYSNRWVFGGGTKLTVLAAAEPKSSDKTHTCPPCPAPEAAGGPSVFL
FPPKPKDTLMISRTPEVTCVWDVSHEDPEVKFNWYVDGVEVHNAKTKPRE
EQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYDT
TPPVLDSDGSFFLYSDLTVDKSRWQQGNVFSCSVMHEALHNHYTQKSLSL
SPGK

SEQ ID NO:27 nucleic acid sequence encoding SEQ ID NO:26

atggggtcaaccgccatccttggcctcctcctggctgtcctgcagggagg
gcgcgcccaggatggcaacgaagaaatgggcggcattacccagaccccgt
ataaagtgagcattagcggcaccaccgtgattctgaccggaggcggcggt
tcaggcggaggtggctctggcggtggcggaagtggtggacctttgggtat
gcttagtcagagcgaggtgcagctggtcgagtctggaggaggattggtgc
agcctggagggtcattgaaactctcatgtgcagcctctggattcaccttc
aatagctacgccatgaactgggtccgccaggctccaggaaagggtttgga
atgggttgctcgcataagaagtaaatataataattatgcaacatattatg
ccgattcagtgaaaggcaggttcaccatctccagagatgattcaaaaaac
actgcctatctacaaatgaacaacttgaaaactgaggacactgccgtgta
ctactgtgtgagacatgggaacttcggtaatagctacgtttcctggtggg
cttactggggccaagggactctggtcaccgtctcctcaggtggtggtggt
tctggcggcggcggctccggtggtggtggttctcagactgttgtgactca
ggaaccttcactcaccgtatcacctggtggaacagtcacactcacttgtg
gctcctcgactggggctgttacatctggcaactacccaaactgggtccaa
caaaaaccaggtcaggcaccccgtggtctaataggtgggactaagttcct
cgcccccggtactcctgccagattctcaggctccctgcttggaggcaagg
ctgccctcaccctctcaggggtacagccagaggatgaggcagaatattac
tgtgttctatggtacagcaaccgctgggtgttcggtggaggaaccaaact
gactgtcctagcggccgcagagcccaaatcttctgacaaaactcacacat
gcccaccgtgcccagcacctgaagcagctgggggaccgtcagtcttcctc
ttccccccaaaacccaaggacaccctcatgatctcccggacccctgaggt
cacatgcgtggtggtggacgtgagccacgaagaccctgaggtcaagttca
actggtacgtggacggcgtggaggtgcataatgccaagacaaagccgcgg
gaggagcagtacaacagcacgtaccgtgtggtcagcgtcctcaccgtcct
gcaccaggactggctgaatggcaaggagtacaagtgcaaggtctccaaca
aagccctcccagcccccatcgagaaaaccatctccaaagccaaagggcag
ccccgagaaccacaggtgtacaccctgcccccatcccgggaggagatgac
caagaaccaggtcagcctgacctgcctggtcaaaggcttctatcccagcg
acatcgccgtggagtgggagagcaatgggcagccggagaacaactacgac
accacgcctcccgtgctggactccgacggctccttcttcctctatagcga
cctcaccgtggacaagagcaggtggcagcaggggaacgtcttctcatgct
ccgtgatgcatgaggctctgcacaaccactacacgcagaagagcctctcc
ctgtctccgggtaaa

SEQ ID NO:28 Amino acid sequence of the second polypeptide chain of CD3E(1-27)-FURINcsV2-aCD3-aHER2-mxb

MGSTAILGLLLAVLQGGRAQDGNEEMGGITQTPYKVSISGTTVILTGGGG
SGGGGSGGGGSGQSSRHRRALEVQLVESGGGLVQPGGSLKLSCAASGFTF
NSYAMNWVRQAPGKGLEWVARIRSKYNNYATYYADSVKGRFTISRDDSKN
TAYLQMNNLKTEDTAVYYCVRHGNFGNSYVSWWAYWGQGTLVTVSSGGGG
SGGGGSGGGGSQTVVTQEPSLTVSPGGTVTLTCGSSTGAVTSGNYPNWVQ
QKPGQAPRGLIGGTKFLAPGTPARFSGSLLGGKAALTLSGVQPEDEAEYY
CVLWYSNRWVFGGGTKLTVLAAAEPKSSDKTHTCPPCPAPEAAGGPSVFL
FPPKPKDTLMISRTPEVTCVWDVSHEDPEVKFNWYVDGVEVHNAKTKPRE
EQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYDT
TPPVLDSDGSFFLYSDLTVDKSRWQQGNVFSCSVMHEALHNHYTQKSLSL
SPGK

SEQ ID NO:29 Nucleic acid sequence encoding SEQ ID NO:28

atggggtcaaccgccatccttggcctcctcctggctgtcctgcagggagg
gcgcgcccaggatggcaacgaagaaatgggcggcattacccagaccccgt
ataaagtgagcattagcggcaccaccgtgattctgaccggaggcggcggt
tcaggcggaggtggctctggcggtggcggaagtggtcagagtagccgaca
cagacgtgcactagaggtgcagctggtcgagtctggaggaggattggtgc
agcctggagggtcattgaaactctcatgtgcagcctctggattcaccttc
aatagctacgccatgaactgggtccgccaggctccaggaaagggtttgga
atgggttgctcgcataagaagtaaatataataattatgcaacatattatg
ccgattcagtgaaaggcaggttcaccatctccagagatgattcaaaaaac
actgcctatctacaaatgaacaacttgaaaactgaggacactgccgtgta
ctactgtgtgagacatgggaacttcggtaatagctacgtttcctggtggg
cttactggggccaagggactctggtcaccgtctcctcaggtggtggtggt
tctggcggcggcggctccggtggtggtggttctcagactgttgtgactca
ggaaccttcactcaccgtatcacctggtggaacagtcacactcacttgtg
gctcctcgactggggctgttacatctggcaactacccaaactgggtccaa
caaaaaccaggtcaggcaccccgtggtctaataggtgggactaagttcct
cgcccccggtactcctgccagattctcaggctccctgcttggaggcaagg
ctgccctcaccctctcaggggtacagccagaggatgaggcagaatattac
tgtgttctatggtacagcaaccgctgggtgttcggtggaggaaccaaact
gactgtcctagcggccgcagagcccaaatcttctgacaaaactcacacat
gcccaccgtgcccagcacctgaagcagctgggggaccgtcagtcttcctc
ttccccccaaaacccaaggacaccctcatgatctcccggacccctgaggt
cacatgcgtggtggtggacgtgagccacgaagaccctgaggtcaagttca
actggtacgtggacggcgtggaggtgcataatgccaagacaaagccgcgg
gaggagcagtacaacagcacgtaccgtgtggtcagcgtcctcaccgtcct
gcaccaggactggctgaatggcaaggagtacaagtgcaaggtctccaaca
aagccctcccagcccccatcgagaaaaccatctccaaagccaaagggcag
ccccgagaaccacaggtgtacaccctgcccccatcccgggaggagatgac
caagaaccaggtcagcctgacctgcctggtcaaaggcttctatcccagcg
acatcgccgtggagtgggagagcaatgggcagccggagaacaactacgac
accacgcctcccgtgctggactccgacggctccttcttcctctatagcga
cctcaccgtggacaagagcaggtggcagcaggggaacgtcttctcatgct
ccgtgatgcatgaggctctgcacaaccactacacgcagaagagcctctcc
ctgtctccgggtaaa

SEQ ID NO:30 Amino acid sequence of the first polypeptide chain of aCD3-aHER2-Bi-Fc, CD3E(1-27)-aCD3-aHER2-Bi-Fc, CD3E(1-27)-MMP-2cs-aCD3-aHER2-Bi-Fc, and CD3E(1-27)-FURINcs-aCD3-aHER2-Bi-Fc (with signal sequence)

MGSTAILGLLLAVLQGGRAEVQLLEQSGAELVRPGALVKLSCKASGFKIK
DYFVNWVKQRPEQGLEWIGWIDPENDNSLYGPNFQDKASITADTSSNTGY
LQLSGLTSEDTAVYYCALYYGSRGDAMDYWGQGTTVTVSSGGGGSGGGGS
GGGGSELVMTQTPSSLSASLGDRVTISCRASQDISNYLNWYQQKPDGTVK
LLIYYTSRLHSGVPSRFSGSGSGTDYSLTISNLEQEDIATYFCQQGNTLP
LTFGAGTKLEIKSGGGGSEVQLVESGGGLVQPGGSLKLSCAASGFTFNSY
AMNWVRQAPGKGLEWVARIRSKYNNYATYYADSVKGRFTISRDDSKNTAY
LQMNNLKTEDTAVYYCVRHGNFGNSYVSWWAYWGQGTLVTVSSGGGGSGG
GGSGGGGSQTWTQEPSLTVSPGGTVTLTCGSSTGAVTSGNYPNWVQQKPG
QAPRGLIGGTKFLAPGTPARFSGSLLGGKAALTLSGVQPEDEAEYYCVLW
YSNRWVFGGGTKLTVLAAAEPKSSDKTHTCPPCPAPEAAGGPSVFLFPPK
PKDTLMISRTPEVTCVVVDVSHEDPEVKFNWYVDGVEVHNAKTKPREEQY
NSTYRWSVLTVLHQDWLNGKEYKCKVSNKALPAPIEKTISKAKGQPREPQ
VYTLPPSRKEMTKNQVSLTCLVKGFYPSDIAVEWESNGQPENNYKTTPPV
LKSDGSFFLYSKLTVDKSRWQQGNVFSCSVMHEALHNHYTQKSLSLSPGK
AAAHHHHHH

SEQ ID NO:31 Nucleic acid sequence encoding SEQ ID NO:30

atggggtcaaccgccatccttggcctcctcctggctgtcctgcagggagg
gcgcgccgaggtgcagctgctcgagcagtctggagctgagcttgtgaggc
caggggccttagtcaagttgtcctgcaaagcttctggcttcaaaattaaa
gactactttgtgaactgggtgaagcagaggcctgaacagggcctggagtg
gattggatggattgatcctgagaatgataatagtttatatggcccgaact
tccaggacaaggccagtatcacagcagacacatcctccaacacaggctac
ctgcagctcagcggcctgacatctgaggacactgccgtctattactgtgc
tctttattacggaagtaggggggatgctatggactactggggccaaggga
ccacggtcaccgtctcctcaggtggtggtggttctggcggcggcggctcc
ggtggtggtggttctgagctcgtgatgacccagactccatcctccctgtc
tgcctctctgggagacagagtcaccatcagttgcagggcaagtcaggaca
ttagcaattatttaaactggtatcagcagaaaccagatggaactgttaaa
ctcctgatctactacacatcaagattacactcaggagtcccatcaaggtt
cagtggcagtgggtctggaacagattattctctcaccattagcaacctgg
agcaagaagatattgccacttacttttgccaacagggtaatacgcttccg
ctcacgttcggtgctgggaccaagcttgagatcaaatccggaggtggtgg
atccgaggtgcagctggtcgagtctggaggaggattggtgcagcctggag
ggtcattgaaactctcatgtgcagcctctggattcaccttcaatagctac
gccatgaactgggtccgccaggctccaggaaagggtttggaatgggttgc
tcgcataagaagtaaatataataattatgcaacatattatgccgattcag
tgaaaggcaggttcaccatctccagagatgattcaaaaaacactgcctat
ctacaaatgaacaacttgaaaactgaggacactgccgtgtactactgtgt
gagacatgggaacttcggtaatagctacgtttcctggtgggcttactggg
gccaagggactctggtcaccgtctcctcaggtggtggtggttctggcggc
ggcggctccggtggtggtggttctcagactgttgtgactcaggaaccttc
actcaccgtatcacctggtggaacagtcacactcacttgtggctcctcga
ctggggctgttacatctggcaactacccaaactgggtccaacaaaaacca
ggtcaggcaccccgtggtctaataggtgggactaagttcctcgcccccgg
tactcctgccagattctcaggctccctgcttggaggcaaggctgccctca
ccctctcaggggtacagccagaggatgaggcagaatattactgtgttcta
tggtacagcaaccgctgggtgttcggtggaggaaccaaactgactgtcct
agcggccgcagagcccaaatcttctgacaaaactcacacatgccccccgt
gcccagcacctgaagcagctgggggaccgtcagtcttcctcttcccccca
aaacccaaggacaccctcatgatctcccggacccctgaggtcacatgcgt
ggtggtggacgtgagccacgaagaccctgaggtcaagttcaactggtacg
tggacggcgtggaggtgcataatgccaagacaaagccgcgagaggagcag
tacaacagcacgtaccgtgtggtcagcgtcctcaccgtcctgcaccagga
ctggctgaatggcaaggagtacaagtgcaaggtctccaacaaagccctcc
cagcccccatcgagaaaaccatctccaaagccaaagggcagccccgagaa
ccacaggtgtacaccctgcccccatcccggaaggagatgaccaagaacca
ggtcagcctgacctgcctggtcaaaggcttctatcccagcgacatcgccg
tggagtgggagagcaatgggcagccggagaacaactacaagaccacgcct
cccgtgctgaagtccgacggctccttcttcctctatagcaagctcaccgt
ggacaagagcaggtggcagcaggggaacgtcttctcatgctccgtgatgc
atgaggctctgcacaaccactacacgcagaagagcctctccctgtctccg
ggtaaagctgcagcgcatcaccaccaccatcac

SEQ ID NO:32 Amino acid sequence of the second polypeptide chain of aCD3-aHER2-Bi-Fc (with signal sequence)

MGSTAILALLLAVLQGVSAHMSSVSAQAAAEPKSSDKTHTCPPCPAPEAA
GGPSVFLFPPKPKDTLMISRTPEVTCWVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQ
PENNYDTTPPVLDSDGSFFLYSDLTVDKSRWQQGNVFSCSVMHEALHNHY
TQKSLSLSPGK

SEQ ID NO:33 Nucleic acid sequence encoding SEQ ID NO:32

atggggtcaaccgccatcctcgccctcctcctggctgttctccaaggagt
cagcgctcacatgtcttcggtaagtgcacaggcggccgcagagcccaaat
cttctgacaaaactcacacatgcccaccgtgcccagcacctgaagcagct
gggggaccgtcagtcttcctcttccccccaaaacccaaggacaccctcat
gatctcccggacccctgaggtcacatgcgtggtggtggacgtgagccacg
aagaccctgaggtcaagttcaactggtacgtggacggcgtggaggtgcat
aatgccaagacaaagccgcgggaggagcagtacaacagcacgtaccgtgt
ggtcagcgtcctcaccgtcctgcaccaggactggctgaatggcaaggagt
acaagtgcaaggtctccaacaaagccctcccagcccccatcgagaaaacc
atctccaaagccaaagggcagccccgagaaccacaggtgtacaccctgcc
cccatcccgggaggagatgaccaagaaccaggtcagcctgacctgcctgg
tcaaaggcttctatcccagcgacatcgccgtggagtgggagagcaatggg
cagccggagaacaactacgacaccacgcctcccgtgctggactccgacgg
ctccttcttcctctatagcgacctcaccgtggacaagagcaggtggcagc
aggggaacgtcttctcatgctccgtgatgcatgaggctctgcacaaccac
tacacgcagaagagcctctccctgtctccgggtaaa

SEQ ID NO:34 Amino acid sequence of the second polypeptide chain of CD3E(1-27)-aCD3-aHER2-Bi-Fc (with signal sequence)

MQSGTHWRVLGLCLLSVGVWGQDGNEEMGGITQTPYKVSISGTTVILTGG
GGSGGGGSGGGGSAAAEPKSSDKTHTCPPCPAPEAAGGPSVFLFPPKPKD
TLMISRTPEVTCVVVDVSHEDPEVKFNWYVDGVEVHNAKTKPREEQYNST
YRWSVLTVLHQDWLNGKEYKCKVSNKALPAPIEKTISKAKGQPREPQVYT
LPPSREEMTKNQVSLTCLVKGFYPSDIAVEWESNGQPENNYDTTPPVLDS
DGSFFLYSDLTVDKSRWQQGNVFSCSVMHEALHNHYTQKSLSLSPGK

SEQ ID NO:35 Nucleic acid sequence encoding SEQ ID NO:34

atgcagagcggcacccattggcgcgtgctgggcctgtgcctgctgagcgt
gggcgtgtggggccaggatggcaacgaagaaatgggcggcattacccaga
ccccgtataaagtgagcattagcggcaccaccgtgattctgaccggaggc
ggcggttcaggcggaggtggctctggcggtggcggaagtgcggccgcaga
gcccaaatcttctgacaaaactcacacatgcccaccgtgcccagcacctg
aagcagctgggggaccgtcagtcttcctcttccccccaaaacccaaggac
accctcatgatctcccggacccctgaggtcacatgcgtggtggtggacgt
gagccacgaagaccctgaggtcaagttcaactggtacgtggacggcgtgg
aggtgcataatgccaagacaaagccgcgggaggagcagtacaacagcacg
taccgtgtggtcagcgtcctcaccgtcctgcaccaggactggctgaatgg
caaggagtacaagtgcaaggtctccaacaaagccctcccagcccccatcg
agaaaaccatctccaaagccaaagggcagccccgagaaccacaggtgtac
accctgcccccatcccgggaggagatgaccaagaaccaggtcagcctgac
ctgcctggtcaaaggcttctatcccagcgacatcgccgtggagtgggaga
gcaatgggcagccggagaacaactacgacaccacgcctcccgtgctggac
tccgacggctccttcttcctctatagcgacctcaccgtggacaagagcag
gtggcagcaggggaacgtcttctcatgctccgtgatgcatgaggctctgc
acaaccactacacgcagaagagcctctccctgtctccgggtaaa

SEQ ID NO:36 Amino acid sequence of the second polypeptide chain of CD3E(1-27)-MMP-2cs-aCD3-aHER2-Bi-Fc (with signal sequence)

MQSGTHWRVLGLCLLSVGVWGQDGNEEMGGITQTPYKVSISGTTVILTGP
LGIAGQAAAEPKSSDKTHTCPPCPAPEAAGGPSVFLFPPKPKDTLMISRT
PEVTCVWDVSHEDPEVKFNWYVDGVEVHNAKTKPREEQYNSTYRVVSVLT
VLHQDWLNGKEYKCKVSNKALPAPIEKTISKAKGQPREPQVYTLPPSREE
MTKNQVSLTCLVKGFYPSDIAVEWESNGQPENNYDTTPPVLDSDGSFFLY
SDLTVDKSRWQQGNVFSCSVMHEALHNHYTQKSLSLSPGK

SEQ ID NO:37 Nucleic acid sequence encoding SEQ ID NO:36

atgcagagcggcacccattggcgcgtgctgggcctgtgcctgctgagcgt
gggcgtgtggggccaggatggcaacgaagaaatgggcggcattacccaga
ccccgtataaagtgagcattagcggcaccaccgtgattctgaccggaccg
ttgggtatcgctggccaggcggccgcagagcccaaatcttctgacaaaac
tcacacatgcccaccgtgcccagcacctgaagcagctgggggaccgtcag
tcttcctcttccccccaaaacccaaggacaccctcatgatctcccggacc
cctgaggtcacatgcgtggtggtggacgtgagccacgaagaccctgaggt
caagttcaactggtacgtggacggcgtggaggtgcataatgccaagacaa
agccgcgggaggagcagtacaacagcacgtaccgtgtggtcagcgtcctc
accgtcctgcaccaggactggctgaatggcaaggagtacaagtgcaaggt
ctccaacaaagccctcccagcccccatcgagaaaaccatctccaaagcca
aagggcagccccgagaaccacaggtgtacaccctgcccccatcccgggag
gagatgaccaagaaccaggtcagcctgacctgcctggtcaaaggcttcta
tcccagcgacatcgccgtggagtgggagagcaatgggcagccggagaaca
actacgacaccacgcctcccgtgctggactccgacggctccttcttcctc
tatagcgacctcaccgtggacaagagcaggtggcagcaggggaacgtctt
ctcatgctccgtgatgcatgaggctctgcacaaccactacacgcagaaga
gcctctccctgtctccgggtaaa

SEQ ID NO:38 Amino acid sequence of the second polypeptide chain of CD3E(1-27)-FURINcs-aCD3-aHER2-Bi-Fc (with signal sequence)

MQSGTHWRVLGLCLLSVGVWGQDGNEEMGGITQTPYKVSISGTTVILTRR
RRRAAAEPKSSDKTHTCPPCPAPEAAGGPSVFLFPPKPKDTLMISRTPEV
TCWVDVSHEDPEVKFNWYVDGVEVHNAKTKPREEQYNSTYRWSVLTVLHQ
DWLNGKEYKCKVSNKALPAPIEKTISKAKGQPREPQVYTLPPSREEMTKN
QVSLTCLVKGFYPSDIAVEWESNGQPENNYDTTPPVLDSDGSFFLYSDLT
VDKSRWQQGNVFSCSVMHEALHNHYTQKSLSLSPGK

SEQ ID NO:39 Nucleic acid sequence encoding SEQ ID NO:38

atgcagagcggcacccattggcgcgtgctgggcctgtgcctgctgagcgt
gggcgtgtggggccaggatggcaacgaagaaatgggcggcattacccaga
ccccgtataaagtgagcattagcggcaccaccgtgattctgacccggcga
agacgtcgcgcggccgcagagcccaaatcttctgacaaaactcacacatg
cccaccgtgcccagcacctgaagcagctgggggaccgtcagtcttcctct
tccccccaaaacccaaggacaccctcatgatctcccggacccctgaggtc
acatgcgtggtggtggacgtgagccacgaagaccctgaggtcaagttcaa
ctggtacgtggacggcgtggaggtgcataatgccaagacaaagccgcggg
aggagcagtacaacagcacgtaccgtgtggtcagcgtcctcaccgtcctg
caccaggactggctgaatggcaaggagtacaagtgcaaggtctccaacaa
agccctcccagcccccatcgagaaaaccatctccaaagccaaagggcagc
cccgagaaccacaggtgtacaccctgcccccatcccgggaggagatgacc
aagaaccaggtcagcctgacctgcctggtcaaaggcttctatcccagcga
catcgccgtggagtgggagagcaatgggcagccggagaacaactacgaca
ccacgcctcccgtgctggactccgacggctccttcttcctctatagcgac
ctcaccgtggacaagagcaggtggcagcaggggaacgtcttctcatgctc
cgtgatgcatgaggctctgcacaaccactacacgcagaagagcctctccc
tgtctccgggtaaa

SEQ ID NO:40 amino acid sequence of anti-CD3E VH region

EVQLVESGGGLVQPGGSLKLSCAASGFTFNKYAMNWVRQAPGKGLEWVAR
IRSKYNNYATYYADSVKDRFTISRDDSKNTAYLQMNNLKTEDTAVYYCVR
HGNFGNSYISYWAYWGQGTLVTVSS

SEQ ID NO:41 Nucleic acid sequence encoding SEQ ID NO:40

GAGGTGCAGCTGGTCGAGTCTGGAGGAGGATTGGTGCAGCCTGGAGGGTC
ATTGAAACTCTCATGTGCAGCCTCTGGATTCACCTTCAATAAGTACGCCA
TGAACTGGGTCCGCCAGGCTCCAGGAAAGGGTTTGGAATGGGTTGCTCGC
ATAAGAAGTAAATATAATAATTATGCAACATATTATGCCGATTCAGTGAA
AGACAGGTTCACCATCTCCAGAGATGATTCAAAAAACACTGCCTATCTAC
AAATGAACAACTTGAAAACTGAGGACACTGCCGTGTACTACTGTGTGAGA
CATGGGAACTTCGGTAATAGCTACATATCCTACTGGGCTTACTGGGGCCA
AGGGACTCTGGTCACCGTCTCCTCA

SEQ ID NO:42 AMINO ACID SEQUENCE OF HEAVY CHAIN CDR1 OF SEQ ID NO:40

KYAMN

SEQ ID NO:43 AMINO ACID SEQUENCE OF HEAVY CHAIN CDR2 OF SEQ ID NO:40 RIRSKYNNYATYYADSVKD

SEQ ID NO:44 AMINO ACID SEQUENCE OF HEAVY CHAIN CDR3 OF SEQ ID NO:40

HGNFGNSYISYWAY

SEQ ID NO:45 AMINO ACID SEQUENCE OF anti-CD3E VL region

QTWTQEPSLTVSPGGTVTLTCGSSTGAVTSGNYPNWVQQKPGQAPRGLIG
GTKFLAPGTPARFSGSLLGGKAALTLSGVQPEDEAEYYCVLWYSNRWVFG
GGTKLTVL

SEQ ID NO:46 NUCLEIC ACID SEQUENCE ENCODING SEQ ID NO:45

CAGACTGTTGTGACTCAGGAACCTTCACTCACCGTATCACCTGGTGGAAC
AGTCACACTCACTTGTGGCTCCTCGACTGGGGCTGTTACATCTGGCAACT
ACCCAAACTGGGTCCAACAAAAACCAGGTCAGGCACCCCGTGGTCTAATA
GGTGGGACTAAGTTCCTCGCCCCCGGTACTCCTGCCAGATTCTCAGGCTC
CCTGCTTGGAGGCAAGGCTGCCCTCACCCTCTCAGGGGTACAGCCAGAGG
ATGAGGCAGAATATTACTGTGTTCTATGGTACAGCAACCGCTGGGTGTTC
GGTGGAGGAACCAAACTGACTGTCCTA

SEQ ID NO:47 AMINO ACID SEQUENCE OF LIGHT CHAIN CDR1 OF SEQ ID NO:45

GSSTGAVTSGNYPN

SEQ ID NO:48 AMINO ACID SEQUENCE OF LIGHT CHAIN CDR2 OF SEQ ID NO:45

GTKFLAP

SEQ ID NO:49 AMINO ACID SEQUENCE OF LIGHT CHAIN CDR3 OF SEQ ID NO:45

VLWYSNRWV

SEQ ID NO:50 Amino acid sequence of the mature human CD3E

QDGNEEMGG ITQTPYKVSI SGTTVILTCP QYPGSEILWQ
HNDKNIGGDE DDKNIGSDED HLSLKEFSEL EQSGYYVCYP RGSKPEDANF YLYLRARVCE
NCMEMDVMSV ATIVIVDICI TGGLLLLVYY WSKNRKAKAK PVTRGAGAGG RQRGQNKERP
PPVPNPDYEP IRKGQRDLYS GLNQRRI

SEQ ID NO:51 Amino acid sequence of the mature CD3E of cynomolgus monkey

QDGNEEMGS ITQTPYQVSI SGTTVILTCS QHLGSEAQWQ
HNGKNKGDSG DQLFLPEFSE MEQSGYYVCY PRGSNPEDAS HHLYLKARVC ENCMEMDVMA
VATIVIVDIC ITLGLLLLVY YWSKNRKAKA KPVTRGAGAG GRQRGQNKER PPPVPNPDYE
PIRKGQQDLY SGLNQRRI

SEQ ID NO:52 Amino acid sequence of the extracellular domain of human CD3E

QDGNEEMGG ITQTPYKVSI SGTTVILTCP QYPGSEILWQ
HNDKNIGGDE DDKNIGSDED HLSLKEFSEL EQSGYYVCYP RGSKPEDANF YLYLRARVCE
NCMEMDVMS

SEQ ID NO:53 Amino acids 1-27 of human CD3E

QDGNEEMGG ITQTPYKVSI SGTTVILT

SEQ ID NO:54 Peptide sequence from human CD3E

Gln-Asp-Gly-Asn-Glu

SEQ ID NO:55 Amino acid sequence of a meprin α or β cleavage site

APMAEGGG

SEQ ID NO:56 Amino acid sequence of a meprin α or β cleavage site

EAQGDKII

SEQ ID NO:57 Amino acid sequence of a meprin α or β cleavage site

LAFSDAGP

SEQ ID NO:58 Amino acid sequence of a meprin α or β cleavage site

YVADAPK

SEQ ID NO:59 Amino acid sequence of a u-PA cleavage site

SGRSA

SEQ ID NO:60 Amino acid sequence of a u-PA cleavage site

GSGRSA

SEQ ID NO:61 Amino acid sequence of a u-PA cleavage site

SGKSA

SEQ ID NO:62 Amino acid sequence of a u-PA cleavage site

SGRSS

SEQ ID NO:63 Amino acid sequence of a u-PA cleavage site

SGRRA

SEQ ID NO:64 Amino acid sequence of a u-PA cleavage site

SGRNA

SEQ ID NO:65 Amino acid sequence of a u-PA cleavage site

SGRKA

SEQ ID NO:66 Amino acid sequence of a tPA cleavage site

QRGRSA

SEQ ID NO:67 Amino acid sequence of a cathepsin B cleavage site

TQGAAA

SEQ ID NO:68 Amino acid sequence of a cathepsin B cleavage site

GAAAAA

SEQ ID NO:69 Amino acid sequence of a cathepsin B cleavage site

GAGAAG

SEQ ID NO:70 Amino acid sequence of a cathepsin B cleavage site

AAAAAG

SEQ ID NO:71 Amino acid sequence of a cathepsin B cleavage site

LCGAAI

SEQ ID NO:72 Amino acid sequence of a cathepsin B cleavage site

FAQALG

SEQ ID NO:73 Amino acid sequence of a cathepsin B cleavage site

LAAANP

SEQ ID NO:74 Amino acid sequence of a cathepsin B cleavage site

LLQANP

SEQ ID NO:75 Amino acid sequence of a cathepsin B cleavage site

LAAANP

SEQ ID NO:76 Amino acid sequence of a cathepsin B cleavage site

LYGAQF

SEQ ID NO:77 Amino acid sequence of a cathepsin B cleavage site

LSQAQG

SEQ ID NO:78 Amino acid sequence of a cathepsin B cleavage site

ASAASG

SEQ ID NO:79 Amino acid sequence of a cathepsin B cleavage site

FLGASL

SEQ ID NO:80 Amino acid sequence of a cathepsin B cleavage site

AYGATG

SEQ ID NO:81 Amino acid sequence of a cathepsin B cleavage site

LAQATG

SEQ ID NO:82 Amino acid sequence of a furin cleavage site

RRRRRR

SEQ ID NO:83 Amino acid sequence of a fragment of human fibronectin

Val Pro Arg Asp Leu Glu Val Val Ala Ala Thr Pro Thr Ser Leu Leu
Ile Ser Trp Asp Ala Pro His His Gly Val Ala Tyr Tyr Arg Ile Thr
Tyr Gly Glu Thr Gly Gly Asn Ser Pro Val Gln Glu Phe Thr Val Pro
Gly Ser Lys Ser Thr Ala Thr Ile Ser Gly Leu Lys Pro Gly Val Asp
Tyr Thr Ile Asn Val Tyr Ala Val Leu Ala Tyr Pro Arg Gly Tyr Pro
Leu Ser Lys Pro Ile Ser Ile Asn Tyr Arg Thr

SEQ ID NO:84 Amino acid sequence of a human IgG1 Fc polypeptide chain

Glu Pro Lys Ser Cys Asp Lys Thr His Thr Cys Pro Pro Cys Pro Ala
Pro Glu Leu Leu Gly Gly Pro Ser Val Phe Leu Phe Pro Pro Lys Pro
Lys Asp Thr Leu Met Ile Ser Arg Thr Pro Glu Val Thr Cys Val Val
Val Asp Val Ser His Glu Asp Pro Glu Val Lys Phe Asn Trp Tyr Val
Asp Gly Val Glu Val His Asn Ala Lys Thr Lys Pro Arg Glu Glu Gln
Tyr Asn Ser Thr Tyr Arg Val Val Ser Val Leu Thr Val Leu His Gln
Asp Trp Leu Asn Gly Lys Glu Tyr Lys Cys Lys Val Ser Asn Lys Ala
Leu Pro Ala Pro Ile Glu Lys Thr Ile Ser Lys Ala Lys Gly Gln Pro
Arg Glu Pro Gln Val Tyr Thr Leu Pro Pro Ser Arg Glu Glu Met Thr
Lys Asn Gln Val Ser Leu Thr Cys Leu Val Lys Gly Phe Tyr Pro Ser
Asp Ile Ala Val Glu Trp Glu Ser Asn Gly Gln Pro Glu Asn Asn Tyr
Lys Thr Thr Pro Pro Val Leu Asp Ser Asp Gly Ser Phe Phe Leu Tyr
Ser Lys Leu Thr Val Asp Lys Ser Arg Trp Gln Gln Gly Asn Val Phe
Ser Cys Ser Val Met His Glu Ala Leu His Asn His Tyr Thr Gln Lys
Ser Leu Ser Leu Ser Pro Gly Lys

SEQ ID NO:85 Amino acid sequence of a human IgG2 Fc polypeptide chain

Glu Arg Lys Cys Cys Val Glu Cys Pro Pro Cys Pro Ala Pro Pro Val
Ala Gly Pro Ser Val Phe Leu Phe Pro Pro Lys Pro Lys Asp Thr Leu
Met Ile Ser Arg Thr Pro Glu Val Thr Cys Val Val Val Asp Val Ser
His Glu Asp Pro Glu Val Gln Phe Asn Trp Tyr Val Asp Gly Met Glu
Val His Asn Ala Lys Thr Lys Pro Arg Glu Glu Gln Phe Asn Ser Thr
Phe Arg Val Val Ser Val Leu Thr Val Val His Gln Asp Trp Leu Asn
Gly Lys Glu Tyr Lys Cys Lys Val Ser Asn Lys Gly Leu Pro Ala Pro
Ile Glu Lys Thr Ile Ser Lys Thr Lys Gly Gln Pro Arg Glu Pro Gln
Val Tyr Thr Leu Pro Pro Ser Arg Glu Glu Met Thr Lys Asn Gln Val
Ser Leu Thr Cys Leu Val Lys Gly Phe Tyr Pro Ser Asp Ile Ala Val
Glu Trp Glu Ser Asn Gly Gln Pro Glu Asn Asn Tyr Lys Thr Thr Pro
Pro Met Leu Asp Ser Asp Gly Ser Phe Phe Leu Tyr Ser Lys Leu Thr
Val Asp Lys Ser Arg Trp Gln Gln Gly Asn Val Phe Ser Cys Ser Val
Met His Glu Ala Leu His Asn His Tyr Thr Gln Lys Ser Leu Ser Leu
Ser Pro Gly Lys

SEQ ID NO:86 Amino acid sequence of a human IgG3 Fc polypeptide chain

Glu Leu Lys Thr Pro Leu Gly Asp Thr Thr His Thr Cys Pro Arg Cys
Pro Glu Pro Lys Ser Cys Asp Thr Pro Pro Pro Cys Pro Arg Cys Pro
Glu Pro Lys Ser Cys Asp Thr Pro Pro Pro Cys Pro Arg Cys Pro Glu
Pro Lys Ser Cys Asp Thr Pro Pro Pro Cys Pro Arg Cys Pro Ala Pro
Glu Leu Leu Gly Gly Pro Ser Val Phe Leu Phe Pro Pro Lys Pro Lys
Asp Thr Leu Met Ile Ser Arg Thr Pro Glu Val Thr Cys Val Val Val
Asp Val Ser His Glu Asp Pro Glu Val Gln Phe Lys Trp Tyr Val Asp
Gly Val Glu Val His Asn Ala Lys Thr Lys Pro Arg Glu Glu Gln Tyr
Asn Ser Thr Phe Arg Val Val Ser Val Leu Thr Val Leu His Gln Asp
Trp Leu Asn Gly Lys Glu Tyr Lys Cys Lys Val Ser Asn Lys Ala Leu
Pro Ala Pro Ile Glu Lys Thr Ile Ser Lys Thr Lys Gly Gln Pro Arg
Glu Pro Gln Val Tyr Thr Leu Pro Pro Ser Arg Glu Glu Met Thr Lys
Asn Gln Val Ser Leu Thr Cys Leu Val Lys Gly Phe Tyr Pro Ser Asp
Ile Ala Val Glu Trp Glu Ser Ser Gly Gln Pro Glu Asn Asn Tyr Asn
Thr Thr Pro Pro Met Leu Asp Ser Asp Gly Ser Phe Phe Leu Tyr Ser
Lys Leu Thr Val Asp Lys Ser Arg Trp Gln Gln Gly Asn Ile Phe Ser
Cys Ser Val Met His Glu Ala Leu His Asn Arg Phe Thr Gln Lys Ser
Leu Ser Leu Ser Pro Gly Lys

SEQ ID NO:87 Amino acid sequence of a human IgG4 Fc polypeptide chain

Glu Ser Lys Tyr Gly Pro Pro Cys Pro Ser Cys Pro Ala Pro Glu Phe
Leu Gly Gly Pro Ser Val Phe Leu Phe Pro Pro Lys Pro Lys Asp Thr
Leu Met Ile Ser Arg Thr Pro Glu Val Thr Cys Val Val Val Asp Val
Ser Gln Glu Asp Pro Glu Val Gln Phe Asn Trp Tyr Val Asp Gly Val
Glu Val His Asn Ala Lys Thr Lys Pro Arg Glu Glu Gln Phe Asn Ser
Thr Tyr Arg Val Val Ser Val Leu Thr Val Leu His Gln Asp Trp Leu
Asn Gly Lys Glu Tyr Lys Cys Lys Val Ser Asn Lys Gly Leu Pro Ser
Ser Ile Glu Lys Thr Ile Ser Lys Ala Lys Gly Gln Pro Arg Glu Pro
Gln Val Tyr Thr Leu Pro Pro Ser Gln Glu Glu Met Thr Lys Asn Gln
Val Ser Leu Thr Cys Leu Val Lys Gly Phe Tyr Pro Ser Asp Ile Ala
Val Glu Trp Glu Ser Asn Gly Gln Pro Glu Asn Asn Tyr Lys Thr Thr
Pro Pro Val Leu Asp Ser Asp Gly Ser Phe Phe Leu Tyr Ser Arg Leu
Thr Val Asp Lys Ser Arg Trp Gln Glu Gly Asn Val Phe Ser Cys Ser
Val Met His Glu Ala Leu His Asn His Tyr Thr Gln Lys Ser Leu Ser
Leu Ser Leu Gly Lys

SEQ ID NO:88 Amino acid sequence of a linker

(GGGGS) n

(where n is any integer from 1 to 10)

SEQ ID NO:89 Amino acid sequence of a linker

TVAAP

SEQ ID NO:90 Amino acid sequence of a linker

ASTKGP

SEQ ID NO:91 Amino acid sequence of a linker

GGGGSAAA

SEQ ID NO:92 Amino acid sequence of a linker

GGGGSGGGGSGGGGS

SEQ ID NO:93 Amino acid sequence of a second polypeptide of aCD3-aHER2-Xbody (not including signal sequence)

Glu Val Gln Leu Val Glu Ser Gly Gly Gly Leu Val Gln Pro Gly Gly
1 5 10 15
Ser Leu Lys Leu Ser Cys Ala Ala Ser Gly Phe Thr Phe Asn Ser Tyr
20 25 30
Ala Met Asn Trp Val Arg Gln Ala Pro Gly Lys Gly Leu Glu Trp Val
35 40 45
Ala Arg Ile Arg Ser Lys Tyr Asn Asn Tyr Ala Thr Tyr Tyr Ala Asp
50 55 60
Ser Val Lys Gly Arg Phe Thr Ile Ser Arg Asp Asp Ser Lys Asn Thr
65 70 75 80
Ala Tyr Leu Gln Met Asn Asn Leu Lys Thr Glu Asp Thr Ala Val Tyr
85 90 95
Tyr Cys Val Arg His Gly Asn Phe Gly Asn Ser Tyr Val Ser Trp Trp
100 105 110
Ala Tyr Trp Gly Gln Gly Thr Leu Val Thr Val Ser Ser Gly Gly Gly
115 120 125
Gly Ser Gly Gly Gly Gly Ser Glu Leu Val Met Thr Gln Thr Pro Ser
130 135 140
Ser Leu Ser Ala Ser Leu Gly Asp Arg Val Thr Ile Ser Cys Arg Ala
145 150 155 160
Ser Gln Asp Ile Ser Asn Tyr Leu Asn Trp Tyr Gln Gln Lys Pro Asp
165 170 175
Gly Thr Val Lys Leu Leu Ile Tyr Tyr Thr Ser Arg Leu His Ser Gly
180 185 190
Val Pro Ser Arg Phe Ser Gly Ser Gly Ser Gly Thr Asp Tyr Ser Leu
195 200 205
Thr Ile Ser Asn Leu Glu Gln Glu Asp Ile Ala Thr Tyr Phe Cys Gln
210 215 220
Gln Gly Asn Thr Leu Pro Leu Thr Phe Gly Ala Gly Thr Lys Leu Glu
225 230 235 240
Ile Lys Ala Ser Thr Lys Gly Pro Ser Val Phe Pro Leu Ala Pro Cys
245 250 255
Ser Arg Ser Thr Ser Glu Ser Thr Ala Ala Leu Gly Cys Leu Val Lys
260 265 270
Asp Tyr Phe Pro Glu Pro Val Thr Val Ser Trp Asn Ser Gly Ala Leu
275 280 285
Thr Ser Gly Val His Thr Phe Pro Ala Val Leu Gln Ser Ser Gly Leu
290 295 300
Tyr Ser Leu Ser Ser Val Val Thr Val Pro Ser Ser Asn Phe Gly Thr
305 310 315 320
Gln Thr Tyr Thr Cys Asn Val Asp His Lys Pro Ser Asn Thr Lys Val
325 330 335
Asp Lys Thr Val Gly Gly Gly Gly Ser Ala Ala Ala Glu Pro Lys Ser
340 345 350
Ser Asp Lys Thr His Thr Cys Pro Pro Cys Pro Ala Pro Glu Ala Ala
355 360 365
Gly Gly Pro Ser Val Phe Leu Phe Pro Pro Lys Pro Lys Asp Thr Leu
370 375 380
Met Ile Ser Arg Thr Pro Glu Val Thr Cys Val Val Val Asp Val Ser
385 390 395 400
His Glu Asp Pro Glu Val Lys Phe Asn Trp Tyr Val Asp Gly Val Glu
405 410 415
Val His Asn Ala Lys Thr Lys Pro Arg Glu Glu Gln Tyr Asn Ser Thr
420 425 430
Tyr Arg Val Val Ser Val Leu Thr Val Leu His Gln Asp Trp Leu Asn
435 440 445
Gly Lys Glu Tyr Lys Cys Lys Val Ser Asn Lys Ala Leu Pro Ala Pro
450 455 460
Ile Glu Lys Thr Ile Ser Lys Ala Lys Gly Gln Pro Arg Glu Pro Gln
465 470 475 480
Val Tyr Thr Leu Pro Pro Ser Arg Glu Glu Met Thr Lys Asn Gln Val
485 490 495
Ser Leu Thr Cys Leu Val Lys Gly Phe Tyr Pro Ser Asp Ile Ala Val
500 505 510
Glu Trp Glu Ser Asn Gly Gln Pro Glu Asn Asn Tyr Asp Thr Thr Pro
515 520 525
Pro Val Leu Asp Ser Asp Gly Ser Phe Phe Leu Tyr Ser Asp Leu Thr
530 535 540
Val Asp Lys Ser Arg Trp Gln Gln Gly Asn Val Phe Ser Cys Ser Val
545 550 555 560
Met His Glu Ala Leu His Asn His Tyr Thr Gln Lys Ser Leu Ser Leu
565 570 575
Ser Pro Gly Lys
580

SEQ ID NO:94 Amino acid sequence of a second polypeptide chain of aCD3-aHER2-mxb

EVQLVESGGGLVQPGGSLKLSCAASGFTFNSYAMNWVRQAPGKGLEWVAR
IRSKYNNYATYYADSVKGRFTISRDDSKNTAYLQMNNLKTEDTAVYYCVR
HGNFGNSYVSWWAYWGQGTLVTVSSGGGGSGGGGSGGGGSQTVVTQEPSL
TVSPGGTVTLTCGSSTGAVTSGNYPNWVQQKPGQAPRGLIGGTKFLAPGT
PARFSGSLLGGKAALTLSGVQPEDEAEYYCVLWYSNRWVFGGGTKLTVLA
AAEPKSSDKTHTCPPCPAPEAAGGPSVFLFPPKPKDTLMISRTPEVTCVW
DVSHEDPEVKFNWYVDGVEVHNAKTKPREEQYNSTYRVVSVLTVLHQDWL
NGKEYKCKVSNKALPAPIEKTISKAKGQPREPQVYTLPPSREEMTKNQVS
LTCLVKGFYPSDIAVEWESNGQPENNYDTTPPVLDSDGSFFLYSDLTVDK
SRWQQGNVFSCSVMHEALHNHYTQKSLSLSPGK

SEQ ID NO:95

AANLRN

SEQ ID NO:96

AQAYVK

SEQ ID NO:97

AANYMR

SEQ ID NO:98

AAALTR

SEQ ID NO:99

AQNLMR

SEQ ID NO:100

AANYTK

SEQ ID NO:101

GGCVFNMFNCGG

SEQ ID NO:102

GGCHLPFAVCGG

SEQ ID NO:103

GGCGHEYMWCGG

SEQ ID NO:104

GGCWPLQDYCGG

SEQ ID NO:105

GGCMQMNKWCGG

SEQ ID NO:106

GGCDGRTKYCGG

SEQ ID NO:107

GGCALYPTNCGG

SEQ ID NO:108

GGCGKHWHQCGG

SEQ ID NO:109

GGCHSFKHFCGG

SEQ ID NO:110

GGCQGMWTWCGG

SEQ ID NO:111

GGCAQQWHHEYCGG

SEQ ID NO:112

GGCERFHHACGG

1. A protein comprising
(a) one or more polypeptide chain(s) that bind to a target cell and that comprise a first pair of immunoglobulin heavy and light chain variable regions (VH1 and VL1) that bind to the target cell when part of an IgG or scFv antibody,
(b) one or more polypeptide chain(s) that bind to an effector cell and that comprise a second pair of immunoglobulin heavy and light chain variable regions (VH2 and VL2) that bind to the effector cell when part of an IgG or scFv antibody,
(c) a third polypeptide, and
(d) a linker comprising a protease cleavage site that links the third polypeptide of (c) to the remainder of the protein,
wherein a first polypeptide chain of the protein comprises an amino acid sequence having the formula: VH1-L1-VL1-L2-VH2-L3-VL2-X1, wherein L1, L2 and L3 are linkers, L3 can be present or absent, and X1 is a half-life-extending moiety,
wherein a second polypeptide chain of the protein comprises an amino acid sequence having the formula: Y-L4-X2, wherein Y is the polypeptide of (c), L4 is the linker comprising the protease cleavage site of (d), and X2 is a half-life-extending moiety, and
wherein the protein binds to either a target cell more effectively or to an effector cell more effectively when the protease cleavage site is essentially completely cleaved as compared to binding observed when the protease cleavage site is uncleaved.

2. The protein of claim 1, wherein the third polypeptide of (c) inhibits the binding of the protein to the effector cell.

3. A protein comprising
(a) one or more polypeptide chain(s) that bind to a target cell and that comprise a first pair of immunoglobulin heavy and light chain variable regions (VH1 and VL1) that bind to the target cell when part of an IgG or scFv antibody,
(b) one or more polypeptide chain(s) that bind to an effector cell and that comprise a second pair of immunoglobulin heavy and light chain variable regions (VH2 and VL2) that bind to the effector cell when part of an IgG or scFv antibody,
(c) a third polypeptide that inhibits the cytolytic activity of the protein in a cell cytolysis assay, and
(d) a linker comprising a protease cleavage site that links the third polypeptide of (c) to the remainder of the protein,
wherein a first polypeptide chain of the protein comprises an amino acid sequence having the formula: VH1-L1-VL1-L2-VH2-L3-VL2-X1, wherein L1, L2, and L3 are linkers, L3 can be present or absent, and X1 is a half-life-extending moiety,
wherein a second polypeptide chain of the protein comprises an amino acid sequence having the formula: Y-L4-X2, wherein Y is the polypeptide of (c), L4 is the linker comprising the protease cleavage site of (d), and X2 is a half-life-extending moiety, and
wherein the E_{c}50 of the protein in a cell cytolysis assay when the protease cleavage site is essentially completely cleaved is not more than a fifth of the E_{c}50 of the protein in the same assay when the protease cleavage site has not been cleaved.

4. The protein of claim 1, wherein the effector cell is a T cell.

5. The protein of claim 1, wherein the effector cell is an NK cell.

6. The protein of claim 4, wherein VH2 and VL2 bind to a polypeptide that is part of a TCR-CD3 complex when part of an IgG or ScFv antibody.

7. The protein of claim 6, wherein the polypeptide that is part of the TCR-CD3 complex is human CD3ε.

8. The protein of claim 7, wherein VH2 comprises a heavy chain CDR1, CDR2, and CDR3 comprising the amino acid sequences of SEQ ID NOs: 42, 43, and 44, respectively, and VL2 comprises a light chain CDR1, CDR2, and CDR3 comprising the amino acid sequences of SEQ ID NOs: 47, 48, and 49, respectively.

9. The protein of claim 8, wherein VH2 and VL2 comprise the amino acid sequences of SEQ ID NOs: 40 and 45, respectively.

10. The protein of claim 1, where the protease cleavable site can be cleaved by MMP-2, MMP-9, or MMP-11.

11. The protein of claim 10, wherein the protease cleavable site comprises an amino acid sequence selected from the group consisting of: GPLGIAGQ (SEQ ID NO:1), GGPLGMLSQS (SEQ ID NO:2), PLGLAG (SEQ ID NO:3), AANLRN (SEQ ID NO:95), AQAYVK (SEQ ID NO:96), AANYMR (SEQ ID NO: 97), AAALTR (SEQ ID NO:98), AQNLMR (SEQ ID NO:99), and AANYTK (SEQ ID NO:100).

12. The protein of claim 1, wherein X1 and X2 are Fc polypeptide chains.

13. The protein of claim 12, wherein the first polypeptide chain comprises the amino acid sequence of SEQ ID NO:30 and the second polypeptide chain comprises the amino acid sequence of SEQ ID NO:36 or SEQ ID NO:38.

14. The protein of claim 1, wherein the target cell is a cancer cell.

15. The protein of claim 14, wherein VH1 and VL1 bind to one of the following proteins when part of an IgG or ScFv antibody: epidermal growth factor receptor (EGFR), EGFRvlll, melanoma-associated chondroitin sulfate proteoglycan (MCSP), mesothelin (MSLN), folate receptor 1 (FOLR1), CD33, CDH19, or epidermal growth factor 2 (HER2).

16. A protein comprising one of the following pairs of polypeptide chains:
(a) (i) a first polypeptide chain comprising an amino acid sequence having the following formula: VH1-CH1-L1 -VH2-CH1, wherein VH1 and VH2 are immunoglobulin heavy chain variable regions, CH1 is a first heavy chain constant region, and L1 is a linker comprising a protease cleavable site, and
(ii) a second polypeptide chain comprising an amino acid sequence having the following formula: VL1-CL-L2-VL2-CL, wherein VL1 and VL2 are immunoglobulin light chain variable regions, CL is a light chain constant region, and L2 is a linker that does not contain a protease cleavage site, or
(b) (i) a first polypeptide chain comprising an amino acid sequence having the following formula: VH1-CH1-L1-VL2-CL, wherein VH1 is an immunoglobulin heavy chain variable region, VL2 is an immunoglobulin light chain variable region, CH1 is a first heavy chain constant region, CL is a light chain constant region, and L1 is a linker comprising a protease cleavage site, and
(ii) a second polypeptide chain comprising an amino acid sequence having the following formula: VL1-CL-L2-VH2-CH1, wherein VL is an immunoglobulin light chain variable regions, VH2 is an immunoglobulin heavy chain variable region, L2 is a linker that does not contain a protease cleavage site, and CH1 is a first heavy chain constant region, or
(c) (i) a first polypeptide chain comprising an amino acid sequence having the following formula: VL1-CL-L1-VL2-CL, wherein VL1 and V2 are immunoglobulin light chain variable regions, CL is a light chain constant region, and L1 is a linker comprising a protease cleavage site, and
(ii) a second polypeptide chain comprising an amino acid sequence having the following formula: VH1-CH1-L2-VH2-CH1, wherein VH1 and VH2 are heavy chain variable regions, L2 is a linker that does not contain a protease cleavage site, and CH1 is a first heavy chain constant region, or
(d) (i) a first polypeptide chain comprising an amino acid sequence having the following formula: VL1-CL-L1-VH2-CH1, wherein VH2 is an immunoglobulin heavy chain variable region, VL1 is an immunoglobulin light chain variable region, CH1 is a first heavy chain constant region, CL is a light chain constant region, and L1 is a protease-cleavable linker, and
(ii) a second polypeptide chain comprising an amino acid sequence having the following formula: VH1-CH1-L2-VL2-CL, wherein VL2 is an immunoglobulin light chain variable regions, VH1 is an immunoglobulin heavy chain variable region, L2 is a linker that does not contain a protease cleavage site, CH1 is a first heavy chain constant region, and CL is a light chain constant region,
wherein VL1 and VH1 bind to a target cell when part of an IgG or scFv antibody and VL2 and VH2 bind to an effector cell when part of an IgG or scFv antibody.

17. The protein of claim 16, wherein the effector cell is a T cell.

18. The protein of claim 17, wherein VH2 and VL2 bind to a protein that is part of a TCR-CD3 complex when part of an IgG or scFv antibody.

19. The protein of claim 17, wherein VH2 and VL2 bind to human CD3ε.

20. The protein of claim 19, wherein VH2 and VL2 comprise an immunoglobulin heavy chain CDR1, CDR2, and CDR3 comprising the amino acid sequence of SEQ ID NOs: 42, 43, and 44, respectively, and an immunoglobulin light chain CDR1, CDR2, and CDR3 comprising the amino acid sequence of SEQ ID NOs: 47, 48 and 49, respectively.

21. The protein of claim 20, wherein VH2 and VL2 comprise the amino acid sequences of SEQ ID NOs: 40 and 45, respectively.

22. The protein of claim 16, wherein the protease cleavage site comprises an amino acid sequence selected from the group consisting of: GPLGIAGQ (SEQ ID NO:1), GGPLGMLSQS (SEQ ID NO:2) PLGLAG (SEQ ID NO:3), AANLRN (SEQ ID NO:95), AQAYVK (SEQ ID NO:96), AANYMR (SEQ ID NO:97), AAALTR (SEQ ID NO:98), AQNLMR (SEQ ID NO:99), and AANYTK (SEQ ID NO:100).

23. The protein of claim 16, wherein the target cell is a cancer cell.

24. The protein of claim 23, wherein VH1 and VL1 bind to epidermal growth factor receptor (EGFR), EGFRvlll, melanoma-associated chondroitin sulfate proteoglycan (MCSP), mesothelin (MSLN), folate receptor 1 (FOLR1), CD33, CDH19, or epidermal growth factor 2 (HER2) when part of an igG or scFv antibody.

25. A nucleic acid comprising a nucleotide sequence encoding the protein of claim 1.

26. A vector containing the nucleic acid of claim 25.

27. A host cell containing the nucleic acid of claim 26.

28. A method of making a protein comprising
(a) one or more polypeptide chain(s) that bind to a target cell and that comprise a first pair of immunoglobulin heavy and light chain variable regions (VH1 and VL1) that bind to the target cell when part of an IgG or scFv antibody,
(b) one or more polypeptide chain(s) that bind to an effector cell and that comprise a second pair of immunoglobulin heavy and light chain variable regions (VH2 and VL2) that bind to the effector cell when part of an IgG or scFv antibody,
(c) a third polypeptide, and
(d) a linker comprising a protease cleavage site that links the third polypeptide of (c) to the remainder of the protein,
wherein a first polypeptide chain of the protein comprises an amino acid sequence having the formula: VH1-L1-VL1-L2-VH2-L3-VL2-X1, wherein L1, L2 and L3 are linkers, L3 can be present or absent, and X1 is a half-life-extending moiety,
wherein a second polypeptide chain of the protein comprises an amino acid sequence having the formula: Y-L4-X2, wherein Y is the polypeptide of (c), L4 is the linker comprising the protease cleavage site of (d), and X2 is a half-life-extending moiety, and
wherein the protein binds to either a target cell more effectively or to an effector cell more effectively when the protease cleavage site is essentially completely cleaved as compared to binding observed when the protease cleavage site is uncleaved comprising
culturing the host cell of claim 27 under conditions such that the protein is expressed, and
recovering the protein form the culture medium or the cell mass.

29. A method for treating a cancer patient comprising administering a therapeutically effective dose of the protein of claim 1 to the patient.

30. The method of claim 29, further comprising administering radiation, a chemotherapeutic agent, and/or a non-chemotherapeutic antineoplastic agent before, after, or concurrently with the protein.

31. The method of claim 29, wherein a cancer cell of the patient expresses a protease that can cleave the protease cleavage site.

32. A method for treating a patient suffering from an infection, a fibrotic disease, a neurodegenerative disease, or an autoimmune or inflammatory disease comprising administering a therapeutically effective dose of the protein of claim 1 to the patient.


SEQUENCE LISTING (118 sequences)

SEQ ID NO: 1 - protein, 8 aa, Artificial Sequence
  Amino acid sequence of an MMP2 cleavage site
GPLGIAGQ

SEQ ID NO: 2 - protein, 10 aa, Artificial Sequence
  Amino acid sequence of an MMP2 cleavage site
GGPLGMLSQS

SEQ ID NO: 3 - protein, 6 aa, Artificial Sequence
  Amino acid sequence of an MMP2 cleavage site
PLGLAG

SEQ ID NO: 4 - protein, 5 aa, Artificial Sequence
  Amino acid sequence of a furin cleavage site
RRRRR

SEQ ID NO: 5 - protein, 10 aa, Artificial Sequence
  Amino acid sequence of a furin cleavage site
GQSSRHRRAL

SEQ ID NO: 6 - protein, 609 aa, Artificial Sequence
  Amino acid sequence of the first polypeptide chain of a Xbody
MGSTAILGLLLAVLQGGRAEVQLLEQSGAELVRPGALVKLSCKASGFKIKDYFVNWVKQRPEQGLEWIGWIDPENDNSLYGPNFQDKASITADTSSNTGYLQLSGLTSEDTAVYYCALYYGSRGDAMDYWGQGTTVTVSSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGKAAAHHHHHH

SEQ ID NO: 7 - DNA, 1827 bp, Artificial Sequence
  Nucleic acid sequence encoding SEQ ID NO:6
atggggtcaaccgccatccttggcctcctcctggctgtcctgcagggagggcgcgccgaggtgcagctgctcgagcagtctggagctgagcttgtgaggccaggggccttagtcaagttgtcctgcaaagcttctggcttcaaaattaaagactactttgtgaactgggtgaagcagaggcctgaacagggcctggagtggattggatggattgatcctgagaatgataatagtttatatggcccgaacttccaggacaaggccagtatcacagcagacacatcctccaacacaggctacctgcagctcagcggcctgacatctgaggacactgccgtctattactgtgctctttattacggaagtaggggggatgctatggactactggggccaagggaccacggtcaccgtctcctcaggaggcggcggttcaggcggaggtggctctcagactgttgtgactcaggaaccttcactcaccgtatcacctggtggaacagtcacactcacttgtggctcctcgactggggctgttacatctggcaactacccaaactgggtccaacaaaaaccaggtcaggcaccccgtggtctaataggtgggactaagttcctcgcccccggtactcctgccagattctcaggctccctgcttggaggcaaggctgccctcaccctctcaggggtacagccagaggatgaggcagaatattactgtgttctatggtacagcaaccgctgggtgttcggtggaggaaccaaactgactgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcagcggccgcagagcccaaatcttctgacaaaactcacacatgccccccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgagaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagctgcagcgcatcaccaccaccatcac

SEQ ID NO: 8 - protein, 641 aa, Artificial Sequence
  Amino acid sequence of the second polypeptide chain of CD3Epsilon(1-27)-aCD3-aHER2-Xbody (including signal sequence)
MGSTAILGLLLAVLQGGRAQDGNEEMGGITQTPYKVSISGTTVILTGGGGSGGGGSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSELVMTQTPSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLEIKASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 9 - DNA, 1800 bp, Artificial Sequence
  Nucleic acid sequence encoding SEQ ID NO:8
atggggtcaaccgccatccttggcctcctcctggctgtcctgcagggagggcgcgcccaggatggcaacgaagaaatgggcggcattacccagaccccgtataaagtgagcattagcggcaccaccgtgattctgaccggaggcggcggttcaggcggaggtggctctggcggtggcggaagtgaggtgcagctggtcgagtctggaggcggattggtgcagcctggagggtcattgaaactctcatgtgcagcctctggattcaccttcaatagctacgccatgaactgggtccgccaggctccaggaaagggtttggaatgggttgctcgcataagaagtaaatataataattatgcaacatattatgccgattcagtgaaaggcaggttcaccatctccagagatgattcaaaaaacactgcctatctacaaatgaacaacttgaaaactgaggacactgccgtgtactactgtgtgagacatgggaacttcggtaatagctacgtttcctggtgggcttactggggccaagggactctggtcaccgtctcctcaggaggcggcggttcaggcggaggtggctctgagctcgtgatgacccagactccatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgctcacgttcggtgctgggaccaagcttgagatcaaagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggcggaggtggctctgcggccgcagagcccaaatcttctgacaaaactcacacatgcccaccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctc

SEQ ID NO: 10 - protein, 649 aa, Artificial Sequence
  Amino acid sequence of the second polypeptide chain of CD3Epsilon(1-27)-MMP-2csV1-aCD3-aHER2-Xbody (including signal sequence)
MGSTAILGLLLAVLQGGRAQDGNEEMGGITQTPYKVSISGTTVILTGGGGSGGGGSGGGGSGPLGIAGQEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSELVMTQTPSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLEIKASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 11 - DNA, 1947 bp, Artificial Sequence
  Nucleic acid sequence encoding SEQ ID NO:10
atggggtcaaccgccatccttggcctcctcctggctgtcctgcagggagggcgcgcccaggatggcaacgaagaaatgggcggcattacccagaccccgtataaagtgagcattagcggcaccaccgtgattctgaccggaggcggcggttcaggcggaggtggctctggcggtggcggaagtggaccgttgggtatcgctggccaggaggtgcagctggtcgagtctggaggaggattggtgcagcctggagggtcattgaaactctcatgtgcagcctctggattcaccttcaatagctacgccatgaactgggtccgccaggctccaggaaagggtttggaatgggttgctcgcataagaagtaaatataataattatgcaacatattatgccgattcagtgaaaggcaggttcaccatctccagagatgattcaaaaaacactgcctatctacaaatgaacaacttgaaaactgaggacactgccgtgtactactgtgtgagacatgggaacttcggtaatagctacgtttcctggtgggcttactggggccaagggactctggtcaccgtctcctcaggaggcggcggttcaggcggaggtggctctgagctcgtgatgacccagactccatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgctcacgttcggtgctgggaccaagcttgagatcaaagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggcggaggtggctctgcggccgcagagcccaaatcttctgacaaaactcacacatgcccaccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - protein, 651 aa, Artificial Sequence
  Amino acid sequence of the second polypeptide chain of CD3Epsilon(1-27)-MMP-2csV2-aCD3-aHER2-Xbody (including signal sequence)
MGSTAILGLLLAVLQGGRAQDGNEEMGGITQTPYKVSISGTTVILTGGGGSGGGGSGGGGSGGPLGMLSQSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSELVMTQTPSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLEIKASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 13 - DNA, 1953 bp, Artificial Sequence
  Nucleic acid sequence encoding SEQ ID NO:12
atggggtcaaccgccatccttggcctcctcctggctgtcctgcagggagggcgcgcccaggatggcaacgaagaaatgggcggcattacccagaccccgtataaagtgagcattagcggcaccaccgtgattctgaccggaggcggcggttcaggcggaggtggctctggcggtggcggaagtggtggacctttgggtatgcttagtcagagcgaggtgcagctggtcgagtctggaggaggattggtgcagcctggagggtcattgaaactctcatgtgcagcctctggattcaccttcaatagctacgccatgaactgggtccgccaggctccaggaaagggtttggaatgggttgctcgcataagaagtaaatataataattatgcaacatattatgccgattcagtgaaaggcaggttcaccatctccagagatgattcaaaaaacactgcctatctacaaatgaacaacttgaaaactgaggacactgccgtgtactactgtgtgagacatgggaacttcggtaatagctacgtttcctggtgggcttactggggccaagggactctggtcaccgtctcctcaggaggcggcggttcaggcggaggtggctctgagctcgtgatgacccagactccatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgctcacgttcggtgctgggaccaagcttgagatcaaagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggcggaggtggctctgcggccgcagagcccaaatcttctgacaaaactcacacatgcccaccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtaaa

SEQ ID NO: 14 - protein, 647 aa, Artificial Sequence
  Amino acid sequence of the second polypeptide chain of CD3Epsilon(1-27)-MMP-2csV3-aCD3-aHER2-Xbody (including signal sequence)
MGSTAILGLLLAVLQGGRAQDGNEEMGGITQTPYKVSISGTTVILTGGGGSGGGGSGGGGSPLGLAGEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSELVMTQTPSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLEIKASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 15 - DNA, 1941 bp, Artificial Sequence
  Nucleic acid sequence encoding SEQ ID NO:14
atggggtcaaccgccatccttggcctcctcctggctgtcctgcagggagggcgcgcccaggatggcaacgaagaaatgggcggcattacccagaccccgtataaagtgagcattagcggcaccaccgtgattctgaccggaggcggcggttcaggcggaggtggctctggcggtggcggaagtcctttgggtcttgccggagaggtgcagctggtcgagtctggaggcggattggtgcagcctggagggtcattgaaactctcatgtgcagcctctggattcaccttcaatagctacgccatgaactgggtccgccaggctccaggaaagggtttggaatgggttgctcgcataagaagtaaatataataattatgcaacatattatgccgattcagtgaaaggcaggttcaccatctccagagatgattcaaaaaacactgcctatctacaaatgaacaacttgaaaactgaggacactgccgtgtactactgtgtgagacatgggaacttcggtaatagctacgtttcctggtgggcttactggggccaagggactctggtcaccgtctcctcaggaggcggcggttcaggcggaggtggctctgagctcgtgatgacccagactccatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgctcacgttcggtgctgggaccaagcttgagatcaaagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggcggaggtggctctgcggccgcagagcccaaatcttctgacaaaactcacacatgcccaccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtaaa

SEQ ID NO: 16 - protein, 646 aa, Artificial Sequence
  Amino acid sequence of the second polypeptide chain of CD3Epsilon(1-27)-FURINcsV1-aCD3-aHER2-Xbody (including signal sequence)
MGSTAILGLLLAVLQGGRAQDGNEEMGGITQTPYKVSISGTTVILTGGGGSGGGGSGGGGSRRRRREVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSELVMTQTPSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLEIKASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 17 - DNA, 1938 bp, Artificial Sequence
  Nucleic acid sequence encoding SEQ ID NO:16
atggggtcaaccgccatccttggcctcctcctggctgtcctgcagggagggcgcgcccaggatggcaacgaagaaatgggcggcattacccagaccccgtataaagtgagcattagcggcaccaccgtgattctgaccggaggcggcggttcaggcggaggtggctctggcggtggcggaagtcggcgaagacgtcgcgaggtgcagctggtcgagtctggaggaggattggtgcagcctggagggtcattgaaactctcatgtgcagcctctggattcaccttcaatagctacgccatgaactgggtccgccaggctccaggaaagggtttggaatgggttgctcgcataagaagtaaatataataattatgcaacatattatgccgattcagtgaaaggcaggttcaccatctccagagatgattcaaaaaacactgcctatctacaaatgaacaacttgaaaactgaggacactgccgtgtactactgtgtgagacatgggaacttcggtaatagctacgtttcctggtgggcttactggggccaagggactctggtcaccgtctcctcaggaggcggcggttcaggcggaggtggctctgagctcgtgatgacccagactccatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgctcacgttcggtgctgggaccaagcttgagatcaaagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggcggaggtggctctgcggccgcagagcccaaatcttctgacaaaactcacacatgcccaccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtaaa

SEQ ID NO: 18 - protein, 651 aa, Artificial Sequence
  Amino acid sequence of the second polypeptide chain of CD3Epsilon(1-27)-FURINcsV2-aCD3-aHER2-Xbody (including signal sequence)
MGSTAIFGLLLAVLQGGRAQDGNEEMGGITQTPYKVSISGTTVILTGGGGSGGGGSGGGGSGQSSRHRRALEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSELVMTQTPSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLEIKASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 19 - DNA, 1953 bp, Artificial Sequence
  Nucleic acid sequence encoding SEQ ID NO:18
atggggtcaaccgccatctttggcctcctcctggctgtcctgcagggagggcgcgcccaggatggcaacgaagaaatgggcggcattacccagaccccgtataaagtgagcattagcggcaccaccgtgattctgaccggaggcggcggttcaggcggaggtggctctggcggtggcggaagtggtcagagtagccgacacagacgtgcactagaggtgcagctggtcgagtctggaggaggattggtgcagcctggagggtcattgaaactctcatgtgcagcctctggattcaccttcaatagctacgccatgaactgggtccgccaggctccaggaaagggtttggaatgggttgctcgcataagaagtaaatataataattatgcaacatattatgccgattcagtgaaaggcaggttcaccatctccagagatgattcaaaaaacactgcctatctacaaatgaacaacttgaaaactgaggacactgccgtgtactactgtgtgagacatgggaacttcggtaatagctacgtttcctggtgggcttactggggccaagggactctggtcaccgtctcctcaggaggcggcggttcaggcggaggtggctctgagctcgtgatgacccagactccatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgctcacgttcggtgctgggaccaagcttgagatcaaagctagcaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttggcggaggtggctctgcggccgcagagcccaaatcttctgacaaaactcacacatgcccaccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtaaa

SEQ ID NO: 20 - protein, 506 aa, Artificial Sequence
  Amino acid sequence of the first polypeptide chain of CD3Epsilon(1-27)-aCD3-aHER2-mxb, CD3Epsilon(1-27)-MMP-2csV1-aCD3-aHER2-mxb, CD3Epsilon(1-27)-MMP-2csV2-aCD3-aHER2-mxb, and
MGSTAILGLLLAVLQGGRAEVQLLEQSGAELVRPGALVKLSCKASGFKIKDYFVNWVKQRPEQGLEWIGWIDPENDNSLYGPNFQDKASITADTSSNTGYLQLSGLTSEDTAVYYCALYYGSRGDAMDYWGQGTTVTVSSGGGGSGGGGSGGGGSELVMTQTPSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLEIKAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGKAAAHHHHHH

SEQ ID NO: 21 - DNA, 1518 bp, Artificial Sequence
  Nucleic acid sequence encoding SEQ ID NO:20
atggggtcaaccgccatccttggcctcctcctggctgtcctgcagggagggcgcgccgaggtgcagctgctcgagcagtctggagctgagcttgtgaggccaggggccttagtcaagttgtcctgcaaagcttctggcttcaaaattaaagactactttgtgaactgggtgaagcagaggcctgaacagggcctggagtggattggatggattgatcctgagaatgataatagtttatatggcccgaacttccaggacaaggccagtatcacagcagacacatcctccaacacaggctacctgcagctcagcggcctgacatctgaggacactgccgtctattactgtgctctttattacggaagtaggggggatgctatggactactggggccaagggaccacggtcaccgtctcctcaggtggtggtggttctggcggcggcggctccggtggtggtggttctgagctcgtgatgacccagactccatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgctcacgttcggtgctgggaccaagcttgagatcaaagcggccgcagagcccaaatcttctgacaaaactcacacatgccccccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgagaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagctgcagcgcatcaccaccaccatcac

SEQ ID NO: 22 - protein, 545 aa, Artificial Sequence
  Amino acid sequence of the second polypeptide chain of CD3Epsilon(1-27)-aCD3-aHER2-mxb (including signal sequence)
MGSTAILGLLLAVLQGGRAQDGNEEMGGITQTPYKVSISGTTVILTGGGGSGGGGSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 23 - DNA, 1635 bp, Artificial Sequence
  Nucleic acid sequence encoding SEQ ID NO:22
atggggtcaaccgccatccttggcctcctcctggctgtcctgcagggagggcgcgcccaggatggcaacgaagaaatgggcggcattacccagaccccgtataaagtgagcattagcggcaccaccgtgattctgaccggaggcggcggttcaggcggaggtggctctggcggtggcggaagtgaggtgcagctggtcgagtctggaggaggattggtgcagcctggagggtcattgaaactctcatgtgcagcctctggattcaccttcaatagctacgccatgaactgggtccgccaggctccaggaaagggtttggaatgggttgctcgcataagaagtaaatataataattatgcaacatattatgccgattcagtgaaaggcaggttcaccatctccagagatgattcaaaaaacactgcctatctacaaatgaacaacttgaaaactgaggacactgccgtgtactactgtgtgagacatgggaacttcggtaatagctacgtttcctggtgggcttactggggccaagggactctggtcaccgtctcctcaggtggtggtggttctggcggcggcggctccggtggtggtggttctcagactgttgtgactcaggaaccttcactcaccgtatcacctggtggaacagtcacactcacttgtggctcctcgactggggctgttacatctggcaactacccaaactgggtccaacaaaaaccaggtcaggcaccccgtggtctaataggtgggactaagttcctcgcccccggtactcctgccagattctcaggctccctgcttggaggcaaggctgccctcaccctctcaggggtacagccagaggatgaggcagaatattactgtgttctatggtacagcaaccgctgggtgttcggtggaggaaccaaactgactgtcctagcggccgcagagcccaaatcttctgacaaaactcacacatgcccaccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtaaa

SEQ ID NO: 24 - protein, 553 aa, Artificial Sequence
  Amino acid sequence of the second polypeptide chain of CD3Epsilon(1-27)-MMP-2csV1-aCD3-aHER2-mxb (including signal sequence)
MGSTAILGLLLAVLQGGRAQDGNEEMGGITQTPYKVSISGTTVILTGGGGSGGGGSGGGGSGPLGIAGQEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 25 - DNA, 1659 bp, Artificial Sequence
  Nucleic acid sequence encoding SEQ ID NO:24
atggggtcaaccgccatccttggcctcctcctggctgtcctgcagggagggcgcgcccaggatggcaacgaagaaatgggcggcattacccagaccccgtataaagtgagcattagcggcaccaccgtgattctgaccggaggcggcggttcaggcggaggtggctctggcggtggcggaagtggaccgttgggtatcgctggccaggaggtgcagctggtcgagtctggaggaggattggtgcagcctggagggtcattgaaactctcatgtgcagcctctggattcaccttcaatagctacgccatgaactgggtccgccaggctccaggaaagggtttggaatgggttgctcgcataagaagtaaatataataattatgcaacatattatgccgattcagtgaaaggcaggttcaccatctccagagatgattcaaaaaacactgcctatctacaaatgaacaacttgaaaactgaggacactgccgtgtactactgtgtgagacatgggaacttcggtaatagctacgtttcctggtgggcttactggggccaagggactctggtcaccgtctcctcaggtggtggtggttctggcggcggcggctccggtggtggtggttctcagactgttgtgactcaggaaccttcactcaccgtatcacctggtggaacagtcacactcacttgtggctcctcgactggggctgttacatctggcaactacccaaactgggtccaacaaaaaccaggtcaggcaccccgtggtctaataggtgggactaagttcctcgcccccggtactcctgccagattctcaggctccctgcttggaggcaaggctgccctcaccctctcaggggtacagccagaggatgaggcagaatattactgtgttctatggtacagcaaccgctgggtgttcggtggaggaaccaaactgactgtcctagcggccgcagagcccaaatcttctgacaaaactcacacatgcccaccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtaaa

SEQ ID NO: 26 - protein, 555 aa, Artificial Sequence
  Amino acid sequence of the second polypeptide chain of CD3Epsilon(1-27)-MMP-2csV2-aCD3-aHER2-mxb (including signal sequence)
MGSTAILGLLLAVLQGGRAQDGNEEMGGITQTPYKVSISGTTVILTGGGGSGGGGSGGGGSGGPLGMLSQSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 27 - DNA, 1665 bp, Artificial Sequence
  Nucleic acid sequence encoding SEQ ID NO:26
atggggtcaaccgccatccttggcctcctcctggctgtcctgcagggagggcgcgcccaggatggcaacgaagaaatgggcggcattacccagaccccgtataaagtgagcattagcggcaccaccgtgattctgaccggaggcggcggttcaggcggaggtggctctggcggtggcggaagtggtggacctttgggtatgcttagtcagagcgaggtgcagctggtcgagtctggaggaggattggtgcagcctggagggtcattgaaactctcatgtgcagcctctggattcaccttcaatagctacgccatgaactgggtccgccaggctccaggaaagggtttggaatgggttgctcgcataagaagtaaatataataattatgcaacatattatgccgattcagtgaaaggcaggttcaccatctccagagatgattcaaaaaacactgcctatctacaaatgaacaacttgaaaactgaggacactgccgtgtactactgtgtgagacatgggaacttcggtaatagctacgtttcctggtgggcttactggggccaagggactctggtcaccgtctcctcaggtggtggtggttctggcggcggcggctccggtggtggtggttctcagactgttgtgactcaggaaccttcactcaccgtatcacctggtggaacagtcacactcacttgtggctcctcgactggggctgttacatctggcaactacccaaactgggtccaacaaaaaccaggtcaggcaccccgtggtctaataggtgggactaagttcctcgcccccggtactcctgccagattctcaggctccctgcttggaggcaaggctgccctcaccctctcaggggtacagccagaggatgaggcagaatattactgtgttctatggtacagcaaccgctgggtgttcggtggaggaaccaaactgactgtcctagcggccgcagagcccaaatcttctgacaaaactcacacatgcccaccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtaaa

SEQ ID NO: 28 - protein, 555 aa, Artificial Sequence
  Amino acid sequence of the second polypeptide chain of CD3Epsilon(1-27)-FURINcsV2-aCD3-aHER2-mxb (including signal sequence)
MGSTAILGLLLAVLQGGRAQDGNEEMGGITQTPYKVSISGTTVILTGGGGSGGGGSGGGGSGQSSRHRRALEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 29 - DNA, 1665 bp, Artificial Sequence
  Nucleic acid sequence encoding SEQ ID NO:28
atggggtcaaccgccatccttggcctcctcctggctgtcctgcagggagggcgcgcccaggatggcaacgaagaaatgggcggcattacccagaccccgtataaagtgagcattagcggcaccaccgtgattctgaccggaggcggcggttcaggcggaggtggctctggcggtggcggaagtggtcagagtagccgacacagacgtgcactagaggtgcagctggtcgagtctggaggaggattggtgcagcctggagggtcattgaaactctcatgtgcagcctctggattcaccttcaatagctacgccatgaactgggtccgccaggctccaggaaagggtttggaatgggttgctcgcataagaagtaaatataataattatgcaacatattatgccgattcagtgaaaggcaggttcaccatctccagagatgattcaaaaaacactgcctatctacaaatgaacaacttgaaaactgaggacactgccgtgtactactgtgtgagacatgggaacttcggtaatagctacgtttcctggtgggcttactggggccaagggactctggtcaccgtctcctcaggtggtggtggttctggcggcggcggctccggtggtggtggttctcagactgttgtgactcaggaaccttcactcaccgtatcacctggtggaacagtcacactcacttgtggctcctcgactggggctgttacatctggcaactacccaaactgggtccaacaaaaaccaggtcaggcaccccgtggtctaataggtgggactaagttcctcgcccccggtactcctgccagattctcaggctccctgcttggaggcaaggctgccctcaccctctcaggggtacagccagaggatgaggcagaatattactgtgttctatggtacagcaaccgctgggtgttcggtggaggaaccaaactgactgtcctagcggccgcagagcccaaatcttctgacaaaactcacacatgcccaccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtaaa

SEQ ID NO: 30 - protein, 761 aa, Artificial Sequence
  Amino acid sequence of the first polypeptide chain of aCD3-aHER2-Bi-Fc, CD3Epsilon(1-27)-aCD3-aHER2-Bi-Fc, CD3Epsilon(1-27)-MMP-2cs-aCD3-aHER2-Bi-Fc, and CD3Epsilon(1-27)-FURINcs-aCD3-aHER2-Bi-Fc (including signal
MGSTAILGLLLAVLQGGRAEVQLLEQSGAELVRPGALVKLSCKASGFKIKDYFVNWVKQRPEQGLEWIGWIDPENDNSLYGPNFQDKASITADTSSNTGYLQLSGLTSEDTAVYYCALYYGSRGDAMDYWGQGTTVTVSSGGGGSGGGGSGGGGSELVMTQTPSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGKAAAHHHHHH

SEQ ID NO: 31 - DNA, 2283 bp, Artificial Sequence
  Nucleic acid sequence encoding SEQ ID NO:30
atggggtcaaccgccatccttggcctcctcctggctgtcctgcagggagggcgcgccgaggtgcagctgctcgagcagtctggagctgagcttgtgaggccaggggccttagtcaagttgtcctgcaaagcttctggcttcaaaattaaagactactttgtgaactgggtgaagcagaggcctgaacagggcctggagtggattggatggattgatcctgagaatgataatagtttatatggcccgaacttccaggacaaggccagtatcacagcagacacatcctccaacacaggctacctgcagctcagcggcctgacatctgaggacactgccgtctattactgtgctctttattacggaagtaggggggatgctatggactactggggccaagggaccacggtcaccgtctcctcaggtggtggtggttctggcggcggcggctccggtggtggtggttctgagctcgtgatgacccagactccatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgctcacgttcggtgctgggaccaagcttgagatcaaatccggaggtggtggatccgaggtgcagctggtcgagtctggaggaggattggtgcagcctggagggtcattgaaactctcatgtgcagcctctggattcaccttcaatagctacgccatgaactgggtccgccaggctccaggaaagggtttggaatgggttgctcgcataagaagtaaatataataattatgcaacatattatgccgattcagtgaaaggcaggttcaccatctccagagatgattcaaaaaacactgcctatctacaaatgaacaacttgaaaactgaggacactgccgtgtactactgtgtgagacatgggaacttcggtaatagctacgtttcctggtgggcttactggggccaagggactctggtcaccgtctcctcaggtggtggtggttctggcggcggcggctccggtggtggtggttctcagactgttgtgactcaggaaccttcactcaccgtatcacctggtggaacagtcacactcacttgtggctcctcgactggggctgttacatctggcaactacccaaactgggtccaacaaaaaccaggtcaggcaccccgtggtctaataggtgggactaagttcctcgcccccggtactcctgccagattctcaggctccctgcttggaggcaaggctgccctcaccctctcaggggtacagccagaggatgaggcagaatattactgtgttctatggtacagcaaccgctgggtgttcggtggaggaaccaaactgactgtcctagcggccgcagagcccaaatcttctgacaaaactcacacatgccccccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgagaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaagctgcagcgcatcaccaccaccatcac

SEQ ID NO: 32 - protein, 262 aa, Artificial Sequence
  Amino acid sequence of the second polypeptide chain of aCD3-aHER2-Bi-Fc (including signal sequence)
MGSTAILALLLAVLQGVSAHMSSVSAQAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 33 - DNA, 786 bp, Artificial Sequence
  Nucleic acid sequence encoding SEQ ID NO:32
atggggtcaaccgccatcctcgccctcctcctggctgttctccaaggagtcagcgctcacatgtcttcggtaagtgcacaggcggccgcagagcccaaatcttctgacaaaactcacacatgcccaccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtaaa

SEQ ID NO: 34 - protein, 298 aa, Artificial Sequence
  Amino acid sequence of the second polypeptide chain of CD3Epsilon(1-27)-aCD3-aHER2-Bi-Fc (including signal sequence)
MQSGTHWRVLGLCLLSVGVWGQDGNEEMGGITQTPYKVSISGTTVILTGGGGSGGGGSGGGGSAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 35 - DNA, 894 bp, Artificial Sequence
  Nucleic acid sequence encoding SEQ ID NO:34
atgcagagcggcacccattggcgcgtgctgggcctgtgcctgctgagcgtgggcgtgtggggccaggatggcaacgaagaaatgggcggcattacccagaccccgtataaagtgagcattagcggcaccaccgtgattctgaccggaggcggcggttcaggcggaggtggctctggcggtggcggaagtgcggccgcagagcccaaatcttctgacaaaactcacacatgcccaccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtaaa

SEQ ID NO: 36 - protein, 291 aa, Artificial Sequence
  Amino acid sequence of the second polypeptide chain of CD3Epsilon(1-27)-MMP-2cs-aCD3-aHER2-Bi-Fc (including signal sequence)
MQSGTHWRVLGLCLLSVGVWGQDGNEEMGGITQTPYKVSISGTTVILTGPLGIAGQAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 37 - DNA, 873 bp, Artificial Sequence
  Nucleic acid sequence encoding SEQ ID NO:36
atgcagagcggcacccattggcgcgtgctgggcctgtgcctgctgagcgtgggcgtgtggggccaggatggcaacgaagaaatgggcggcattacccagaccccgtataaagtgagcattagcggcaccaccgtgattctgaccggaccgttgggtatcgctggccaggcggccgcagagcccaaatcttctgacaaaactcacacatgcccaccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtaaa

SEQ ID NO: 38 - protein, 288 aa, Artificial Sequence
  Amino acid sequence of the second polypeptide chain of CD3Epsilon(1-27)-FURINcs-aCD3-aHER2-Bi-Fc (including signal sequence)
MQSGTHWRVLGLCLLSVGVWGQDGNEEMGGITQTPYKVSISGTTVILTRRRRRAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 39 - DNA, 864 bp, Artificial Sequence
  Nucleic acid sequence encoding SEQ ID NO:38
atgcagagcggcacccattggcgcgtgctgggcctgtgcctgctgagcgtgggcgtgtggggccaggatggcaacgaagaaatgggcggcattacccagaccccgtataaagtgagcattagcggcaccaccgtgattctgacccggcgaagacgtcgcgcggccgcagagcccaaatcttctgacaaaactcacacatgcccaccgtgcccagcacctgaagcagctgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggtaaa

SEQ ID NO: 40 - protein, 125 aa, Artificial Sequence
  Amino acid sequence of an anti-CD3Epsilon VH region
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 41 - DNA, 375 bp, Artificial Sequence
  Nucleic acid sequence encoding SEQ ID NO:40
gaggtgcagctggtcgagtctggaggaggattggtgcagcctggagggtcattgaaactctcatgtgcagcctctggattcaccttcaataagtacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacaacttgaaaactgaggacactgccgtgtactactgtgtgagacatgggaacttcggtaatagctacatatcctactgggcttactggggccaagggactctggtcaccgtctcctca

SEQ ID NO: 42 - protein, 5 aa, Artificial Sequence
  Amino acid sequence of a heavy chain CDR1 of SEQ ID NO:40
KYAMN

SEQ ID NO: 43 - protein, 19 aa, Artificial Sequence
  Amino acid sequence of a heavy chain CDR2 of SEQ ID NO:40
RIRSKYNNYATYYADSVKD

SEQ ID NO: 44 - protein, 14 aa, Artificial Sequence
  Amino acid sequence of a heavy chain CDR3 of SEQ ID NO:40
HGNFGNSYISYWAY

SEQ ID NO: 45 - protein, 109 aa, Artificial Sequence
  Amino acid sequence of an anti-CD3Epsilon VL region
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 46 - DNA, 327 bp, Artificial Sequence
  Nucleic acid sequence encoding SEQ ID NO:45
cagactgttgtgactcaggaaccttcactcaccgtatcacctggtggaacagtcacactcacttgtggctcctcgactggggctgttacatctggcaactacccaaactgggtccaacaaaaaccaggtcaggcaccccgtggtctaataggtgggactaagttcctcgcccccggtactcctgccagattctcaggctccctgcttggaggcaaggctgccctcaccctctcaggggtacagccagaggatgaggcagaatattactgtgttctatggtacagcaaccgctgggtgttcggtggaggaaccaaactgactgtccta

SEQ ID NO: 47 - protein, 14 aa, Artificial Sequence
  Amino acid sequence of a light chain CDR1 of SEQ ID NO:45
GSSTGAVTSGNYPN

SEQ ID NO: 48 - protein, 7 aa, Artificial Sequence
  Amino acid sequence of a light chain CDR2 of SEQ ID NO:45
GTKFLAP

SEQ ID NO: 49 - protein, 9 aa, Artificial Sequence
  Amino acid sequence of a light chain CDR3 of SEQ ID NO:45
VLWYSNRWV

SEQ ID NO: 50 - protein, 186 aa, Artificial Sequence
  Mature amino acid sequence of human CD3Epsilon
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 51 - protein, 177 aa, Artificial Sequence
  Mature amino acid sequence of cynomolgus monkey CD3Epsilon
QDGNEEMGSITQTPYQVSISGTTVILTCSQHLGSEAQWQHNGKNKGDSGDQLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 52 - protein, 108 aa, Artificial Sequence
  Amino acid sequence of the extracellular domain of human CD3Epsilon
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMS

SEQ ID NO: 53 - protein, 27 aa, Artificial Sequence
  Amino acids 1-27 of mature human CD3Epsilon
QDGNEEMGGITQTPYKVSISGTTVILT

SEQ ID NO: 54 - protein, 5 aa, Artificial Sequence
  Peptide sequence from human CD3Epsilon
QDGNE

SEQ ID NO: 55 - protein, 8 aa, Artificial Sequence
  Amino acid sequence of a meprin Alpha or Beta cleavage site
APMAEGGG

SEQ ID NO: 56 - protein, 8 aa, Artificial Sequence
  Amino acid sequence of a meprin Alpha or Beta cleavage site
EAQGDKII

SEQ ID NO: 57 - protein, 8 aa, Artificial Sequence
  Amino acid sequence of a meprin Alpha or Beta cleavage site
LAFSDAGP

SEQ ID NO: 58 - protein, 7 aa, Artificial Sequence
  Amino acid sequence of a meprin Alpha or Beta cleavage site
YVADAPK

SEQ ID NO: 59 - protein, 5 aa, Artificial Sequence
  Amino acid sequence of a u-PA cleavage site
SGRSA

SEQ ID NO: 60 - protein, 6 aa, Artificial Sequence
  Amino acid sequence of a u-PA cleavage site
GSGRSA

SEQ ID NO: 61 - protein, 5 aa, Artificial Sequence
  Amino acid sequence of a u-PA cleavage site
SGKSA

SEQ ID NO: 62 - protein, 5 aa, Artificial Sequence
  Amino acid sequence of a u-PA cleavage site
SGRSS

SEQ ID NO: 63 - protein, 5 aa, Artificial Sequence
  Amino acid sequence of a u-PA cleavage site
SGRRA

SEQ ID NO: 64 - protein, 5 aa, Artificial Sequence
  Amino acid sequence of a u-PA cleavage site
SGRNA

SEQ ID NO: 65 - protein, 5 aa, Artificial Sequence
  Amino acid sequence of a u-PA cleavage site
SGRKA

SEQ ID NO: 66 - protein, 6 aa, Artificial Sequence
  Amino acid sequence of a tPA cleavage site
QRGRSA

SEQ ID NO: 67 - protein, 6 aa, Artificial Sequence
  Amino acid sequence of a cathepsin B cleavage site
TQGAAA

SEQ ID NO: 68 - protein, 6 aa, Artificial Sequence
  Amino acid sequence of a cathepsin B cleavage site
GAAAAA

SEQ ID NO: 69 - protein, 6 aa, Artificial Sequence
  Amino acid sequence of a cathepsin B cleavage site
GAGAAG

SEQ ID NO: 70 - protein, 6 aa, Artificial Sequence
  Amino acid sequence of a cathepsin B cleavage site
AAAAAG

SEQ ID NO: 71 - protein, 6 aa, Artificial Sequence
  Amino acid sequence of a cathepsin B cleavage site
LCGAAI

SEQ ID NO: 72 - protein, 6 aa, Artificial Sequence
  Amino acid sequence of a cathepsin B cleavage site
FAQALG

SEQ ID NO: 73 - protein, 6 aa, Artificial Sequence
  Amino acid sequence of a cathepsin B cleavage site
LAAANP

SEQ ID NO: 74 - protein, 6 aa, Artificial Sequence
  Amino acid sequence of a cathepsin B cleavage site
LLQANP

SEQ ID NO: 75 - protein, 6 aa, Artificial Sequence
  Amino acid sequence of a cathepsin B cleavage site
LAAANP

SEQ ID NO: 76 - protein, 6 aa, Artificial Sequence
  Amino acid sequence of a cathepsin B cleavage site
LYGAQF

SEQ ID NO: 77 - protein, 6 aa, Artificial Sequence
  Amino acid sequence of a cathepsin B cleavage site
LSQAQG

SEQ ID NO: 78 - protein, 6 aa, Artificial Sequence
  Amino acid sequence of a cathepsin B cleavage site
ASAASG

SEQ ID NO: 79 - protein, 6 aa, Artificial Sequence
  Amino acid sequence of a cathepsin B cleavage site
FLGASL

SEQ ID NO: 80 - protein, 6 aa, Artificial Sequence
  Amino acid sequence of a cathepsin B cleavage site
AYGATG

SEQ ID NO: 81 - protein, 6 aa, Artificial Sequence
  Amino acid sequence of a cathepsin B cleavage site
LAQATG

SEQ ID NO: 82 - protein, 6 aa, Artificial Sequence
  Amino acid sequence of a furin cleavage site
RRRRRR

SEQ ID NO: 83 - protein, 91 aa, Artificial Sequence
  Amino acid sequence of a fragment of human fibronectin
VPRDLEVVAATPTSLLISWDAPHHGVAYYRITYGETGGNSPVQEFTVPGSKSTATISGLKPGVDYTINVYAVLAYPRGYPLSKPISINYRT

SEQ ID NO: 84 - protein, 232 aa, Artificial Sequence
  Amino acid sequence of a human IgG1 Fc polypeptide chain
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 228 aa, Artificial Sequence
  Amino acid sequence of a human IgG2 Fc polypeptide chain
ERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 86 - protein, 279 aa, Artificial Sequence
  Amino acid sequence of a human IgG3 Fc polypeptide chain
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 87 - protein, 229 aa, Artificial Sequence
  Amino acid sequence of a human IgG4 Fc polypeptide chain
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 88 - protein, 5 aa, Artificial Sequence
  Amino acid sequence of a linker
  REPEAT (1)..(5): Repetition of amino acid sequence possible from 1 to 10 times
GGGGS

SEQ ID NO: 89 - protein, 5 aa, Artificial Sequence
  Amino acid sequence of a linker
TVAAP

SEQ ID NO: 90 - protein, 6 aa, Artificial Sequence
  Amino acid sequence of a linker
ASTKGP

SEQ ID NO: 91 - protein, 8 aa, Artificial Sequence
  Amino acid sequence of a linker
GGGGSAAA

SEQ ID NO: 92 - protein, 15 aa, Artificial Sequence
  Amino acid sequence of a linker
GGGGSGGGGSGGGGS

SEQ ID NO: 93 - protein, 580 aa, Artificial Sequence
  Amino acid sequence of a second polypeptide chain of aCD3-aHER2-Xbody
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSELVMTQTPSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLEIKASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVGGGGSAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 94 - protein, 484 aa, Artificial Sequence
  Amino acid sequence of a second polypeptide chain of aCD3-aHER2-mxb
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLAAAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 95 - protein, 6 aa, Artificial Sequence
  Amino acid sequence of a matrix metalloproteinase-11 (MMP-11) cleavage site
AANLRN

SEQ ID NO: 96 - protein, 6 aa, Artificial Sequence
  Amino acid sequence of an MMP-11 cleavage site
AQAYVK

SEQ ID NO: 97 - protein, 6 aa, Artificial Sequence
  Amino acid sequence of an MMP-11 cleavage site
AANYMR

SEQ ID NO: 98 - protein, 6 aa, Artificial Sequence
  Amino acid sequence of an MMP-11 cleavage site
AAALTR

SEQ ID NO: 99 - protein, 6 aa, Artificial Sequence
  Amino acid sequence of an MMP-11 cleavage site
AQNLMR

SEQ ID NO: 100 - protein, 6 aa, Artificial Sequence
  Amino acid sequence of an MMP-11 cleavage site
AANYTK

SEQ ID NO: 101 - protein, 12 aa, Artificial Sequence
  Amino acid insertion that extends half life of an Fc region
GGCVFNMFNCGG

SEQ ID NO: 102 - protein, 12 aa, Artificial Sequence
  Amino acid insertion that extends half life of an Fc region
GGCHLPFAVCGG

SEQ ID NO: 103 - protein, 12 aa, Artificial Sequence
  Amino acid insertion that extends half life of an Fc region
GGCGHEYMWCGG

SEQ ID NO: 104 - protein, 12 aa, Artificial Sequence
  Amino acid insertion that extends half life of an Fc region
GGCWPLQDYCGG

SEQ ID NO: 105 - protein, 12 aa, Artificial Sequence
  Amino acid insertion that extends half life of an Fc region
GGCMQMNKWCGG

SEQ ID NO: 106 - protein, 12 aa, Artificial Sequence
  Amino acid insertion that extends half life of an Fc region
GGCDGRTKYCGG

SEQ ID NO: 107 - protein, 12 aa, Artificial Sequence
  Amino acid insertion that extends half life of an Fc region
GGCALYPTNCGG

SEQ ID NO: 108 - protein, 12 aa, Artificial Sequence
  Amino acid insertion that extends half life of an Fc region
GGCGKHWHQCGG

SEQ ID NO: 109 - protein, 12 aa, Artificial Sequence
  Amino acid insertion that extends half life of an Fc region
GGCHSFKHFCGG

SEQ ID NO: 110 - protein, 12 aa, Artificial Sequence
  Amino acid insertion that extends half life of an Fc region
GGCQGMWTWCGG

SEQ ID NO: 111 - protein, 14 aa, Artificial Sequence
  Amino acid insertion that extends half life of an Fc region
GGCAQQWHHEYCGG

SEQ ID NO: 112 - protein, 12 aa, Artificial Sequence
  Amino acid insertion that extends half life of an Fc region
GGCERFHHACGG

SEQ ID NO: 113 - protein, 9 aa, Artificial Sequence
  Synthetic Polypeptide
  misc_feature (2)..(9): Xaa can be any naturally occurring amino acid
CXXXXXXXX

SEQ ID NO: 114 - protein, 5 aa, Artificial Sequence
  Synthetic Polypeptide
LEWIG

SEQ ID NO: 115 - protein, 4 aa, Artificial Sequence
  Synthetic Polypeptidde
  misc_feature (3)..(3): Xaa can be any naturally occurring amino acid
WGXG

SEQ ID NO: 116 - protein, 4 aa, Artificial Sequence
  Synthetic Polypeptide
  misc_feature (3)..(3): Xaa can be any naturally occurring amino acid
FGXG

SEQ ID NO: 117 - protein, 8 aa, Artificial Sequence
  Synthetic Polypeptide
  MISC_FEATURE (1)..(1): Xaa is Ala, Lys, or Asp
  MISC_FEATURE (2)..(2): Xaa is Ala, Ser, or Gly
  MISC_FEATURE (3)..(3): Xaa is Pro, Ala, or Val
  MISC_FEATURE (4)..(4): Xaa is Gly or Pro
  MISC_FEATURE (5)..(5): Xaa is Leu, Ile, Phe, or Tyr
  MISC_FEATURE (6)..(6): Xaa is Lys, Ala, Val, Ile, or Tyr
  MISC_FEATURE (7)..(7): Xaa is Ala, Ser, or Gly
  MISC_FEATURE (8)..(8): Xaa is Ala, Lys, or Asp
XXXXXXXX

SEQ ID NO: 118 - protein, 6 aa, Artificial Sequence
  Synthetic Polypeptide
  MISC_FEATURE (1)..(1): Xaa is Gly, Phe, Leu, or Pro
  MISC_FEATURE (2)..(2): Xaa is Ala, Val, Tyr, Phe, or Ile
  MISC_FEATURE (3)..(3): Xaa is Gly, Ala, Met, Gln, or Tyr
  MISC_FEATURE (4)..(4): Xaa is Phe, Gly, Ile, Val, or Lys
  MISC_FEATURE (5)..(5): Xaa is Val, Ile, Gly, Thr, or Ala
  MISC_FEATURE (6)..(6): Xaa is Gly
XXXXXX